SEQUENCE LISTING (361 sequences)

SEQ ID NO: 1 - DNA, 30 bp, synthetic construct
  source 1..30: mol_type = other DNA
gtaattgcggcaagaagaattgtttctgtc

SEQ ID NO: 2 - DNA, 30 bp, synthetic construct
  source 1..30: mol_type = other DNA
gtaattgcggcaggaggaattgtttctgtc

SEQ ID NO: 3 - DNA, 30 bp, synthetic construct
  source 1..30: mol_type = other DNA
ggcaagagacaccaaagacactaccagcat

SEQ ID NO: 4 - DNA, 20 bp, synthetic construct
  source 1..20: mol_type = other DNA
ccaggctgactcaaccgctg

SEQ ID NO: 5 - DNA, 20 bp, synthetic construct
  source 1..20: mol_type = other DNA
acccatgttgcctgggcacc

SEQ ID NO: 6 - DNA, 20 bp, synthetic construct
  source 1..20: mol_type = other DNA
gtaggcttgaaccttgtcaa

SEQ ID NO: 7 - DNA, 20 bp, synthetic construct
  source 1..20: mol_type = other DNA
tctccccaggctgactcaac

SEQ ID NO: 8 - DNA, 20 bp, synthetic construct
  source 1..20: mol_type = other DNA
cagaagcgcacatgagagat

SEQ ID NO: 9 - DNA, 20 bp, synthetic construct
  source 1..20: mol_type = other DNA
gaagcgcacatgagagattg

SEQ ID NO: 10 - DNA, 20 bp, synthetic construct
  source 1..20: mol_type = other DNA
agtgtgggtacagacacaaa

SEQ ID NO: 11 - DNA, 20 bp, synthetic construct
  source 1..20: mol_type = other DNA
cagacacaaatatgatctgc

SEQ ID NO: 12 - DNA, 20 bp, synthetic construct
  source 1..20: mol_type = other DNA
atatgatctgcaggacccag

SEQ ID NO: 13 - DNA, 20 bp, synthetic construct
  source 1..20: mol_type = other DNA
gtaattgcggcaagaagaat

SEQ ID NO: 14 - DNA, 20 bp, synthetic construct
  source 1..20: mol_type = other DNA
aggctgtgcatgggagttag

SEQ ID NO: 15 - DNA, 20 bp, synthetic construct
  source 1..20: mol_type = other DNA
cgctggtccacaatggctag

SEQ ID NO: 16 - DNA, 20 bp, synthetic construct
  source 1..20: mol_type = other DNA
gctggctctgcttgaaggtc

SEQ ID NO: 17 - DNA, 3085 bp, Homo sapiens
  source 1..3085: mol_type = genomic DNA
acaaaaaagcttttacgaggtatcagcacttttctttcattagggggaaggcgtgaggaaagtaccaaacagcagcggagttttaaactttaaatagacaggtctgagtgcctgaacttgccttttcattttacttcatcctccaaggagttcaatcacttggcgtgacttcactacttttaagcaaaagagtggtgcccaggcaacatgggtgactggagcgccttaggcaaactccttgacaaggttcaagcctactcaactgctggagggaaggtgtggctgtcagtacttttcattttccgaatcctgctgctggggacagcggttgagtcagcctggggagatgagcagtctgcctttcgttgtaacactcagcaacctggttgtgaaaatgtctgctatgacaagtctttcccaatctctcatgtgcgcttctgggtcctgcagatcatatttgtgtctgtacccacactcttgtacctggctcatgtgttctatgtgatgcgaaaggaagagaaactgaacaagaaagaggaagaactcaaggttgcccaaactgatggtgtcaatgtggacatgcacttgaagcagattgagataaagaagttcaagtacggtattgaagagcatggtaaggtgaaaatgcgaggggggttgctgcgaacctacatcatcagtatcctcttcaagtctatctttgaggtggccttcttgctgatccagtggtacatctatggattcagcttgagtgctgtttacacttgcaaaagagatccctgcccacatcaggtggactgtttcctctctcgccccacggagaaaaccatcttcatcatcttcatgctggtggtgtccttggtgtccctggccttgaatatcattgaactcttctatgttttcttcaagggcgttaaggatcgggttaagggaaagagcgacccttaccatgcgaccagtggtgcgctgagccctgccaaagactgtgggtctcaaaaatatgcttatttcaatggctgctcctcaccaaccgctcccctctcgcctatgtctcctcctgggtacaagctggttactggcgacagaaacaattcttcttgccgcaattacaacaagcaagcaagtgagcaaaactgggctaattacagtgcagaacaaaatcgaatggggcaggcgggaagcaccatctctaactcccatgcacagccttttgatttccccgatgataaccagaattctaaaaaactagctgctggacatgaattacagccactagccattgtggaccagcgaccttcaagcagagccagcagtcgtgccagcagcagacctcggcctgatgacctggagatctagatacaggcttgaaagcatcaagattccactcaattgtggagaagaaaaaaggtgctgtagaaagtgcaccaggtgttaattttgatccggtggaggtggtactcaacagccttattcatgaggcttagaaaacacaaagacattagaatacctaggttcactgggggtgtatggggtagatgggtggagagggaggggataagagaggtgcatgttggtatttaaagtagtggattcaaagaacttagattataaataagagttccattaggtgatacatagataagggctttttctccccgcaaacacccctaagaatggttctgtgtatgtgaatgagcgggtggtaattgtggctaaatatttttgttttaccaagaaactgaaataattctggccaggaataaatacttcctgaacatcttaggtcttttcaacaagaaaaagacagaggattgtccttaagtccctgctaaaacattccattgttaaaatttgcactttgaaggtaagctttctaggcctgaccctccaggtgtcaatggacttgtgctactatatttttttattcttggtatcagtttaaaattcagacaaggcccacagaataagattttccatgcatttgcaaatacgtatattctttttccatccacttgcacaatatcattaccatcactttttcatcattcctcagctactactcacattcatttaatggtttctgtaaacatttttaagacagttgggatgtcacttaacattttttttttttgagctaaagtcagggaatcaagccatgcttaatatttaacaatcacttatatgtgtgtcgaagagtttgttttgtttgtcatgtattggtacaagcagatacagtataaactcacaaacacagatttgaaaataatgcacatatggtgttcaaatttgaacctttctcatggatttttgtggtgtgggccaatatggtgtttacattatataattcctgctgtggcaagtaaagcacactttttttttctcctaaaatgtttttccctgtgtatcctattatggatactggttttgttaattatgattctttattttctctcctttttttaggatatagcagtaatgctattactgaaatgaatttcctttttctgaaatgtaatcattgatgcttgaatgatagaattttagtactgtaaacaggctttagtcattaatgtgagagacttagaaaaaatgcttagagtggactattaaatgtgcctaaatgaattttgcagtaactggtattcttgggttttcctacttaatacacagtaattcagaacttgtattctattatgagtttagcagtcttttggagtgaccagcaactttgatgtttgcactaagattttatttggaatgcaagagaggttgaaagaggattcagtagtacacatacaactaatttatttgaactatatgttgaagacatctaccagtttctccaaatgccttttttaaaactcatcacagaagattggtgaaaatgctgagtatgacacttttcttcttgcatgcatgtcagctacataaacagttttgtacaatgaaaattactaatttgtttgacattccatgttaaactacggtcatgttcagcttcattgcatgtaatgtagacctagtccatcagatcatgtgttctggagagtgttctttatttaaagttttaatttagtataaacat

SEQ ID NO: 18-99 - skipped (82 entries)

SEQ ID NO: 100 - protein, 382 aa, unidentified
MGDWSALGKLLDKVQAYSTAGGKVWLSVLFIFRILLLGTAVESAWGDEQSAFRCNTQQPGCENVCYDKSFPISHVRFWVLQIIFVSVPTLLYLAHVFYVMRKEEKLNKKEEELKVAQTDGVNVDMHLKQIEIKKFKYGIEEHGKVKMRGGLLRTYIISILFKSIFEVAFLLIQWYIYGFSLSAVYTCKRDPCPHQVDCFLSRPTEKTIFIIFMLVVSLVSLALNIIELFYVFFKGVKDRVKGKSDPYHATSGALSPAKDCGSQKYAYFNGCSSPTAPLSPMSPPGYKLVTGDRNNSSCRNYNKQASEQNWANYSAEQNRMGQAGSTISNSHAQPFDFPDDNQNSKKLAAGHELQPLAIVDQRPSSRASSRASSRPRPDDLEI

SEQ ID NO: 101 - protein, 7 aa, synthetic construct
SRPTEKT

SEQ ID NO: 102 - protein, 13 aa, synthetic construct
VCYDKSFPISHVR

SEQ ID NO: 103 - protein, 12 aa, synthetic construct
INCTLQPGCNSV

SEQ ID NO: 104 - protein, 11 aa, synthetic construct
SRPTEKTIFII

SEQ ID NO: 105 - protein, 26 aa, synthetic construct
GGERAPLAADQGSVKKSSSSSKGTKK

SEQ ID NO: 106 - protein, 10 aa, synthetic construct
SRARSDDLTV

SEQ ID NO: 107 - protein, 12 aa, synthetic construct
VDCFLSRPTEKT

SEQ ID NO: 108 - protein, 9 aa, synthetic construct
KQIEIKKFK

SEQ ID NO: 109 - protein, 42 aa, synthetic construct
LLIQWYIYGFSLSAVYTCKRDPCPHQVDCFLSRPTEKTIFII

SEQ ID NO: 110 - protein, 9 aa, synthetic construct
SRPTEKTIF

SEQ ID NO: 111 - protein, 17 aa, synthetic construct
  SITE 1: note = D-amino acid
  SITE 2: note = D-amino acid
  SITE 3: note = D-amino acid
  SITE 4: note = D-amino acid
  SITE 5: note = D-amino acid
  SITE 6: note = D-amino acid
LCLRPVGGKQIEIKKFK

SEQ ID NO: 112 - protein, 12 aa, synthetic construct
ADCFLSRPTEKT

SEQ ID NO: 113 - protein, 12 aa, synthetic construct
VACFLSRPTEKT

SEQ ID NO: 114 - protein, 12 aa, synthetic construct
VDCFLSRPTAKT

SEQ ID NO: 115 - protein, 12 aa, synthetic construct
VDCFLSRPTEAT

SEQ ID NO: 116 - protein, 10 aa, synthetic construct
CFLSRPTEKT

SEQ ID NO: 117 - protein, 8 aa, synthetic construct
LSRPTEKT

SEQ ID NO: 118 - protein, 11 aa, synthetic construct
DCFISRPTEKT

SEQ ID NO: 119 - protein, 26 aa, unidentified
DGVNVEMHLKQIEIKKFKYGIEEHGK

SEQ ID NO: 120 - protein, 26 aa, synthetic construct
DGVNVEMHLKQIEIKKFKYGIEEQGK

SEQ ID NO: 121 - protein, 20 aa, unidentified
YGRKKRRQRRRKQIEIKKFK

SEQ ID NO: 122 - protein, 17 aa, unidentified
CSSPTAPLSPMSPPGYK

SEQ ID NO: 123 - protein, 11 aa, unidentified
PTAPLSPMSPP

SEQ ID NO: 124 - protein, 7 aa, synthetic construct
RPRDDEI

SEQ ID NO: 125 - protein, 9 aa, synthetic construct
SRPRDDLEI

SEQ ID NO: 126 - protein, 19 aa, synthetic construct
YGRKKRRQRRRSRPRDDEI

SEQ ID NO: 127 - protein, 18 aa, synthetic construct
YGRKKRRQRRRRPRDDEI

SEQ ID NO: 128 - protein, 6 aa, synthetic construct
  SITE 1: note = Aminated residue
  SITE 4: note = 4-hydroxyproline
  SITE 6: note = Amidated residue
GAGPPY

SEQ ID NO: 129 - protein, 6 aa, synthetic construct
  SITE 1: note = Acetylated D-amino acid
  SITE 3: note = D-4-hydroxyproline
  SITE 5: note = D-amino acid
  SITE 6: note = Aminated residue
YPPGAG

SEQ ID NO: 130 - protein, 35 aa, unidentified
ESAWGDEQSAFRCNTQQPGCENVCYDKSFPISHVR

SEQ ID NO: 131 - protein, 36 aa, unidentified
GESIYYDEQSKFVCNTEQPGCENVCYDAFAPLSHVR

SEQ ID NO: 132 - protein, 38 aa, unidentified
LLIQWYIYGFSLSAVYTCKRDPCPHQVDCFLSRPTEKT

SEQ ID NO: 133 - protein, 38 aa, unidentified
LIGQYFLYGFQVHPFYVCSRLPCHPKIDCFISRPTEKT

SEQ ID NO: 134 - protein, 15 aa, synthetic construct
VFFKGVKDRVKGRSD

SEQ ID NO: 135 - protein, 8 aa, synthetic construct
RPRDDLEI

SEQ ID NO: 136 - protein, 9 aa, synthetic construct
SRPRDDLEI

SEQ ID NO: 137 - protein, 25 aa, synthetic construct
RQPKIWFPNRRKPWKKRPRPDDLEI

SEQ ID NO: 138 - protein, 13 aa, synthetic construct
GHGDPLHLEEVKC

SEQ ID NO: 139 - protein, 24 aa, synthetic construct
YGRKKRRQRRRGHGDPLHLEEVKC

SEQ ID NO: 140 - protein, 20 aa, synthetic construct
PSSRASSRASSRPRPDDLEI

SEQ ID NO: 141 - protein, 9 aa, synthetic construct
RPRPDDLEI

SEQ ID NO: 142 - protein, 9 aa, synthetic construct
RPRPDDLEV

SEQ ID NO: 143 - protein, 9 aa, synthetic construct
RPRPDDVPV

SEQ ID NO: 144 - protein, 9 aa, synthetic construct
KARSDDLSV

SEQ ID NO: 145 - protein, 40 aa, synthetic construct
QKPEVPNGVSPGHRLPHGYHSDKRRLSKASSKARSDDLSV

SEQ ID NO: 146 - protein, 36 aa, synthetic construct
RQPKIWFPNRRKPWKKPSSRASSRASSRPRPDDLEI

SEQ ID NO: 147 - protein, 36 aa, synthetic construct; identical to SEQ ID NO: 146

SEQ ID NO: 148 - protein, 25 aa, synthetic construct
RQPKIWFPNRRKPWKKRPRPDDLEI

SEQ ID NO: 149 - protein, 25 aa, synthetic construct
RQPKIWFPNRRKPWKKRPRPDDLEV

SEQ ID NO: 150 - protein, 25 aa, synthetic construct
RQPKIWFPNRRKPWKKRPRPDDVPV

SEQ ID NO: 151 - protein, 25 aa, synthetic construct
RQPKIWFPNRRKPWKKKARSDDLSV

SEQ ID NO: 152 - protein, 9 aa, synthetic construct
RPKPDDLDI

SEQ ID NO: 153 - protein, 20 aa, synthetic construct
GRKKRRQRPPQRPRPDDLEI

SEQ ID NO: 154 - protein, 25 aa, synthetic construct
RQIKIWFQNRRMKWKKRPRPDDLEI

SEQ ID NO: 155 - protein, 25 aa, synthetic construct
RQIAIWFQNRRMKWAARPRPDDLEI

SEQ ID NO: 156 - protein, 18 aa, synthetic construct
RKKRRQRRRRPRPDDLEI

SEQ ID NO: 157 - protein, 30 aa, synthetic construct
TRSSRAGLQFPVGRVHRLLRKRPRPDDLEI

SEQ ID NO: 158 - protein, 35 aa, synthetic construct
GWTLNSAGYLLGKINKALAALAKKILRPRPDDLEI

SEQ ID NO: 159 - skipped

SEQ ID NO: 160 - protein, 25 aa, synthetic construct
AAVALLPAVLLALLAPRPRPDDLEI

SEQ ID NO: 161 - protein, 19 aa, synthetic construct
VPMLKPMLKERPRPDDLEI

SEQ ID NO: 162 - protein, 37 aa, synthetic construct
MANLGYWLLALFVTMWTDVGLCKKRPKPRPRPDDLEI

SEQ ID NO: 163 - protein, 27 aa, synthetic construct
LLIILRRRIRKQAHAHSKRPRPDDLEI

SEQ ID NO: 164 - protein, 30 aa, synthetic construct
KETWWETWWTEWSQPKKKRKVRPRPDDLEI

SEQ ID NO: 165 - protein, 27 aa, synthetic construct
RGGRLSYSRRRFSTSTGRRPRPDDLEI

SEQ ID NO: 166 - protein, 24 aa, synthetic construct
SDLWEMMMVSLACQYRPRPDDLEI

SEQ ID NO: 167 - protein, 21 aa, synthetic construct
TSPLNIHNGQKLRPRPDDLEI

SEQ ID NO: 168 - protein, 12 aa, synthetic construct
VDCFLSRPTEKT

SEQ ID NO: 169 - protein, 27 aa, synthetic construct
KLALKLALKALKAALKLARPRPDDLEI

SEQ ID NO: 170-173 - skipped (4 entries)

SEQ ID NO: 174 - protein, 10 aa, synthetic construct
VDCFLSRPTE

SEQ ID NO: 175 - protein, 8 aa, synthetic construct
VDCFLSRP

SEQ ID NO: 176 - protein, 6 aa, synthetic construct
VDCFLS

SEQ ID NO: 177 - protein, 23 aa, synthetic construct
GRKKRRQRPPQVDCFLSRPTEKT

SEQ ID NO: 178 - protein, 28 aa, synthetic construct
RQIKIWFQNRRMKWKKVDCFLSRPTEKT

SEQ ID NO: 179 - protein, 28 aa, synthetic construct
RQIAIWFQNRRMKWAAVDCFLSRPTEKT

SEQ ID NO: 180 - protein, 21 aa, synthetic construct
RKKRRQRRRVDCFLSRPTEKT

SEQ ID NO: 181 - protein, 33 aa, synthetic construct
TRSSRAGLQFPVGRVHRLLRKVDCFLSRPTEKT

SEQ ID NO: 182 - protein, 38 aa, synthetic construct
GWTLNSAGYLLGKINKALAALAKKILVDCFLSRPTEKT

SEQ ID NO: 183 - protein, 30 aa, synthetic construct
KLALKLALKALKAALKLAVDCFLSRPTEKT

SEQ ID NO: 184 - protein, 28 aa, synthetic construct
AAVALLPAVLLALLAPVDCFLSRPTEKT

SEQ ID NO: 185 - protein, 22 aa, synthetic construct
VPMLKPMLKEVDCFLSRPTEKT

SEQ ID NO: 186 - protein, 40 aa, synthetic construct
MANLGYWLLALFVTMWTDVGLCKKRPKPVDCFLSRPTEKT

SEQ ID NO: 187 - protein, 30 aa, synthetic construct
LLIILRRRIRKQAHAHSKVDCFLSRPTEKT

SEQ ID NO: 188 - protein, 33 aa, synthetic construct
KETWWETWWTEWSQPKKKRKVVDCFLSRPTEKT

SEQ ID NO: 189 - protein, 30 aa, synthetic construct
RGGRLSYSRRRFSTSTGRVDCFLSRPTEKT

SEQ ID NO: 190 - protein, 27 aa, synthetic construct
SDLWEMMMVSLACQYVDCFLSRPTEKT

SEQ ID NO: 191 - protein, 24 aa, synthetic construct
TSPLNIHNGQKLVDCFLSRPTEKT

SEQ ID NO: 192 - protein, 11 aa, synthetic construct
FEVAFLLIQWI

SEQ ID NO: 193 - protein, 11 aa, synthetic construct
LLIQWYIGFSL

SEQ ID NO: 194 - protein, 16 aa, synthetic construct
SLSAVYTCKRDPCPHQ

SEQ ID NO: 195 - skipped

SEQ ID NO: 196 - protein, 13 aa, synthetic construct
LGTAVESAWGDEQ

SEQ ID NO: 197 - protein, 12 aa, synthetic construct
QSAFRCNTQQPG

SEQ ID NO: 198 - protein, 12 aa, synthetic construct
QQPGCENVCYDK

SEQ ID NO: 199 - skipped

SEQ ID NO: 200 - protein, 18 aa, synthetic construct
KRDPCHQVDCFLSRPTEK

SEQ ID NO: 201-236 - skipped (36 entries)

SEQ ID NO: 237 - protein, 46 aa, synthetic construct
LTAVGGESIYYDEQSKFVCNTEQPGCENVCYDAFAPLSHVRFWVFQ

SEQ ID NO: 238 - protein, 15 aa, synthetic construct
LTAVGGESIYYDEQS

SEQ ID NO: 239 - protein, 13 aa, synthetic construct
DEQSKFVCNTEQP

SEQ ID NO: 240 - skipped

SEQ ID NO: 241 - protein, 13 aa, synthetic construct
TEQPGCENVCYDA

SEQ ID NO: 242 - protein, 13 aa, synthetic construct
VCYDAFAPLSHVR

SEQ ID NO: 243 - protein, 12 aa, synthetic construct
APLSHVRFWVFQ

SEQ ID NO: 244 - skipped

SEQ ID NO: 245 - protein, 11 aa, synthetic construct
FEVGFLIGQYF

SEQ ID NO: 246 - protein, 12 aa, synthetic construct
LIGQYFLYGFQV

SEQ ID NO: 247 - protein, 14 aa, synthetic construct
GFQVHPFYVCSRLP

SEQ ID NO: 248 - protein, 12 aa, synthetic construct
SRLPCHPKIDCF

SEQ ID NO: 249 - protein, 12 aa, synthetic construct
IDCFISRPTEKT

SEQ ID NO: 250 - protein, 11 aa, synthetic construct
SRPTEKTIFLL

SEQ ID NO: 251 - skipped

SEQ ID NO: 252 - protein, 10 aa, synthetic construct
YVCSRLPCHP

SEQ ID NO: 253 - protein, 11 aa, synthetic construct
QVHPFYVCSRL

SEQ ID NO: 254 - protein, 13 aa, synthetic construct
FEVGFLIGQYFLY

SEQ ID NO: 255 - protein, 12 aa, synthetic construct
GQYFLYGFQVHP

SEQ ID NO: 256 - protein, 12 aa, synthetic construct
GFQVHPFYVCSR

SEQ ID NO: 257 - protein, 12 aa, synthetic construct
AVGGESIYYDEQ

SEQ ID NO: 258 - protein, 12 aa, synthetic construct
YDEQSKFVCNTE

SEQ ID NO: 259 - protein, 12 aa, synthetic construct
NTEQPGCENVCY

SEQ ID NO: 260 - protein, 12 aa, synthetic construct
CYDAFAPLSHVR

SEQ ID NO: 261 - protein, 12 aa, synthetic construct
FAPLSHVRFWVF

SEQ ID NO: 262 - protein, 5 aa, synthetic construct
LIGQY

SEQ ID NO: 263 - protein, 5 aa, synthetic construct
QVHPF

SEQ ID NO: 264 - protein, 5 aa, synthetic construct
YVCSR

SEQ ID NO: 265 - protein, 5 aa, synthetic construct
SRLPC

SEQ ID NO: 266 - protein, 5 aa, synthetic construct
LPCHP

SEQ ID NO: 267 - protein, 5 aa, synthetic construct
GESIY

SEQ ID NO: 268 - protein, 6 aa, synthetic construct
YDEQSK

SEQ ID NO: 269 - protein, 6 aa, synthetic construct
SKFVCN

SEQ ID NO: 270 - protein, 8 aa, synthetic construct
TEQPGCEN

SEQ ID NO: 271 - protein, 8 aa, synthetic construct
VCYDAFAP

SEQ ID NO: 272 - protein, 10 aa, synthetic construct
LSHVRFWVFQ

SEQ ID NO: 273 - protein, 14 aa, synthetic construct
LIQYFLYGFQVHPF

SEQ ID NO: 274 - protein, 13 aa, synthetic construct
VHPFYCSRLPCHP

SEQ ID NO: 275 - protein, 22 aa, synthetic construct
VGGESIYYDEQSKFVCNTEQPG

SEQ ID NO: 276 - protein, 22 aa, synthetic construct
TEQPGCENVCYDAFAPLSHVRF

SEQ ID NO: 277 - protein, 14 aa, synthetic construct
AFAPLSHVRFWVFQ

SEQ ID NO: 278 - protein, 16 aa, synthetic construct
IDCFISRPTEKTIFLL

SEQ ID NO: 279 - protein, 11 aa, synthetic construct
DCFISRPTEKT

SEQ ID NO: 280-290 - skipped (11 entries)

SEQ ID NO: 291 - protein, 12 aa, synthetic construct
ADCFLSRPTEKT

SEQ ID NO: 292 - protein, 12 aa, synthetic construct
VACFLSRPTEKT

SEQ ID NO: 293 - protein, 12 aa, synthetic construct
VDCFLSRPTAKT

SEQ ID NO: 294 - protein, 12 aa, synthetic construct
VDCFLSRPTEAT

SEQ ID NO: 295-299 - skipped (5 entries)

SEQ ID NO: 300 - protein, 26 aa, synthetic construct
DGVNVEMHLKQIEIKKFKYGIEEHGK

SEQ ID NO: 301 - protein, 26 aa, synthetic construct
DGVNVEMHLKQIEIKKFKYGIEEQGK

SEQ ID NO: 302 - protein, 20 aa, synthetic construct
YGRKKRRQRRRKQIEIKKFK

SEQ ID NO: 303 - protein, 17 aa, synthetic construct
CSSPTAPLSPMSPPGYK

SEQ ID NO: 304 - protein, 11 aa, synthetic construct
PTAPLSPMSPP

SEQ ID NO: 305-308 - skipped (4 entries)

SEQ ID NO: 309 - protein, 6 aa, synthetic construct
  SITE 1: note = Aminated residue
  SITE 4: note = 4-hydroxyproline
  SITE 6: note = Amidated residue
GAGPPY

SEQ ID NO: 310 - protein, 6 aa, synthetic construct
  SITE 1: note = Acetylated D-amino acid
  SITE 3: note = D-4-hydroxyproline
  SITE 5: note = D-amino acid
  SITE 6: note = Aminated residue
YPPGAG

SEQ ID NO: 311 - protein, 28 aa, synthetic construct
RVIRVWFQNKRCKDKKVDCFLSRPTEKT

SEQ ID NO: 312 - protein, 39 aa, synthetic construct
GALFLGFLGAAGSTMGAWSQPKKKRKVVDCFLSRPTEKT

SEQ ID NO: 313 - protein, 39 aa, synthetic construct
GALFLAFLAAALSLMGLWSQPKKKRRVVDCFLSRPTEKT

SEQ ID NO: 314-342 - skipped (29 entries)

SEQ ID NO: 343 - protein, 39 aa, synthetic construct
MYVFYVMYDGFSMQRLVKCNAWPCPNTVDCFVSRPTEKT

SEQ ID NO: 344 - protein, 39 aa, synthetic construct
MYVFYFLYNGYHLPWVLKCGIDPCPNLVDCFISRPTEKT

SEQ ID NO: 345 - protein, 39 aa, synthetic construct
LYIFHRLYKDYDMPRVVACSVEPCPHTVDCYISRPTEKK

SEQ ID NO: 346 - protein, 40 aa, synthetic construct
LYLLHTLWHGFNMPRLVQCANVAPCPNIVDCYIARPTEKK

SEQ ID NO: 347 - protein, 39 aa, synthetic construct
LYVFHSFYPKYILPPVVKCHADPCPNIVDCFISKPSEKN

SEQ ID NO: 348 - protein, 39 aa, synthetic construct
MYVFYLLYPGYAMVRLVKCDVYPCPNTVDCFVSRPTEKT

SEQ ID NO: 349 - protein, 32 aa, synthetic construct
LYGWTMEPVFVCQRAPCPYLVDCFVSRPTEKT

SEQ ID NO: 350 - protein, 32 aa, synthetic construct; identical to SEQ ID NO: 349

SEQ ID NO: 351 - protein, 38 aa, synthetic construct
GALHYFLFGFLAPKKFPCTRPPCTGVVDCYVSRPTEKS

SEQ ID NO: 352 - skipped

SEQ ID NO: 353 - protein, 38 aa, synthetic construct
IAGQYFLYGFELKPLYRCDRWPCPNTVDCFISRPTEKT

SEQ ID NO: 354 - protein, 38 aa, synthetic construct
LVGQYLLYGFEVRPFFPCSRQPCPHVVDCFVSRPTEKT

SEQ ID NO: 355 - protein, 38 aa, synthetic construct
IVGQYFIYGIFLTTLHVCRRSPCPHPVNCYVSRPTEKN

SEQ ID NO: 356 - DNA, 30 bp, synthetic construct; identical to SEQ ID NO: 1
  source 1..30: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  modified_base 4: mod_base = OTHER; note = Nucleotide with phosphorothioate linkage
  modified_base 26: mod_base = OTHER; note = Nucleotide with phosphorothioate linkage
  modified_base 27: mod_base = OTHER; note = Nucleotide with phosphorothioate linkage
  modified_base 28: mod_base = OTHER; note = Nucleotide with phosphorothioate linkage
  modified_base 29: mod_base = OTHER; note = Nucleotide with phosphorothioate linkage

SEQ ID NO: 357 - DNA, 30 bp, synthetic construct; identical to SEQ ID NO: 1
  source 1..30: mol_type = other DNA
  misc_difference 1: note = Nucleotide with any modified sugar moiety
  misc_difference 2: note = Nucleotide with any modified sugar moiety
  misc_difference 3: note = Nucleotide with any modified sugar moiety
  misc_difference 4: note = Nucleotide with any modified sugar moiety
  misc_difference 27: note = Nucleotide with any modified sugar moiety
  misc_difference 28: note = Nucleotide with any modified sugar moiety
  misc_difference 29: note = Nucleotide with any modified sugar moiety
  misc_difference 30: note = Nucleotide with any modified sugar moiety

SEQ ID NO: 358 - protein, 14 aa, synthetic construct
  VARIANT 1: note = Any amino acid, wherein the side chain can be hydrogen or n-dodecyl
  VARIANT 2: note = Any amino acid, wherein the side chain can be hydrogen or n-dodecyl
XXLDCFLSRPTEKT

SEQ ID NO: 359 - protein, 4 aa, synthetic construct
SHVR

SEQ ID NO: 360 - protein, 6 aa, synthetic construct
SRPTEK

SEQ ID NO: 361 - protein, 4 aa, synthetic construct
KKFK

(19) United States
(12) Patent Application Publication
(10) Pub. No.: US 2026/0272972 A1
(43) Pub. Date: Sep. 17, 2026

(54) TREATMENTS FOR OCULAR SURFACE DISORDERS

(71) Applicant: GLAUKOS CORPORATION, Aliso Viejo, CA (US)

(72) Inventors: Shawn A. SCRANTON, Carlsbad, CA (US); Colin Richard GREEN, Auckland (NZ); Brian LEVY, Tampa, FL (US)

(21) Appl. No.: 19/686,276

(22) Filed: May 22, 2026

Related U.S. Application Data
(63) Continuation of application No. 18/224,017, filed on Jul. 19, 2023, now Pat. No. 12,667,586.
(60) Provisional application No. 63/450,597, filed on Mar. 7, 2023.
(60) Provisional application No. 63/521,298, filed on Jun. 15, 2023.

(51) Int. Cl. A61K 31/7125; A61K 9/00; A61K 47/10; A61P 27/02; C12N 15/11; C12N 15/113

(52) U.S. Cl. CPC A61K 31/7125; A61K 9/0051; A61K 47/10; A61P 27/02; C12N 15/111; C12N 15/113

(57) ABSTRACT

The inventions relate to compounds and compositions comprising connexin modulators and their use in dose regimens for the treatment of non-healing or persistent ocular surface or corneal defects and disorders.

### RELATED APPLICATIONS

This application is a continuation application of U.S. patent application Ser. No. 18/224,017, filed Jul. 19, 2023, which claims priority to U.S. Provisional Application No. 63/521,298, filed Jun. 15, 2023, and U.S. Provisional Application No. 63/450,597, filed Mar. 7, 2023, the contents of each of which is herein incorporated by reference in its entirety.

### SEQUENCE LISTING

The instant application contains a Sequence Listing which has been submitted electronically in XML format and is hereby incorporated by reference in its entirety. Said XML copy, created on May 22, 2026, is named 69307-702_301_SL.xml and is 215,987 bytes in size.

### FIELD

The inventions relate to connexin modulators and modulation and ocular surface defects or disorders.

### INCORPORATION BY REFERENCE

All publications, patents, related applications, and other written or electronic materials mentioned, identified or referred to herein, including each and every United States patent, United States patent application publication, non-U.S. patent, non-U.S. and PCT published application, article and other document cited or noted herein, and all those listed as References Cited in any patent or patents that issue herefrom, are hereby incorporated by reference in their entirety. The information incorporated is as much a part of this application, and all patents issuing therefrom or claiming priority thereto, as if all of the text and other content was repeated in the application or patent, will be treated as part of the text and content of this application as filed and any patent issuing therefrom or claiming priority thereto, and any portion of any material incorporated by reference may be included herein by amendment if required. In the event of inconsistent usages between this document and any document incorporated by reference, the usage in the incorporated reference(s) should be considered supplementary to that of this document; for irreconcilable inconsistencies, including definitions appearing in patents or patent applications, the usage in this document controls.

### TECHNICAL BACKGROUND

The following includes information that may be useful in understanding the present inventions. It is not an admission that any of the information, publications or documents specifically or implicitly referenced herein is prior art, or important, to the inventions described and claimed herein.

Gap junctions are specialized intercellular connections found between most animal cell-types. They are expressed in virtually all tissues of the body, except for mature skeletal muscle and mobile cell types such as sperm and erythrocytes and provide regulated physical communication between cells by directly linking the interiors of adjoining cells, allowing various molecules, ions and electrical impulses to directly pass through.

One gap junction channel is composed of two connexin hemichannels (connexons), which connect across the intercellular space between adjacent cells. Each hemichannel of a gap junction resides in the adjacent cell membrane, and each hemichannel is formed by the covalent oligomerization of six individual connexin (Cx) proteins. See, e.g., Yeager (1998) Structure of cardiac gap junction intercellular channels, J Struct Biol 121:231-245. Hemichannels can comprise one or more different connexin proteins but are usually in the form of homohexamers.

The human connexin family of genes and proteins now numbers 21. They usually weigh between 25 and 60 kDa and have an average length of 380 amino acids. All connexins share a common structure as a 4-pass transmembrane (TM) protein that includes several domains, namely, a short intracellular N-terminus (NT), an intracellular loop (IL) and a C-terminus (CT) that is also localized in the cytoplasm, plus two extracellular loops (EL1 and EL2) located outside the cell. The cytoplasmic carboxy terminus can vary considerably in length. See, e.g., Unger, et al. (1999) Electron cryo-crystallography of a recombinant cardiac gap junction channel, Novartis Found Symp 219:22-30 & discussion 31-43; Leith, E, et al., The connexin 43 C-terminus: A tail of many tales. Biochimica et Biophysica Acta Vol. (1): 48-64 (January 2018).

Connexin proteins are commonly named according to their molecular weights, e.g. Cx26 is the connexin protein of 26 kDa, Cx43 is 43 kDa, etc. The principal structural difference between connexin proteins is the length of the C-terminal cytoplasmic tail, with connexin 26 having almost no tail (16 amino acids), while connexins 43 and 32 have long and intermediate ones (73 and 156 amino acids, respectively).

Inherited or acquired alterations in the structure and function of connexin proteins have been linked with various diseases. See, e.g., Delmar, M, Laird, D W, et al. Connexins and Disease, Cold Spring Harb Perspect Biol 10: a029348 (2018); D W Laird and PD Lampe, Cellular mechanisms of connexin-based inherited diseases. Trends in Cell Biology Vol. 32, Issue 1, p58-69 (January 2022).

Research has also associated connexins with assorted conditions and disorders. See, e.g., Willebrords, J, et al., Connexins and their channels in inflammation Crit Rev Biochem Mol Biol. 51 (6): 413-439 (2016); Feng, J, Becker, D L, et al., Connexin 43 upregulation in burns promotes burn conversion through spread of apoptotic death signals, Burns 46 (6): 1389-(September 2020); McDouall, A, Green, C R, et al., Connexins, Pannexins and Gap Junctions in Perinatal Brain Injury. Biomedicines 10:1445 (2022). Connexins have been proposed as therapeutics targets for a number of conditions, including spinal cord injury, perinatal brain injury, nervous system diseases (e.g. Alzheimer's disease, Parkinson's disease), cardiac disorders (e.g. myocardial infarction), ocular disorders (e.g. age-related macular degeneration, diabetic macular edema), acute and chronic wounds (e.g. venous leg ulcers, diabetic foot ulcers), ischemia-reperfusion injury, inflammation, burns and cancer. Reviewed in Laird and Lampe, Therapeutic strategies targeting connexins, Nat Rev Drug Discov. 17 (12): 905-921 (December 2018); Lampe and Laird, Recent advances in connexin gap junction biology, Faculty Reviews 27:11-14 (May 2022). See Becker D L, et al., Translating connexin biology into therapeutics. Semin. Cell Dev. Biol 50, 49-58 (2016). See also the articles in the “Junctional Proteins” issue of FEBS Letters Volume 588, Issue 8, Pages: i, 1185-1490 (Apr. 17, 2014), including Zhang J, et al. Connexin hemichannel induced vascular leak suggests a new paradigm for cancer therapy (p. 1365-1371) and Martin P E, et al., Connexins: Sensors of epidermal integrity that are therapeutic targets (p. 1304-1314). See also, e.g., Van Campenhout R, et al., Mechanisms Underlying Connexin Hemichannel Activation in Disease. Int J Mol Sci. 22 (7): 3503 (April 2021) and U.S. Pat. Nos. 10,401,188 and 11,401,516, issued for “Channel Modulators.”

Strategies for treating eye wounds are reviewed in Ziaei M, et al., Wound healing in the eye: Therapeutic prospects, Advanced Drug Delivery Reviews 126 (2018) 162-176. Ziaei et al. discusses, inter alia, “traditional” treatment strategies including (1) steroids and (2) contact lenses, for example, and a number of “modern” treatment strategies including (3) growth factors (epidermal growth factor, insulin-like growth factor, nerve growth factor and human growth hormone), (4) amniotic membrane-based products, (5) thymosin β4, (6) rebamipide, (7) stem cells and other blood derived products, etc., as well as (8) gap junction hemichannel modulation. See U.S. Pat. No. 8,034,789 issued Oct. 11, 2011, for “Antisense compounds targeted to connexins and methods of use thereof” and Ormonde, S., et al., Regulation of connexin43 gap junction protein triggers vascular recovery and healing in human ocular persistent epithelial defect wounds, J. Membr. Biol. 245 (2012) 381-388 (potential for Cx43 modulation discussed in regard to five compassionate use subjects, but without excluding “the possibility of vehicle effects”). More recently, the use of (9) topical insulin eye drops to treat persistent epithelial defects (PEDs) in the eye that are refractory to usual treatment in clinical practice has been proposed. Diaz-Valle, D, et al., Comparison of the efficacy of topical insulin with autologous serum eye drops in persistent epithelial defects of the cornea. Acta Ophthalmol 2022 June; 100 (4): e912-e919. Others offer the use of (10) punctal plugs, (11) cyanoacrylate glue, (13) debridement, and (14) fibronectin tarsorrhaphy (partial or complete eyelid closure) as possible treatments. See also e.g. Katzman and Jeng, Management strategies for persistent epithelial defects of the cornea. Saudi Journal of Ophthalmology 28:168-172 (2014).

As noted in in Ziaei, et al., many current and emerging therapies proposed for ocular surface wound healing are still largely experimental or conceptual. For example, ocular use of gap junction regulators in humans has not been defined in the clinic, and there are no established doses, dose regimens or methods for the modulation of connexins, connexin gap junctions and/or connexin hemichannels for the treatment of non-healing or persistent ocular or corneal surface defects or disorders.

Such methods, doses and dose regimens are described and claimed herein and provide a long-needed means for treating subjects suffering with a non-healing or persistent ocular surface defect or disorder, and other ocular diseases, disorders and conditions described herein.

In contrast to the methods of treatment described and claimed herein, most current therapies to treat these ocular diseases, disorders and conditions do not treat their cause or causes, which often include uncontrolled inflammation, and have lead to limited success.

### BRIEF SUMMARY

The inventions described and claimed herein have many attributes and embodiments including, but not limited to, those set forth or described or referenced in this Brief Summary. It is not intended to be all-inclusive and the inventions described and claimed herein are not limited to or by the features or embodiments identified in this introduction, which is included for purposes of illustration only and not restriction.

It is an object of the invention to provide methods, doses, dose regimens, compositions and kits for connexin modulation for the treatment of a subject for a non-healing or persistent ocular surface or corneal defect or disorder. In some embodiments, a compound or composition for modulation or inhibition of a connexin, a connexin gap junction and/or a connexin hemichannel is provided for treatment and dosed as described herein. In some embodiments, the non-healing ocular surface defect or disorder is a persistent epithelial defect (PED). In some embodiments, the non-healing ocular surface defect or disorder is a persistent corneal epithelial defect (PCED). In some embodiments, the subject is a human.

In some embodiments, the connexin modulator modulates or inhibits connexin expression. In some embodiments, the connexin modulator inhibits or modulates the expression of connexin 43.

In some embodiments, the connexin modulator is a connexin gap junction modulator. In some embodiments, the connexin gap junction modulator modulates connexin gap junctions.

In some embodiments, the connexin modulator is a connexin hemichannel modulator. In some embodiments, the connexin hemichannel modulator modulates connexin hemichannels. Modulation of a hemichannel may occur by any means. In some embodiments, for example, modulation may occur by inducing or promoting closure of a hemichannel; by preventing, blocking, inhibiting or decreasing hemichannel opening; by suppressing hemichannel permeability; by suppressing ATP release from hemichannels; and/or by triggering, inducing or promoting cellular internalization of a hemichannel and/or gap junction. Hemichannel modulators include blockers and other compounds that interfere with the passage of molecules through a connexin hemichannel. A hemichannel modulator can block or reduce the release of molecules through a hemichannel to an extracellular space, and/or block or reduce the entry of molecules through a hemichannel into an intracellular space. In some embodiments, hemichannel modulators fully or partially block hemichannel opening. In some embodiments, hemichannel modulators fully or partially block, slow or suppress the leak or the passage of molecules through a hemichannel to or from an extracellular space. In some embodiments, hemichannel modulators are compounds that decrease the open probability of a hemichannel.

In some embodiments of the present invention, modulation of gap junctions and hemichannels is accomplished by administration of a connexin expression modulator, a connexin peptidomimetic and/or a small molecule connexin modulator that modulates a corneal epithelial connexin, a corneal epithelial connexin gap junction, or a corneal epithelial connexin hemichannel in a dose regimen disclosed herein. In some embodiments of the present invention, modulation of gap junctions and hemichannels is accomplished by administration of a connexin expression modulator, a connexin peptidomimetic and/or a small molecule connexin modulator that modulates a corneal vascular connexin using a dose regimen disclosed herein. In some embodiments of the present invention, in a dose regimen disclosed herein, modulation of gap junctions and hemichannels is accomplished by administration of a connexin expression modulator, a connexin peptidomimetic and/or a small molecule connexin modulator that modulates (e.g., reduce, slows, inhibits or eliminates) inflammation.

In some embodiments, the connexin modulator, the connexin gap junction modulator and/or the connexin hemichannel modulators used in methods of the invention modulates one or more connexins in the human corneal epithelium, namely, connexin 26, connexin 30, connexin 30.3, connexin 31, connexin 31.1, connexin 32, connexin 43, connexin 45, connexin 50 and connexin 58.

In some embodiments, the connexin modulator comprises an antisense molecule. In some embodiments, the antisense molecule is a connexin43 antisense oligonucleotide. In some embodiments, the connexin modulator may be a connexin 43 antisense polynucleotide comprising, consisting essentially of, or consisting of a sequence according to SEQ ID NOS. 1-and/or modified versions thereof. In some embodiments, antisense oligonucleotide comprises, consists essentially of, or consists of 5′-GTA ATT GCG GCA AGA AGA ATT GTT TCT GTC-3′ (SEQ ID NO:1; lufepirsen). In some embodiments, the antisense oligonucleotide may be chemically modified or may be an unmodified oligonucleotide, e.g. a modified or an unmodified DNA oligonucleotide.

In some embodiments, the connexin modulator comprises a connexin peptidomimetic. In some embodiments, the connexin peptidomimetic is a connexin43 peptidomimetic. In some embodiments, the connexin peptidomimetic comprises, consists essentially of, or consists of SRPTEKT (SEQ ID NO: 101). In some embodiments, the connexin peptidomimetic comprises, consists essentially of, or consists of Gap19, XG19, Gap26 or Gap27. In some embodiments, the connexin peptidomimetic is a connexin26 peptidomimetic (e.g. Gap26 i.e., VCYDKSFPISHVR (SEQ ID NO:102), a connexin32 peptidomimetic (e.g. INCTLQPGCNSV (SEQ ID NO: 103) or ^{37,43}Gap27, which is SRPTEKTIFII (SEQ ID NO: 104) or a connexin45 peptidomimetic, or a connexin50 peptidomimetic (e.g. TAT-Cx50L2, i.e., GGERAPLAADQGSVKKSSSSSKGTKK (SEQ ID NO: 105) or TAT-Cx50CT, i.e., SRARSDDLTV (SEQ ID NO: 106)).

In some embodiments, the connexin modulator comprises a small molecule connexin, connexin gap junction or connexin hemichannel modulator. In some embodiments, the small molecule connexin modulator inhibits or blocks connexin hemichannel opening. In some embodiments, the small molecule connexin modulator moderates ATP release from connexin hemichannels. In some embodiments, the small molecule connexin modulator inhibits or blocks Cx43 hemichannel opening and moderates ATP release from Cx43 hemichannels. In some embodiments, the small molecule connexin modulator is a compound according to Formula I (e.g. tonabersat). In some embodiments, the small molecule connexin modulator is a tonabersat prodrug compound according to Formula II.

In some embodiments, the invention relates to methods for the modulation of a connexins, gap junction channels and/or hemichannels using a therapeutically effective amount of a connexin modulator in a dose regimen described herein. In some embodiments, a therapeutically effective amount of a connexin antisense compound (e.g. lufepirsen) is administered. In some embodiments, a therapeutically effective amount of a connexin peptidomimetic compound (e.g. Peptide5, Gap19, XG19, CXT 1 to CXT 5, Antp/CXT 1 to Antp/CXT 5, etc.) is administered. In some embodiments, a therapeutically effective amount of a small molecule connexin hemichannel blocker compound (e.g. tonabersat or a tonabersat prodrug) is administered.

In some embodiments, the invention relates to methods for the modulation of a connexins, gap junction channels and/or hemichannels using new particular doses and newly discovered and clinically confirmed dose regimens of connexin modulators, gap junction modulators and/or hemichannel modulators for ocular surface recovery in a subject with a non-healing or persistent ocular surface or corneal defect or disorder, or both. Non-healing ocular surface or corneal defect or disorders treated using the doses, compositions, dose regimens and methods of the invention include persistent epithelial defects (PED) and persistent corneal epithelial defect (PCED). They include PEDs and PCEDs from any cause including chemical injury, thermal injury, disease and inflammation.

In some embodiments, the non-healing ocular surface defect or disorder results from a chemical and/or thermal injury. In some embodiments, the non-healing ocular surface defect or disorder results from a physical trauma or injury. In certain embodiments, the non-healing ocular surface defect or disorder is a PED or PCED resulting from a chemical and/or thermal injury. In some embodiments, the chemical and/or thermal injury resulting in a PED or PCED is a severe chemical and/or thermal injury. In some embodiments, severe ocular surface and/or corneal burns and injuries are those with greater than 50% conjunctival or >6 clock hours of limbal involvement. These include ocular surface and corneal burns and injuries ≥3 on the Dua Classification scale. In some embodiments, the non-healing ocular surface or corneal defect or disorder results from inflammation, or from an inflammatory disease. In some embodiments, the non-healing ocular surface or corneal defect or disorder results from inflammation of the eye. In some embodiments, the non-healing ocular surface or corneal defect or disorder results from an inflammatory disease of the eye, the ocular surface, or the cornea.

All PED and PCED etiologies, no matter the cause, may be treated using the methods and compositions of the invention as described herein. PED and PCED etiologies include not only inflammation and inflammatory disease, neurotrophic disease and neurotrophic keratitis, but chemical, mechanical, thermal causes, and epithelial/limbal stem cell deficiency. In some embodiments, methods, compositions and/or doses of the invention are used to treat PED and/or PCED caused by or resulting from chemical injury. In some embodiments, methods, compositions and/or doses of the invention are used to treat PED and/or PCED caused by or resulting from thermal injury. In some embodiments, methods and/or doses of the invention are used to treat PED and/or PCED caused by or resulting from mechanical injury. In some embodiments, methods and/or doses of the invention are used to treat PED and/or PCED caused by or resulting from inflammation or an inflammatory disease. In some embodiments, methods, compositions and/or doses of the invention are used to treat PED and/or PCED caused by or resulting from neurotrophic disease (e.g. neurotrophic keratitis).

In some embodiments, the non-healing ocular surface defect or disorder is an ocular surface ulcer. In some embodiments, the non-healing ocular surface defect or disorder is a corneal ulcer. In some embodiments, the ocular surface ulcer or the corneal ulcer is caused by a bacterium, a virus, a fungus or a parasite. In some embodiments, the ocular surface ulcer or the ocular surface ulcer or the corneal ulcer results from acanthamoeba keratitis, fungal keratitis or herpes simplex keratitis. In other embodiments, the ocular surface ulcer or the corneal ulcer is caused by a trauma such as an abrasion (e.g. scratch or cut) or a foreign body in the eye. In another embodiment, the ocular surface ulcer or the corneal ulcer is caused by allergic eye disease. In another embodiment, the ocular surface ulcer or the corneal ulcer is caused by dry eye. In some embodiments, the non-healing ocular surface ulcer or corneal ulcer is caused by or results from inflammation or an inflammatory disease, disorder, or condition. In some embodiments, the ocular surface ulcer or the corneal ulcer results from another cause (e.g. an injury or burn).

In some embodiments of the methods described and claimed herein, a connexin inhibitor or modulator is administered to subject having a non-healing surface defect or disorder on Day 1, Day 2 and on about Day 14. In some embodiments, a connexin inhibitor or modulator is administered to the subject again on about Day 28. In some embodiments, a connexin inhibitor or modulator may be administered to a subject having a non-healing surface defect or disorder again on about Day 35. In some embodiments, a connexin inhibitor or modulator may also be administered to a subject on Day 7 and/or Day 21. In some embodiments, the non-healing surface defect or disorder to be treated is a PED. In some embodiments, the non-healing surface defect or disorder to be treated is a PCED. In some embodiments, the PED or PCED results from a chemical and/or thermal injury, or from inflammation.

In some embodiments, a single dose of a connexin modulator is administered in a regimen for treating a non-healing surface defect or disorder as described and claimed herein (e.g., single doses administered on Days, 1, 2 and 14, and so on, including in other dose regimens described herein). In some embodiments, the daily dose of a connexin modulator for administration in a dose regimen of the present invention is administered in divided amounts (e.g. one-half dose, administered to the subject twice per day on Days, 1, 2 and 14, etc.). In some embodiments, a single dose or divided dose of a connexin modulator is administered on one or more or all of days in each of the dose regimens described herein.

In some embodiments of the present invention, the method for treating a subject for a non-healing ocular surface defect comprises administering to the subject a therapeutically effective amount of a connexin modulator, wherein the connexin modulator is administered at least once on Day 1, at least once on Day 2, and at least once at about Day 14. In some embodiments, the connexin modulator is a connexin 43 modulator. In some embodiments, the connexin 43 modulator modulates connexin 43 expression. In some embodiments, the connexin expression modulator is an antisense molecule. In some embodiments, the connexin 43 antisense molecule is lufepirsen. In some embodiments, lufepirsen is administered in a composition comprising a nonionic polyoxyethylene-polyoxypropylene block co-polymer carrier (e.g. Pluronic F-127). In some embodiments, lufepirsen is administered in a composition comprising a cellulose-based carrier, e.g., hydroxyethylcellulose, carboxymethylcellulose hydroxymethylcellulose, hydroxypropylmethylcellulose, etc,. or other pharmaceutically acceptable carrier.

In some embodiments, from 3 to 4 doses of a connexin modulator, e.g. lufepirsen, are administered at Days 1, 2 and 14 (and, optionally, on or about Day 28). In other embodiments, 4 to 5 doses are administered, with a dose or doses of a connexin modulator, e.g. lufepirsen or another connexin antisense or modulator, also administered at about Day 35, in addition to Days 1, 2 and 14 (or in addition to Days 1, 2, 14 and, optionally, about Day 28). In some embodiments of these dose regimens, doses of a connexin modulator, e.g. lufepirsen, may also be administered at about Day 7 and/or Day 21 In some embodiments, connexin modulator doses, e.g. lufepirsen, are administered on Days, 1, 2, 14 and 28.

In some embodiments, connexin modulator doses, e.g. lufepirsen, are administered on Days 1, 2, 14 and 21.

In some embodiments, connexin modulator doses, e.g. lufepirsen, are administered on Days 1, 2, 7, 14 and 21.

In some embodiments, connexin modulator doses, e.g. lufepirsen, are administered on Days 1, 2, 7, 14, 21 and 28.

In some embodiments, connexin modulator doses, e.g. lufepirsen, are administered on Days 1, 2, 14, 28 and 35. In some embodiments, connexin modulator doses, e.g. lufepirsen, are administered on Days 1, 2, 7, 14, 28 and 35. In some embodiments, connexin modulator doses, e.g. lufepirsen, are administered on Days 1, 2, 7, 14, 21, 28 and 35. In some embodiments, connexin modulator doses, e.g. lufepirsen, are administered on Days, 1, 2, 7, 14, 21, 28, and 35. In some embodiments, a dose is also administered in any of these dosing regimens after Day 35. In some embodiments, administration is to a PED or PCED. In some embodiments, the connexin modulator comprises a connexin antisense molecule (e.g. lufepirsen). In some embodiments, the connexin modulator comprises a connexin peptidomimetic (e.g., Peptide5, Gap19, XG19, aCT1, etc.). In some embodiments, the connexin modulator comprises a small molecule connexin hemichannel blocker (e.g., tonabersat). In some embodiments, the connexin modulator is a connexin 43 modulator. In some embodiments, administration is to a PED or PCED. The connexin modulator may be administered in a single or divided dose on any dosing days.

In some embodiments, 3 doses of a connexin modulator (e.g. lufepirsen) are administered over 14 days to treat a non-healing or persistent ocular or corneal surface defect in a subject, including, for example, on Days 1 and 2. In some embodiments, 4 doses of a connexin modulator (e.g. lufepirsen) are administered over about 28 days. In some embodiments, 5 doses of a connexin modulator (e.g. lufepirsen) are administered over about to about 35 days. In some embodiments, 6-7 doses of a connexin modulator (e.g. lufepirsen) are administered over about 35 days. For example, in one embodiment, a connexin modulator (e.g. lufepirsen) is administered 4 times over Days 1 and 2, on about Day 14 and 28. In another embodiment, a connexin modulator (e.g. lufepirsen) is administered 5 times over Days 1 and 2, on about Day 14, on about Day 28 and on about Day 35. In another embodiment, a connexin modulator (e.g. lufepirsen) is administered 5 times over Days 1 and 2, on about Day 7, on about Day 14 and on about Day 28. In another embodiment, a connexin modulator (e.g. lufepirsen) is administered 6 times over Days 1 and 2, on about Day 7, on about Day 14, on about Day 21 and on about Day 28. In another embodiment, a connexin modulator (e.g. lufepirsen) is administered 7 times over Days 1 and 2, on about Day 7, on about Day 14, on about Day 21 and on about Day 28. Reference to “administration” here refers, of course, to administration of a connexin modulator in a single dose or in divided doses. In some embodiments of the present invention, modulation of connexins, gap junctions and/or hemichannels is accomplished by administration of a connexin antisense oligonucleotide (e.g. lufepirsen) to the eye of a subject having a non-healing or persistent ocular surface or corneal defect or disorder (e.g. a PED or a PCED).

In some embodiments, the amount of connexin modulator administered to a subject with a non-healing ocular surface defect or disorder (e.g. a PED) or non-healing corneal surface defect or disorder (e.g. a PCED) in any of the dosing schedules described herein (e.g. Days 1, and 14; Days 1, 2, 14 and, optionally, Day 28; Days 1, 2, 7, 14, 21, and, optionally, Day 28; and so on) is a therapeutically effective amount.

In some embodiments, the amount of connexin modulator administered to a subject with a non-healing ocular surface defect or disorder (e.g. a PED) or non-healing corneal surface defect or disorder (e.g. a PCED) in any of the dosing schedules described herein is one or more of the dose amounts described herein (e.g. milligram per milliliter (mg/mL) connexin modulator dosing), micromolar (μM) connexin modulator concentration dosing, milligram (mg) connexin modulator dosing, etc.). The particular doses described herein may be administered to a subject with a non-healing ocular surface defect or disorder (e.g. a PED) or non-healing corneal surface defect or disorder (e.g. a PCED), for example, on Days 1, 2, 14 and, optionally, Day 28 and/or Day 35; on Days 1, 2, 14 and optionally on Days 7, 21 and/or 28; etc., or any other dose regimen described herein.

In some embodiments of the method of the present invention, the connexin modulator administered to the non-healing surface defect or disorder in a subject is 0.06 wt. % lufepirsen. In some embodiments, the connexin modulator administered to a subject with a non-healing surface defect or disorder is 0.006 wt. % lufepirsen. In some embodiments, the connexin modulator administered is at least about 0.06 wt. % lufepirsen. In some embodiments, the connexin modulator administered to a subject with a non-healing surface defect or disorder is at least about 0.006 wt. % lufepirsen. In some embodiments, administration is to a PED or PCED. In some embodiments, a composition comprising or consisting essentially of a pharmaceutically acceptable carrier and about 0.06 wt. % lufepirsen, about 0.006 wt. % lufepirsen, at least about 0.06 wt. % lufepirsen or least about 0.006 wt. % lufepirsen is administered to heal the non-healing surface defect or disorder in a subject.

In some embodiments of the present invention, the non-healing surface defect or disorder is treated with composition comprising 0.6 mg/mL lufepirsen. In some embodiments of the present invention, the non-healing surface defect or disorder is treated with composition comprising 0.06 mg/mL lufepirsen. In some embodiments, the composition comprises at least about 0.6 mg/mL lufepirsen. In some embodiments, the composition comprises at least about 0.06 mg/mL lufepirsen. In some embodiments, administration is to a PED or PCED.

In some embodiments of the present invention, the non-healing surface defect or disorder in a subject is treated with a composition comprising or consisting essentially of about 0.18 mg of lufepirsen in the regimens described herein. In some embodiments, each dose of lufepirsen applied to the non-healing surface defect or disorder in or on the subject comprises or consists essentially of at least about 0.18 mg of lufepirsen in the regimens described herein. In some embodiments of the present invention, the non-healing surface defect or disorder is treated with composition comprising or consisting essentially of about 0.018 mg of lufepirsen in the regimens described herein. In some embodiments, each dose of lufepirsen applied to the non-healing surface defect or disorder comprises or consists essentially of at least about 0.018 mg of lufepirsen in the regimens described herein. In some embodiments, administration is to a PED or PCED.

In some embodiments, about 0.18 mg of lufepirsen (or other connexin antisense or connexin modulator), about 0.018 mg of lufepirsen (or other connexin antisense or connexin modulator), or from about 0.1 mg, 0.2 mg, 0.3 mg, 0.4 mg or about 0.5 mg to about 1 mg of lufepirsen (or another connexin antisense, or other connexin modulator) is administered to the non-healing or persistent ocular surface or corneal defect or disorder in a single dose, or in two or more divided doses, e.g., for twice daily administration in up to 6 doses administered on Days 1, 2 and 14. In other embodiments, at least about 0.1 mg, 0.2 mg, 0.3 mg, 0.4 mg or 0.5 mg or at least about 1 mg of lufepirsen (or another connexin antisense, or other connexin modulator) is administered in a single dose, or in two or more divided doses to the non-healing surface defect or disorder, e.g., for twice daily administration in up to 6 to 10 doses administered on Days 1, 2 and 14, or using other dose regimen described herein that includes Day 28 and/or Day 35. In other embodiments, at least about 0.5 mg or at least about 1 mg of lufepirsen (or another connexin antisense, or other connexin modulator) is administered in single or divided doses, e.g., for twice daily administration, in up to 14 doses administered on Days 1, 2 and 14, and one or more of Day 7, Day 21, Day 28 and/or Day 35, for example. In some embodiments, administration is to a PED or PCED in a subject. In some embodiments, the connexin antisense administered to the subject in the amounts described herein comprises or consists essentially of an antisense compound having a sequence according to SEQ ID NOS: 2-16. In some embodiments, the connexin antisense administered to the subject in the amounts described herein on the days described herein, comprises or consists essentially of another connexin 43 antisense compound.

In any of the embodiments of these doses and dosing regimens and methods, the lufepirsen doses may be administrated to a PED or PCED in a subject. In some embodiments of these doses and dosing regimens and methods, the connexin modulator dose administered comprises another connexin antisense or modulator, e.g., another Cx43 antisense or Cx43 modulator other than or in addition to lufepirsen, or a modulator of another ocular or corneal epithelial connexin other than or in addition to Cx43. In some embodiments, the connexin antisense administered to the subject in the amounts described herein on the days described herein, comprises or consists essentially of an antisense compound for modulating expression of one or more connexins selected from the group consisting of connexin 26, connexin 30, connexin 30.3, connexin 31, connexin 31.1, connexin 32, connexin 43, connexin 45, connexin and connexin 58.

In some embodiments of the present invention, the volume of a composition comprising or consisting essentially of a connexin modulator administered a non-healing surface defect or disorder in a subject is about 300 μL. In other embodiments, the volume of the composition administered is from about 250 μL to about 1.0 mL. In some embodiments, the composition comprises about 0.01 mg, 0.018 mg, from about 0.18 mg to about 0.2 mg of lufepirsen, or from about 0.18 mg or 0.2 mg or 0.5 mg to about 1 mg of lufepirsen. In some embodiments, the composition comprises at least about 0.018 mg lufepirsen, at least about 0.18 mg of lufepirsen, at least about 0.5 mg of lufepirsen or at least about 1.0 mg of lufepirsen. In some embodiments, instead of or in addition to lufepirsen, the composition comprises about 0.01 mg, about 0.018 mg, from about 0.18 mg to about 0.2 mg, or from about 0.18 mg or 0.2 mg or 0.5 mg to about 1 mg of another connexin 43 antisense compound (or other connexin modulator, including, for example, other antisense, peptidomimetic, small molecule connexin modulators that modulate connexin 43 or another ocular or corneal epithelial connexin(s)). In some embodiments, instead of or in addition to lufepirsen, the composition comprises at least about 0.018 mg, at least about 0.18 mg of, at least about 0.5 mg or at least about 1.0 mg of another connexin antisense or other connexin modulator (e.g. other antisense, peptidomimetic and/or small molecule connexin modulators that modulate connexin 43 or other ocular or corneal epithelial connexin(s)).

In some embodiments, the composition for use in the methods of the invention comprises a reverse thermosetting gel. In some embodiments, the composition for use in the methods of the invention comprises a pluronic gel, also known as a poloxamer gel. In some embodiments, the composition for use in the methods of the invention comprises poloxamer F-127 (poloxamer 407 or Pluronic F-127). In some embodiments, the composition for use in the methods of the invention comprises about 20-30 mg/mL of a poloxamer F-127. In some embodiments, the composition for use in the methods of the invention comprises about 22-25 mg/mL of a poloxamer F-127. In some embodiments, the composition for use in the methods of the invention comprises about 22.6 mg/mL of a poloxamer F-127.

In some embodiments, the connexin modulator is lufepirsen in a composition comprising Pluronic F-127, a buffer or buffers and water for injection. Buffers incorporated in the lufepirsen composition may comprise sodium phosphate dibasic heptahydrate or potassium phosphate (monobasic) or both. In some embodiments, the composition comprises 0.06% or 0.006% lufepirsen, at least about 0.06% or at least about 0.006% lufepirsen. In some embodiments, the buffered composition comprises 0.6 mg/mL or 0.06 mg/mL lufepirsen, at least about 0.6 mg/mL or at least about 0.06 mg/mL lufepirsen. In some embodiments, about 300 mL of the lufepirsen composition is administered to a non-healing surface disorder on Day 1, Day 2 and on Day 14. If necessary or desired, in some embodiments, about 300 μL of a lufepirsen dose is administered to a subject having a non-healing surface disorder again on about Day 28. If necessary or desired, in some embodiments, about 300 μL of a lufepirsen dose is administered to a subject having a non-healing surface disorder again on about Day 35. In other embodiments, if deemed necessary or desired, about 300 μL of the lufepirsen composition is also administered to a subject having a non-healing surface disorder again on Day 7 and/or on about Day 21. In some embodiments, the non-healing surface disorder treated with the composition is a PED or PCED. In some embodiments, the PED or PCED results from a chemical and/or thermal injury or other trauma. In some embodiments, from about 300 μL to 1.0 mL of the lufepirsen composition is administered to the non-healing surface disorder. In some embodiments, the connexin modulator administered in this composition comprises another connexin 43 antisense compound. In some embodiments, the connexin modulator administered in this composition comprises an antisense compound against another ocular or corneal epithelial connexin. In some embodiments, the connexin modulator administered in this composition comprises another connexin 43 antisense compound, an antisense compound against another ocular or corneal epithelial connexin, and/or another connexin modulator (e.g. a peptidomimetic or small molecule connexin modulator).

In some embodiments of the present invention, modulation of gap junctions and hemichannels is accomplished by administration of a small molecule hemichannel blocker that blocks or modulates opening of a corneal epithelial connexin hemichannel. In some embodiments, treatment of a non-healing ocular surface defect by modulation of a corneal epithelial hemichannel is accomplished by topical, oral or systemic administration of a small molecule hemichannel blocker. In some embodiments, a therapeutically effective amount of the small molecule is administered in a dose regimen described herein. In some embodiments, the dose of the small molecule is administered in a dose regimen described herein is one or more of the doses set forth herein. In some embodiments, the small molecule hemichannel blocker is tonabersat.

In any embodiment of the invention, the administration of the connexin modulator may be topical. In some embodiments, a connexin antisense connexin modulator (e.g. lufepirsen) is administered topically. In some embodiments, a connexin peptidomimetic connexin modulator (e.g., XG19) is administered topically. In some embodiments, a small molecule connexin modulator (e.g. tonabersat) is administered topically. In some embodiments, a combination of one or more compounds selected from the group consisting of connexin antisense connexin modulators (e.g. lufepirsen), connexin peptidomimetic connexin modulators (e.g., XG19), and small molecule connexin modulators (e.g. tonabersat) is administered topically. In some embodiments, one or more compounds selected from the group consisting of a connexin antisense connexin modulators (e.g. lufepirsen), connexin peptidomimetic connexin modulators (e.g., XG19), and small molecule connexin modulators (e.g. tonabersat) and another therapeutic agent or agents is/are administered topically. In some embodiments, the other therapeutic agent or agents is selected from the group consising of antibiotics, antivirals, anti-parasitics, antifungals, steroids, anti-inflammatoires and immunosuppressants. In some embodiments, the other therapeutic agent or agents is selected from the group consising of pannexin 1 (Panx1) inhibitors (e.g. probenecid, ¹⁰Panx1), inhibitors of its associated P2X receptors (and any of its isoforms), and inhibitors of Panx1-P2X signalosomes. In some embodiments, one or more compounds selected selected from the group consisting of of pannexin 1 (Panx1) inhibitors (e.g. probenecid), inhibitors of its associated P2X receptors (and any of its isoforms), and inhibitors of Panx1-P2X signalosomesis is administered (e.g. topically), alone or together with a connexin modulator or another therapeutic agent. Other useful pannexin inhibitors are described in U.S. Pat. No. 10,465,188 (“Channel Modulators”).

In some embodiments, the subject has an amniotic membrane affixed to the surface of the eye to be treated. In some embodiments, the amniotic membrane is a permanent surgical graft. In some embodiments, the amniotic membrane is a dehydrated sutureless graft. In some embodiments, the amniotic membrane is a cryopreserved sutureless graft.

In some embodiments, a connexin modulator, gap junction modulator and/or hemichannel modulator is administered to the subject under an amniotic membrane. In some embodiments, an amniotic membrane is affixed to the eye of the subject after application of the modulator to the non-healing surface defect or disorder.

In some embodiments, a connexin modulator, a gap junction modulator and/or a hemichannel modulator is/are administered to the subject under a contact lens. In some embodiments, the contact lens is applied to the eye of the subject at about the same time as, immediately after or sometime following administration of the connexin modulator, a gap junction modulator and/or a hemichannel modulator. In some embodiments, the contact lens is a silicon hydrogel contact lens. In some embodiments, the lens is loaded with a connexin modulator, a gap junction modulator and/or a hemichannel modulator prior to application of the lens to the non-healing surface defect or disorder in or on the eye of the subject. In some embodiments, the modulator is lufepirsen.

In some embodiments, the contact lens is a therapeutic contact bandage lens. Therapeutic contact bandage lenses are also referred to as “bandage lenses” and “therapeutic bandage lenses.” In some embodiments, the therapeutic bandage lens is, for example, a soft contact lens, a gas permeable (scleral) lens or a 3D-printed bio-gel. Some suitable FDA-approved soft bandage lenses include Acuvue Oasys with Hydraclear Plus (Johnson & Johnson Vision), Air Optix Night & Day Aqua (Alcon), PureVision (Bausch+Lomb) and UCL 55% (United Contact Lens) and Kontur (Kontur Kontact Lens).

In some embodiments, the present invention relates to methods for the modulation of a connexin, a connexin gap junction channel and/or a connexin hemichannel using doses and dose regimens of connexin modulators (including e.g. gap junction modulators and/or hemichannel modulators) for ocular surface recovery and/or corneal epithelial recovery. In some embodiments, modulation of gap junctions and hemichannels is accomplished by administration of a connexin 43 antisense oligonucleotide, a connexin 43 peptidomimetic and/or a small molecule connexin 43 hemichannel blocker. In other embodiments, connexin 26, connexin 30, connexin 30.3, connexin 31, connexin 31.1, connexin 32, connexin 45, connexin 50 and/or connexin 58 modulators are used, alone or in combination with or in conjunction with a connexin 43 modulator.

In some embodiments, a gap junction modulator or hemichannel modulator (e.g. lufepirsen) is applied topically to the eye of a subject with a non-healing or persistent ocular surface and/or corneal epithelial disorder and another topically administered gap junction modulator (e.g. Peptide5, Ga19, XG19, or another peptidomimetic) or topically, orally or systemically dispensed hemichannel modulator (e.g. tonabersat, or another orally or systemically bioavailable gap junction and/or hemichannel modulator) is administered to the subject at the same time as, or before or after the topically applied gap junction modulator or hemichannel modulator.

In some embodiments, the method comprises co-administering a topical connexin modulator and/or a gap junction modulator and a systemically available connexin hemichannel modulator. The co-administration of the gap junction channel modulator can be simultaneously with, subsequent to, or before the administration of the connexin hemichannel modulator. In some embodiments, compounds of Formula I, for example tonabersat, compounds of Formula II and/or a peptidomimetic (e.g. Peptide5, XG19, etc.), may be co-administered with a connexin expression modulator, e.g., lufepirsen, or anti-connexin peptidomimetic.

In some embodiments, the invention provides the use of a connexin modulator in the manufacture of a medicament for treatment of a non-healing or persistent ocular surface and/or corneal defect or disorder modulation. In some embodiments, the medicament comprises or consists essentially of a connexin antisense molecule, e.g. lufepirsen, including in the amounts and/or concentrations described herein. In some embodiments, the medicament comprises or consists essentially of a connexin peptidomimetic, e.g. Peptide5, Gap19, XG19, an aCT1 peptide (e.g., CXT 1, CXT, CXT 3, CXT 4, CXT 5, or any other C-terminal connexin peptidomimetic, with or without a C-terminally or N-terminally attached cell-penetrating peptide), etc. In some embodiments, the medicament comprises or consists essentially of a small molecule connexin hemichannel blocker, e.g. a compound according to Formula I, including tonabersat, carabersat, and prodrugs thereof, including tonabersat prodrugs according to Formula II.

In some embodiments, connexin modulators and connexin43 modulators include means for down-regulating the connexin transcription or translation of a connexin, such as antisense molecules, for example. Means for connexin modulation also include ZO-1 binding peptides in the case of connexin 43 (e.g. aCT1, αCT1, CXT 1, CXT 2, CXT 3, CXT 4, CXT 5, etc.) for example. Preferred gap junction channel modulators are connexin43 expression modulators, connexin43 gap junction modulators and connexin 43 hemichannel modulators. Especially preferred connexin 43 modulators include lufepirsen, XG19 and tonabersat.

In some embodiments, the invention relates to pharmaceutical compositions and articles of manufacture, including kits with instructions for use, comprising a therapeutically effective amount of a connexin modulator, and dose regimens for treating non-healing ocular and corneal surface defects and disorders with one or more connexin modulators, including connexin expression modulators, gap junction modulators and/or hemichannel modulators. In some embodiments, the kit includes or is associated with instructions (e.g. online instructions) for dosing and treating PED and/or a PCED as described herein.

In some embodiments, the connexin modulators (e.g. connexin expression modulators, connexin gap junction modulators and connexin hemichannel modulators, including, for example, modulators of Cx26, Cx30, Cx30.3, Cx31.1, Cx32, Cx36, Cx37, Cx40, Cx43, Cx45, Cx50 or Cx58 connexins, gap junctions and/or hemichannels) are combined with a pharmaceutically acceptable carrier or diluent to produce a pharmaceutical composition. In some embodiments, suitable carriers and diluents include buffered, aqueous solutions, isotonic saline solutions, for example phosphate-buffered saline, isotonic water, and the like. In some embodiments, the carrier is a pluronic or poloxamer gel. In some embodiments, the gel is pluronic F-127. In some embodiments, the pharmaceutical composition comprises a buffer. In some embodiments, the buffer comprises or consists essentially of sodium phosphate dibasic heptahydrate or sodium phosphate monobasic or both.

In some embodiments, methods of the invention, the doses and the dose regimens, and the connexin modulators (e.g. connexin expression modulators, connexin gap junction modulators and connexin hemichannel modulators, including, for example, modulators of Cx26, Cx30, Cx30.3, Cx31.1, Cx32, Cx36, Cx37, Cx40, Cx43, Cx45, Cx50 or Cx58 connexins, gap junctions and/or hemichannels) described herein are used for the treatment of a subject for uveitis, which includes inflammation of the middle layer of the eye, the uvea, and surrounding tissue. In some embodiments, connexin 43 modulators are used to treat one or more forms of uveitis. In some embodiments, other connexin modulators are used, including, for example, connexin 26 modulators, connexin 45 modulators or others, alone or in combination. In some embodiemts, anterior uveitis is treated. In some embodiemts, intermediate uveitis is treated. In some embodiments, posterior uveitis is treated. In some embodiments, panuveitis is treated. In some embodiments, the uveits to be treated results from any cause, or is multifactorial. In each of these embodiments, one or more symptoms of uveitis are treated or are alleviated, in whole or in part, using methods of the invention, the doses and the dose regimens, and the connexin modulators as described herein.

In some embodiments, methods of the invention, any of the doses and the dose regimens, and any the connexin modulators (e.g. connexin expression modulators, connexin gap junction modulators and connexin hemichannel modulators, including, for example, modulators of Cx26, Cx30, Cx30.3, Cx31.1, Cx32, Cx36, Cx37, Cx40, Cx43, Cx45, Cx50 or Cx58 connexins, gap junctions and/or hemichannels) described herein are used for the treatment of a subject for blepharitis.

In some embodiments, methods of the invention, the doses and the dose regimens, and the connexin modulators (e.g. connexin expression modulators, connexin gap junction modulators and connexin hemichannel modulators, including, for example, modulators of Cx26, Cx30, Cx30.3, Cx31.1, Cx32, Cx36, Cx37, Cx40, Cx43, Cx45, Cx50 or Cx58 connexins, gap junctions and/or hemichannels) described or referenced herein are used for the treatment of a subject with any stage of dry eye disease (DED), also known as dry eye syndrome (DES), keratoconjunctivitis sicca (KCS), and keratitis sicca.

In other embodiments, methods of the invention, the doses and the dose regimens, and the connexin modulators described or referenced herein are used for the treatment of a subject for Sjögren's syndrome-associated KCS and one or more symptoms of Sjögren's syndrome-associated KCS are treated or are alleviated, in whole or in part.

In other embodiments, methods of the invention, the doses and the dose regimens, and the connexin modulators described or referenced herein are used for the treatment of a subject for Fuchs' dystrophy, and the treatment results in the alleviation of one or more symptoms of Fuchs' dystrophy, in whole or in part.

In some embodiments, in addition use of the present invention for ocular surface recovery and/or corneal epithelial recovery, the present invention relates to methods for the modulation of a connexin, a connexin gap junction channel and/or a connexin hemichannel using doses and dose regimens of connexin modulators (including e.g. gap junction modulators and/or hemichannel modulators) for uvea recovery, sclera recovery, recovery of tear glands and tear ducts, recovery of meibomian glands, eyelid recovery, etc. In some embodiments, modulation of gap junctions and hemichannels is accomplished by administration of a connexin antisense oligonucleotide, a connexin 43 peptidomimetic and/or a small molecule connexin hemichannel blocker. In other embodiments, connexin 26, connexin 30, connexin 30.3, connexin 31, connexin 31.1, connexin 32, connexin 45, connexin 50 and/or connexin 58 modulators are used, alone or in combination with or in conjunction with a connexin 43 modulator and/or another therapeutic agent.

In some embodiments, doses of the connexin modulators described or referenced herein are administered with, or co-formulated for administration to a subject with, effective amounts of one or more compounds selected from the group consisting of anti-microbial agents, antibiotics, antivirals, anti-fungals, anti-parasitics, dilating eye drops, steroidal anti-inflammatories, immunosuppressants, immunomodulatory agents, dry eye treatment agents (e.g. Tyrvaya, Rstasis, Cequa, Xiidra, Eysuvis), artificial tear compositions, and lubricating agents.

In some embodiments, one or more of the connexin modulators described or referenced herein are formulated with one more anti-microbial agents for use in the methods and regimens described herein.

In some embodiments, one or more of the connexin modulators described or referenced herein are formulated with one more anti-bacterial agents for use in the methods and regimens described herein.

In some embodiments, one or more of the connexin modulators described or referenced herein are formulated with one more anti-parasitic agents for use in the methods and regimens described herein.

In some embodiments, one or more of the connexin modulators described or referenced herein are formulated with one more anti-fungal agents for use in the methods and regimens described herein.

In some embodiments, one or more of the connexin modulators described or referenced herein are formulated with one more anti-viral agents for use in the methods and regimens described herein.

### BRIEF DESCRIPTION OF THE FIGURES

FIG. 1 shows the dose regimen from a connexin modulator clinical trial for the treatment of ocular surface defects and disorders comprising a non-healing persistent corneal epithelial defect.

### DETAILED DISCLOSURE

In some aspects, inventions described and claimed herein relate to modulators of connexins, connexin gap junctions, and connexin hemichannels, and their use in methods for the treatment of non-healing ocular defects and disorders, including non-healing corneal defects and disorders, and doses and dose regimens therefor.

In some embodiments, inventions described and claimed herein relate to modulators of connexins, connexin gap junctions, and connexin hemichannels, and their use in methods for the treatment of other ocular defects, diseases, disorders and conditions, including uveitis, blepharitis, Sjögren's syndrome-associated keratoconjunctivitis sicca, Fuchs' dystrophy, and all forms of dry eye disease.

In order to maintain a smooth optical surface, the corneal epithelium has to continuously renew itself so as to maintain its function as a barrier to fluctuating external surroundings and various environmental insults. After trauma, the cornea typically re-epithelializes promptly thereby minimizing the risk of infection, opacification, or perforation. A persistent epithelial defect or persistent corneal epithelial defect is usually referred to as an ocular or corneal epithelial lesion that does not heal after approximately two weeks of treatment with standard therapies. They can occur, for example, following exposure to toxic agents, chemicals, mechanical injury, thermal insult, and ocular surface infections and are associated with significant clinical morbidity in patients, resulting in discomfort or visual loss. The major risk factor for most non-healing ocular surface defect or disorder is vision loss.

In one aspect this invention relates to pharmaceutical compositions, articles of manufacture, kits and methods for treating a subject having (or at risk for having) a non-healing or persistent ocular surface or corneal defect or disorder, for example, an ocular persistent epithelial defect or a persistent corneal epithelial defect, by administering a therapeutically effective amount of at least one connexin modulator to the eye of said subject using new, clinically validated dosing regimens. Therapeutically effective doses may be used with these new dosing regimens. New, clinically validated doses may be used with these new dosing regimens. These doses used with these dosing regimens proved for durable closure of non-healing ocular surface or corneal defects or disorders, including, for example, ocular persistent epithelial defects and persistent corneal epithelial defects.

In one embodiment, a composition comprising a connexin modulator is applied topically to the eye of the subject. In one embodiment, the connexin modulator is administered topically to the eye of the subject followed by application of a bandage contact lens, and/or an amniotic membrane. In some embodiments, the connexin modulator is a connexin 43 modulator. In some embodiments, the connexin 43 modulator modulates connexin 43 expression. In some embodiments, the connexin modulator (e.g. a connexin 43 modulator) is placed into a bandage contact lens which is then placed on the eye of the subject.

In some embodiments of the methods of this invention, a non-healing ocular surface defect or disorder is treated by pulsed application of a connexin modulator in a dosing regimen as described herein (e.g. three to seven times over 14 to about 35 days). In some embodiments, a connexin modulator is administered. In some embodiments, a connexin gap junction modulator is administered. In some embodiments, a connexin hemichannel blocker or modulator is administered. In some embodiments, the connexin modulator blocks or modulates connexin protein expression (e.g. connexin 43 protein expression). In some embodiments, the connexin gap junction modulator and/or the connexin hemichannel blocker or modulator blocks or modulates connexin 43 gap junctions and/or connexin 43 hemichannels.

In some embodiments, the inventions relate to pharmaceutical compositions, articles of manufacture, and methods for treating non-healing ocular surface defects or disorders by administering a therapeutically effective amount of at least one connexin modulator to the eye of said subject, and/or orally or systemically, for example, in the case of non-topically administered connexin modulators. In some embodiments, more than one connexin modulator is administered to the eye of said subject, and/or orally or systemically.

In some embodiments the non-healing ocular surface defect or disorder is a persistent epithelial defect (PED). In some embodiments the non-healing ocular surface defect or disorder is a persistent corneal epithelial defect (PCED). In methods of the invention, administering dose described herein, or another therapeutically effective amount, of at least one connexin modulator in pulse-dose format, with administrations on Day 1 and Day 2, followed by administration on Day 14 and optionally on or on about Day 28 (and also optionally administration on or on about Day 35) is effective for healing a persistent or non-healing ocular surface or corneal epithelial defect or disorder. See Example 2, below. Connexin modulator doses may also be administered on Day 7 and/or Day 21. In some embodiments, the connexin modulator is a connexin 43 modulator (e.g. a connexin expression modulator, such as an antisense connexin expression modulator, including a Cx43 antisense). Other embodiments include other connexin 43 gap junction modulators and connexin 43 hemichannel blockers or modulators (e.g. peptidomimetics and small molecules).

In some embodiments, doses of a connexin modulator, e.g. lufepirsen, are administered at Days 1, 2 and 14. In some embodiments, connexin modulator doses, e.g. lufepirsen, are administered on Days 1, 2, 14 and 28 (e.g. if the surface defect does not heal following administrations on Days 1, 2 and 14). In some embodiments, connexin modulator doses, e.g. lufepirsen, are administered on Days 1, 2, 7, 14, and 21 (e.g. if the surface defect does not heal following administrations on Days 1, 2, 14 and 28). In some embodiments, connexin modulator doses, e.g. lufepirsen, are administered on Days 1, 2, 7, 14, 21, and 28. In some embodiments, connexin modulator doses, e.g. lufepirsen, are administered on Days, 1, 2, 7, 14, 21, 28 and 35. In some embodiments, a dose is administered after Day 35. In some embodiments, therapeutically effective dose of a connexin modulator (e.g. lufepirsen) are administered on Days 1, 2 and 14, and on one more of Days 7, 21, 28 and 35. In some embodiments, administration using the doses and dose regimens described is to a PED or PCED. In some embodiments, the connexin modulator comprises a connexin antisense molecule (e.g. lufepirsen). In some embodiments, the connexin modulator comprises a connexin peptidomimetic (e.g., Peptide5, Gap19, XG19, Gap26, Gap27, aCT1, etc.). In some embodiments, the connexin modulator comprises a small molecule connexin hemichannel blocker (e.g., tonabersat). In some embodiments, the connexin modulator is a connexin 43 modulator.

In some embodiments, connexin modulator doses, e.g. lufepirsen, are administered on Days 1, 2, 14 and 21.

In some embodiments, connexin modulator doses, e.g. lufepirsen, are administered on Days 1, 2, 7, 14 and 21.

In some embodiments, connexin modulator doses, e.g. lufepirsen, are administered on Days 1, 2, 7, 14, 21 and 28.

In some embodiments, 3 doses of a connexin modulator (e.g. lufepirsen) are administered over 14 days to treat a non-healing or persistent ocular or corneal surface defect in a subject, including, for example, on Days 1 and 2. In some embodiments, 4 doses of a connexin modulator (e.g. lufepirsen) are administered over about 28 days. In some embodiments, 5 doses of a connexin modulator (e.g. lufepirsen) are administered over about to about 35 days. In some embodiments, 6-7 doses of a connexin modulator (e.g. lufepirsen) are administered over about 35 days. For example, in one embodiment, a connexin modulator (e.g. lufepirsen) is administered 4 times over Days 1 and 2, on about Day 14 and 28. In another embodiment, a connexin modulator (e.g. lufepirsen) is administered 5 times over Days 1 and 2, on about Day 14, on about Day 28 and on about Day 35. In another embodiment, a connexin modulator (e.g. lufepirsen) is administered 5 times over Days 1 and 2, on about Day 7, on about Day 14 and on about Day 28. In another embodiment, a connexin modulator (e.g. lufepirsen) is administered 6 times over Days 1 and 2, on about Day 7, on about Day 14, on about Day 21 and on about Day 28. In another embodiment, a connexin modulator (e.g. lufepirsen) is administered 7 times over Days 1 and 2, on about Day 7, on about Day 14, on about Day 21 and on about Day 28. Reference to “administration” here refers, of course, to administration of a connexin modulator in a single dose or in divided doses.

In some embodiments, the connexin modulator is a modulator of Cx26, Cx30, Cx31.1, Cx36, Cx37, Cx40, Cx43, Cx45, Cx50, Cx57 or any other connexin in the eye or blood vessels.

In some embodiments, the connexin modulator is a modulator of Cx26, Cx30, Cx30.3, Cx31, Cx31.1, Cx32, Cx43, Cx45, Cx50, and Cx58, or any other connexin in the corneal epithelium. Transcripts encoding these ten connexin isoforms have been detected by reverse transcriptase-polymerase chain reaction in both central and peripheral corneal epithelium. Six (Cx26, Cx31, Cx32, Cx43, Cx45, and Cx58) were confirmed by laser scanning confocal microscopy. Cx26 was detected throughout the central corneal epithelium and in the mid and superficial layers of the limbal epithelium. Cx43 and Cx45 were localized to the basal and suprabasal epithelial cells. Cx58 was expressed in the superficial epithelium throughout the cornea, and Cx31 and Cx32 were mainly expressed in the central corneal epithelium. See Yuan, X, et al., Expression Pattern of Connexins in the Corneal and Limbal Epithelium of a Primate. Cornea 28 (2): 194-199 (February 2009); Zhai, J, et al., Connexin expression patterns in diseased human corneas. Exp. Ther. Med. 7, 791-798 (2014). In some embodiments, the connexin modulator is a Cx43 connexin modulator, for example, a Cx43 expression modulator (e.g., an anti-Cx43 antisense), a Cx43 gap junction modulator or a Cx43 hemichannel modulator (e.g. an anti-connexin 43 peptidomimetic or a small molecule). In some embodiments, the modulator is a modified or unmodified antisense polynucleotide or peptidomimetic, e.g. a modified or unmodified Cx43 antisense polynucleotide or C43 peptidomimetic, or other corneal epithelial, endothelial or vascular connexin. In some embodiments, the modulator can include or exclude any of the foregoing connexins.

In some embodiments, the amount of connexin modulator administered to a subject with a non-healing ocular surface defect or disorder (e.g. a PED) or non-healing corneal surface defect or disorder (e.g. a PCED) in any of the dosing schedules described herein is a therapeutically effective amount. In some embodiments, a therapeutically effective amount of a connexin modulators is administered to the subject, for example, (1) on Days 1, 2 and 14, or (2) on Days 1, 2, 14 and, optionally, Day 28, or (3) on Days 1, 2, 7, 14, and about Day 21 and, optionally, on about Day 28 and 35, or (4) on the Days set forth in any of the other dosing schedules recited herein in addition to these dosing regimens (1)-(3). In some embodiments, a therapeutically effective amount of lufepirsen is administered.

In some embodiments, the amount of a connexin modulator administered to a subject with a non-healing ocular surface defect or disorder (e.g. a PED) or non-healing corneal surface defect or disorder (e.g. a PCED) in any of the dosing schedules described herein (e.g. (1) Days 1, 2, 14 and, optionally, Day 28 and/or Day 35; (2) Days 1, 2, 14 and optionally on Days 7, 21 and/or 28; (3) Days 1, 2, 14 and optionally on Days 7, 21, 28 and/or 35; (4) Days 1, 2 and 14 and any more or more or all of Days 7, 21 and 28, and optionally Day 35; or (5) on the Days set forth in other dosing schedules recited herein, etc., is one or more of the particular dose amounts described herein, including, for example, milligram per milliliter (mg/mL) connexin modulator dosing (e.g., a composition comprising or consisting essentially of about or at least about 0.6 mg/mL of a connexin modulator), micromolar (μM) connexin modulator dosing (e.g., a composition comprising or consisting essentially of a concentration of about or at least about 19 UM of a connexin modulator), milligram (mg) connexin modulator dosing (e.g., a composition comprising or consisting essentially of about or at least about 0.18 mg of a connexin modulator), and so on, etc.).

In some embodiments, the connexin modulator comprises an antisense molecule. See Examples 1 and 2, below. In some embodiments, the antisense molecule is a connexin43 antisense oligonucleotide. In some embodiments, the connexin modulator may be a connexin antisense polynucleotide comprising, consisting essentially of, or consisting of a sequence according to SEQ ID NOS.1-3, SEQ ID NOS: 4-16 and/or modified versions thereof. In some embodiments, the antisense oligonucleotide comprises, consists essentially of, or consists of 5′-GTA ATT GCG GCA AGA AGA ATT GTT TCT GTC-3′ (SEQ ID NO:1; lufepirsen). In some embodiments, the antisense oligonucleotide may be chemically modified or may be an unmodified oligonucleotide, e.g. a modified or an unmodified DNA oligonucleotide. In some embodiments, oligonucleotides are modified by changing onr or more of the phosphodiester linkages, the ribose backbone and/or the nucleobase. Other modifications are described herein. Other modifications are known in the art and described in various poublication, including, for example, in Shen, X. and Corey, DR, Chemistry, mechanism and clinical status of antisense oligonucleotides and duplex RNAs, Nuelic Acids Res. 2018 Feb. 28; 46 (4): 1584-1600. Methods of manufacture are also knowin the art. See, e.g., Abramova T, Frontiers and Approaches to Chemical Synthesis of Oligodeoxyribonucleotides, Molecules 2013 January; 18 (1): 1063-1075; Hao M, et al., Current and Emerging Methods for the Synthesis of Single-Stranded DNA Genes (Basel) 2020 Jan. 21; 11 (2): 116. See also Anwar, S., et al. Enhancing the Effectiveness of Oligonucleotide Therapeutics Using Cell-Penetrating Peptide Conjugation, Chemical Modification, and Carrier-Based Delivery Strate and Carrier-Based Delivery Strategies. Pharmaceutics 2023, 15, 1130.

In some methods of the invention, the connexin modulator administered to the non-healing surface defect or disorder in a subject is 0.06% lufepirsen. In some embodiments, the connexin modulator administered to a subject with a non-healing surface defect or disorder is 0.006% lufepirsen. In some embodiments, the connexin modulator administered is at least about 0.06% lufepirsen. In some embodiments, the connexin modulator administered to a subject with a non-healing surface defect or disorder is at least about 0.006% lufepirsen. In some embodiments, administration is to a PED or PCED. In some embodiments, a composition comprising or consisting essentially of a pharmaceutically acceptable carrier and about 0.06% lufepirsen, about 0.006% lufepirsen, at least about 0.06% lufepirsen or least about 0.006% lufepirsen is administered to heal the non-healing surface defect or disorder in a subject. Lufepirsen doses described herein and below may be prepared as described in Example 1. In some embodiments, the connexin modulator administered in the amounts described is a connexin antisense molecule other than (or in addition to) lufepirsen. In some embodiments, the connexin modulator administered in the amounts described is a connexin peptidomimetic (e.g. XG19). In some embodiments, the connexin modulator in the amounts described is a connexin hemichannel blocker (e.g. tonabersat).

In some embodiments, the non-healing surface defect or disorder is treated with composition comprising or consisting essentially of 0.6 mg/mL lufepirsen. In some embodiments of the present invention, the non-healing surface defect or disorder is treated with composition comprising or consisting essentially of 0.06 mg/mL lufepirsen. In some embodiments, the composition comprises or consists essentially of at least about 0.6 mg/mL lufepirsen. In some embodiments, the composition comprises or consists essentially of at least about 0.06 mg/mL lufepirsen. In some embodiments, administration is to a PED or PCED. In some embodiments, the connexin modulator administered in the amounts described is a connexin antisense molecule other than (or in addition to) lufepirsen. In some embodiments, the connexin modulator administered in the amounts described is a connexin peptidomimetic (e.g. XG19). In some embodiments, the connexin modulator in the amounts described is a connexin hemichannel blocker (e.g. tonabersat).

In some embodiments, the non-healing surface defect or disorder in a subject is treated with a composition comprising or consisting essentially of about 0.18 mg of lufepirsen in the regimens described herein. In some embodiments, each dose of lufepirsen applied to the non-healing surface defect or disorder in or on the subject comprises or consists essentially of at least about 0.18 mg of lufepirsen in the regimens described herein. In some embodiments of the present invention, the non-healing surface defect or disorder is treated with composition comprising or consisting essentially of about 0.018 mg of lufepirsen in the regimens described herein. In some embodiments, each dose of lufepirsen applied to the non-healing surface defect or disorder comprises or consists essentially of at least about 0.018 mg of lufepirsen in the regimens described herein. In some embodiments, administration is to a PED or PCED. In some embodiments, the connexin modulator administered in the amounts described is a connexin antisense molecule other than (or in addition to) lufepirsen. In some embodiments, the connexin modulator administered in the amounts described is a connexin peptidomimetic (e.g. XG19). In some embodiments, the connexin modulator in the amounts described is a connexin hemichannel blocker (e.g. tonabersat).

In some embodiments, the therapeutically effective amount of the modulator, e.g. connexin modulator, for example a connexin 43 modulator, connexin 43 gap junction modulator and/or connexin 43 hemichannel modulator, which is effective in methods of the invention, comprises a composition that is about, or at least about, 2 micromolar (2 μM), 5 micromolar (5 μM), 10 micromolar (10 μM), 15 micromolar (15 μM), or 20 micromolar (20 μM), including doses of about (or at least about) 1.9 micromolar (1.9 μM) or 1.9405 micromolar (1.9405 μM), and about (or at least about) 19 to 19.4 micromolar or 19.405 micromolar (19 to 19.4 μM or 19.405 μM), of a connexin modulator (e.g. lufepirsen), or any amount within or between any two of these recited dosages. Other effective doses that are effective in methods of the invention for the treatment of ocular surface defects or disorders (e.g. ocular PEDs and PCEDs) include about, or at least about, a composition comprising 30 micromolar (30 μM) or 40 micromolar (40 μM) of a connexin modulator, for example, a connexin 43 modulator, a connexin 43 gap junction modulator and/or a connexin 43 hemichannel modulator (e.g. lufepirsen). In some embodiments, the therapeutically effective amount of the connexin modulator administered as described is a connexin antisense molecule other than (or in addition to) lufepirsen. In some embodiments, the therapeutically effective amount of the connexin modulator administered as described is a connexin peptidomimetic (e.g. XG19). In some embodiments, the therapeutically effective amount of the connexin modulator as described is a connexin hemichannel blocker (e.g. tonabersat).

In some embodiments, about 0.18 mg (or other connexin antisense), about 0.018 mg of lufepirsen (or other connexin antisense or connexin modulator), or from about 0.1 mg, 0.2 mg, 0.3 mg, 0.4 mg or about 0.5 mg to about 1 mg of lufepirsen is administered to the non-healing surface defect or disorder in a single dose, or in two or more divided doses, e.g., for twice daily administration in up to 6 doses administered on Days 1, 2 and 14. In other embodiments, at least about 0.1 mg, 0.2 mg, 0.3 mg, 0.4 mg or 0.5 mg or at least about 1 mg of lufepirsen is administered in a single dose, or in two or more divided doses to the non-healing surface defect or disorder, e.g., for twice daily administration in up to 6 to 10 doses administered on Days 1, 2 and 14, or using other dose regimen described herein that includes Day 28 and/or Day 35. In other embodiments, at least about 0.5 mg or at least about 1 mg of lufepirsen is administered in single or divided doses, e.g., for twice daily administration, in up to 14 doses administered on Days 1, 2 and 14, and one or more of Day 7, Day 21, Day 28 and/or Day 35, for example. In some embodiments, administration is to a PED or PCED in a subject. In some embodiments, other connexin modulators-including e.g. other connexin 43 modulators (and/or modulators of other connexins), and other antisense compounds, peptidomimetics and/or small molecules)—are administered in these dose amounts on these days. In some embodiments, the connexin modulator administered in the amounts described is a connexin antisense molecule other than (or in addition to) lufepirsen. In some embodiments, the connexin modulator administered in the amounts described is a connexin peptidomimetic (e.g. XG19). In some embodiments, the connexin modulator in the amounts described is a connexin hemichannel blocker (e.g. tonabersat).

In any of the embodiments of these doses and dosing regimens and methods, the lufepirsen doses may be administrated to a PED or PCED in a subject. In some embodiments of these doses and dosing regimens and methods, the connexin modulator dose administered comprises another connexin antisense or modulator, e.g., another Cx43 antisense or Cx43 modulator other than or in addition to lufepirsen, or a modulator of another ocular or corneal epithelial connexin other than or in addition to Cx43.

In some embodiments of the present invention, the volume of a composition comprising or consisting essentially of a connexin modulator administered a non-healing surface defect or disorder in a subject is about 300 μL. In other embodiments, the volume of the composition administered is from about 250 μL to about 1.0 mL. In some embodiments, the composition comprises about 0.01 mg, 0.018 mg, 0.18 mg to about 0.2 mg of lufepirsen, or from about 0.18 mg or 0.20 mg or 0.50 mg to about 1 mg of lufepirsen. In some embodiments, the composition comprises at least about 0.018 mg lufepirsen, at least about 0.18 mg of lufepirsen, at least about 0.5 mg of lufepirsen or at least about 1.0 mg of lufepirsen. In some embodiments, other connexin modulators, including e.g. other connexin 43 modulators other than lufepirsen (and/or modulators of other connexins), and other antisense compounds, peptidomimetics and/or small molecules), are administered in these amounts.

In some embodiments, the modulator is a small molecule connexin antagonist. In some embodiments, the modulator is a small molecule connexin antagonist comprises a compound according to Formula I, which includes tonabersat. Tonabersat, a benzoylamino benzopyran, is a modulator of gap junction channel and hemichannel activity, including connexin 43. In particular, tonabersat can block or inhibit hemichannels comprising connexin 43.

In some embodiments, the modulator is a connexin peptidomimetic. Connexin modulators including Peptide5 inhibit Cx43 hemichannel activity and/or ATP release during and following injury. Other connexin modulator peptidomimetics include Gap19 and XG19, and analogues thereof.

The instant inventions provide, inter alia, (1) methods for treating a non-healing ocular surface or corneal defect or disorder in a subject (e.g. a PED or a PCED) by administration of a therapeutically effective amount of a connexin modulator (e.g., a connexin expression modulator), a connexin gap junction modulator and/or a connexin hemichannel modulator in a dose regimen described herein to close and heal the defect or disorder; (2) methods for treating a non-healing ocular surface or corneal defect or disorder in a subject (e.g. a PED or a PCED) by administration of the connexin modulator doses described herein (e.g. in any of the dosing regimens described herein; and (3) the use of such modulators, including, for example, antisense modulators, peptidomimetic modulators and small molecule modulators, in the manufacture of a medicament (e.g. a pharmaceutical composition), and kits containing said medicaments that include or are associated with instructions for use in a method of the invention. See, e.g., Example 2, which describes clinically confirmed pulse dosing regimens for treating and healing non-healing persistent ocular defects.

In some embodiments, instant inventions provide, inter alia, (1) methods for treating uveitis, blepharitis, Sjögren's syndrome-associated keratoconjunctivitis sicca, Fuchs' dystrophy, and all forms of dry eye disease (e.g., evaporative dry eye, aqueous-deficient dry eye, and decreased wettability dry eye) in a subject by administration of a therapeutically effective amount of a connexin modulator (e.g., a connexin expression modulator, a connexin gap junction modulator and/or a connexin hemichannel modulator in a dose regimen described herein to treat the disorder; (2) methods for treating uveitis, blepharitis, Sjögren's syndrome-associated keratoconjunctivitis sicca, Fuchs' dystrophy, and all forms of dry eye disease (e.g., evaporative dry eye, aqueous-deficient dry eye, and decreased wettability dry eye) in a subject by administration of the connexin modulator doses described herein (e.g. in any of the dosing regimens described herein; and (3) the use of such modulators, including, for example, antisense modulators, peptidomimetic modulators and small molecule modulators, in the manufacture of a medicament (e.g. a pharmaceutical composition), and kits containing said medicaments that include or are associated with instructions for use in a method of the invention for treating uveitis, blepharitis, Sjögren's syndrome-associated keratoconjunctivitis sicca, Fuchs' dystrophy, and dry eye disease (e.g., evaporative dry eye, aqueous-deficient dry eye, and decreased wettability dry eye).

All non-healing or persistent ocular surface or corneal (e.g. corneal epithelial) defect or disorder of any etiology may be treated using the compositions and methods of the invention, including the dose and dose regimens. In some embodiments, the non-healing ocular surface defect or disorder is a PED. In some embodiments, the non-healing ocular surface defect or disorder is a PCED. In some embodiments, the non-healing ocular surface defect or disorder is a corneal ulcer. In some embodiments, the non-healing ocular surface or corneal defect or disorder results from a chemical and/or thermal injury. Non-healing ocular surface or corneal defects or disorders can occur following exposure to toxic agents, chemicals, mechanical injury, thermal insult, and ocular surface or corneal infections. In some embodiments, the non-healing ocular surface or corneal defect or disorder results from inflammation, or from an inflammatory disease. In some embodiments, the non-healing ocular surface or corneal defect or disorder results from inflammation of the eye. In some embodiments, the non-healing ocular surface or corneal defect or disorder results from an inflammatory disease of the eye, the ocular surface or the cornea. In some embodiments, the non-healing ocular surface defect or disorder results from a physical trauma or injury, including mechanical injury. In certain embodiments, the non-healing ocular surface defect or disorder is a PED or PCED resulting from a chemical and/or thermal injury. In some embodiments, the chemical and/or thermal injury resulting in a PCED is a severe chemical and/or thermal injury. In some embodiments, an ocular surface and/or corneal burn or injury to be treated in a method of the invention has greater than 50% conjunctival or >6 clock hours of limbal involvement. In some embodiments, the ocular surface and corneal burns and injuries to be treated by a method of the invention is >3 on the Dua Classification scale. In some embodiments of the invention, severe ocular and corneal burns and injuries are particularly responsive to treatments with connexin 43 modulators (e.g. lufepirsen) and/or modulators of other connexins using the methods described herein All PED and PCED etiologies, no matter the cause, may be treated using the methods of the invention as described herein. PED and PCED etiologies include not only inflammation and inflammatory disease, but chemical, mechanical, thermal causes, neurotrophic keratitis and limbal stem cell deficiency. In some embodiments, methods and/or doses of the invention are used to treat PED and/or PCED caused by or resulting from chemical injury. In some embodiments, methods and/or doses of the invention are used to treat PED and/or PCED caused by or resulting from thermal injury. In some embodiments, methods and/or doses of the invention are used to treat PED and/or PCED caused by or resulting from mechanical injury. In some embodiments, methods and/or doses of the invention are used to treat PED and/or PCED caused by or resulting from inflammation. In some embodiments, methods and/or doses of the invention are used to treat PED and/or PCED caused by or resulting from neurotrophic keratitis.

In some embodiments, the non-healing ocular surface defect or disorder is a corneal ulcer. In some embodiments, the corneal ulcer is caused by a bacterium, a virus, a fungus or a parasite. In some embodiments, the corneal ulcer results from acanthamoeba keratitis, fungal keratitis or herpes simplex keratitis. In other embodiments, the non-healing ocular surface defect or disorder, e.g. a corneal ulcer, is caused by a trauma such as an abrasion (e.g. scratch or cut) or a foreign body in the eye. In another embodiment, the non-healing ocular surface defect or disorder, e.g. a corneal ulcer, is caused by an allergy or an allergic eye disease. In another embodiment, the non-healing ocular surface defect or disorder, e.g. a corneal ulcer, is caused by dry eye. In some embodiments, the non-healing ocular surface defect or disorder, is a burn. In some embodiments, the non-healing ocular surface defect or disorder is a corneal burn or an ocular or corneal burn ulcer.

In some embodiments, methods of the invention, the doses and the dose regimens, and the connexin modulators (e.g. connexin expression modulators, connexin gap junction modulators and connexin hemichannel modulators, including, for example, modulators of Cx26, Cx30, Cx30.3, Cx31.1, Cx32, Cx36, Cx37, Cx40, Cx43, Cx45, Cx50 or Cx58 connexins, gap junctions and/or hemichannels) described herein are used for the treatment of uveitis, i.e. inflammation of middle layer of the eye, the uvea, and surrounding tissue. In some embodiments, connexin 43 modulators are used to treat one or more forms of uveitis. In some embodiments, other connexin modulators are used, including, for example, connexin 26 modulators, connexin 45 modulators or others, alone or in combination.

In some embodiments the uveitis to be treated as described herein is anterior uveitis (which affects, e.g., the iris at the front of the eye).

In some embodiments the uveitis to be treated as described herein is intermediate uveitis (which affects, e.g., the ciliary body and the vitreous).

In some embodiments the uveitis to be treated as described herein is posterior uveitis (which affects, e.g., the retina and the choroid at the back of the eye).

In some embodiments the uveitis to be treated as described herein is panuveitis (which affects, e.g., all parts of the uvea, from the front to the back of the eye).

In some embodiments, the uveitis to be treated as described herein is caused, in whole in or part, by one or more of ocular trauma, ocular injury, ocular surgery, infection (including, for example, cytomegalovirus (CMV) retinitis, histoplamosis, reactive arthritis, shingles, syphilis, toxoplamosis, cat-scratch disease, herpes zoster, tuberculosis, etc.), inflammation, inflammatory disease and an autoimmune disorder (including, for example, AIDS, Behcet's disease, lupus, multiple sclerosis, psoriasis, rheumatoid arthritis, ulcerative colitis, Vogt-Koyanagi-Harada (VKH) disease, etc.). In some embodiments, the uveitis to be treated as described herein is caused, in whole in or part, by an autoimmune or inflammatory disorder that affects other parts of the body, such as sarcoidosis, systemic lupus erythematosus or Crohn's disease. In some embodiments, the uveitis to be treated as described herein is caused, in whole in or part, by ankylosing spondylitis. In some embodiments, the uveitis to be treated as described herein occurs for no known reason.

In these uveitis treatment embodiments, one or more symptoms of uveitis are alleviated, in whole or in part, using methods of the invention, the doses and the dose regimens, and the connexin modulators as described herein, which may be administered alone or together with (or co-formulated with) other agents for the treatment of uveitis (e.g. antibiotics, antivirals or antifungals, dilating eye drops; steroidal anti-inflammatoiremmunosuppressants. Symptoms of uveitis alleviated when treated as described herein include eye pain, eye redness, light sensitivity, blurred vision, dark/floating spots in the field of vision (floaters) and decreased vision.

In some embodiments, methods of the invention, the doses and the dose regimens, and the connexin modulators (e.g. connexin expression modulators, connexin gap junction modulators and connexin hemichannel modulators, including, for example, modulators of Cx26, Cx30, Cx30.3, Cx31.1, Cx32, Cx36, Cx37, Cx40, Cx43, Cx45, Cx50 or Cx58 connexins, gap junctions and/or hemichannels) described herein are used for the treatment of blepharitis. In some embodiments, the subject is treated for anterior blepharitis. In some embodiments, the subject is treated for posterior blepharitis. In some embodiments, one or more of the connexin modulators described or referenced herein administered with, or are formulated for administration or co-administration to the subject with, one more anti-microbial agents (e.g. erythromycin, bacitracin) and/or one or more immunomodulatory agents (e.g. a cyclosporine) and/or other blepharitis treatment agents for use in the methods and regimens described herein to treat blepharitis. In some embodiments, one or more of the connexin modulators described or referenced herein are administered with, or co-formulated with, one more anti-inflammatory agents (e.g. a steroid) for use in the methods and regimens described herein to treat blepharitis. In these blepharitis treatment embodiments, one or more symptoms of blepharitis are alleviated, in whole or in part, using methods of the invention and connexin modulators as described herein, including the doses and dose regimens. Symptoms of blepharitis alleviated when treated as described herein include feeling like there's something in the eye, burning or stinging eyes, watery eyes, itchy eyes, sensitivity to light, red and swollen eyes or eyelids, dry eye, crusty eyelids or eyelashes on waking, and blurry vision.

Dry eye is an ocular surface disease. In some embodiments, methods of the invention, the doses and the dose regimens, and the connexin modulators (e.g. connexin expression modulators, connexin gap junction modulators and connexin hemichannel modulators, including, for example, modulators of Cx26, Cx30, Cx30.3, Cx31.1, Cx32, Cx36, Cx37, Cx40, Cx43, Cx45, Cx50 or Cx58 connexins, gap junctions and/or hemichannels) described or referenced herein are used for the treatment of a subject with any stage of dry eye disease (DED), also known as dry eye syndrome (DES), keratoconjunctivitis sicca (KCS), and keratitis sicca. All stages of DED may be treated using the methods, doses, dose regimens, compositions and kits described herein, including DED stage 1, DED stage 2, DED stage 3 and DED stage 4. In some embodiments, the methods of the invention, the doses and the dose regimens, and compositions of the invention may be used alone or in conjuction with artificial tears and/or systemic or topical anti-inflammatoirer the treatment of DED. In some embodiments, the connexin modulator doses for use as described herein are formulated with or within an artificial tear composition or with an anti-inflammatory and may be used for the treatment of DED. In some embodiments, the connexin modulator doses for use as described herein are formulated with anti-inflammatory compounds or compositions for topical administration in the treatment of DED, using known effective doses of at least one inflammatory. In some embodiments, the connexin modulator doses for use as described herein are administered alone or in conjunction with one or more DED treatments (e.g. Tyrvaya, Rstasis, Cequa, Xiidra, Eysuvis). In some embodiments, the connexin modulator doses for use as described herein are formulated with one or more DED treatments for administration together (e.g., one more connexin modulators with one more other dry eye treatments, such as for example, Tyrvaya, Rstasis, Cequa, Xiidra, Eysuvis). In some embodiments, the connexin modulator doses for use as described herein are formulated with at least one corticotocosteroid for co-administation. In these dry eye treatment embodiments, one or more symptoms of dry are alleviated, in whole or in part, using methods of the invention and connexin modulators as described herein, including the doses and dose regimens. Symptoms of dry eye alleviated when treated as described herein include dry, gritty, stinging or burning sensations in the eyes, redness, watery or teary eyes, and mucus that make the eyes feel “glued shut” after sleeping. Many people with dry eye also report the feeling of something in the eye or eyestrain, blurry vision or eye fatigue. Itching and light sensitivity are other symptoms of dry eye.

In other embodiments, methods of the invention, the doses and the dose regimens, and the connexin modulators described or referenced herein are used for the treatment of a subject for Sjögren's syndrome-associated KCS and one or more symptoms of Sjögren's syndrome-associated KCS are treated or are alleviated, in whole or in part. Symptoms of Sjögren's syndrome-associated KCS alleviated when treated as described herein include include itching, burning, a gritty, pulling, or foreign body sensation and/o or photosensitivity. A sharp stabbing pain, eye strain or fatigue, and blurred vision may also occur. Some patients note a flood of tears after severe irritation.

In other embodiments, methods of the invention, the doses and the dose regimens, and the connexin modulators described or referenced herein are used for the treatment of a subject for Fuchs' dystrophy, and the treatment results in the alleviation of one or more symptoms of Fuchs' dystrophy, in whole or in part. One of the first signs and symptoms of Fuchs' dystrophy is blurred vision, caused by fluid buildup in the cornea. In early stages of the disease, the excess fluid builds up overnight during sleep, causing blurry vision and/or discomfort when waking in the morning that can last for hours. In later stages of Fuchs' dystrophy, the periods of swelling, impaired vision and discomfort last longer, even up to the entire day. Other symptoms of Fuchs' dystrophy include rough or gritty feelings in the eye, occasionally accompanied by sharp eye pains, discomfort in bright light, fluctuating eyesight throughout the day or day to day, halos and/or glares from bright lights, and blurry vision combined with poor contrast in colors.

In some embodiments, one or more of the connexin modulators described or referenced herein are formulated with one more anti-microbial agents for use in the methods and regimens described herein.

In some embodiments, one or more of the connexin modulators described or referenced herein are formulated with one more anti-bacterial agents for use in the methods and regimens described herein.

In some embodiments, one or more of the connexin modulators described or referenced herein are formulated with one more anti-parasitic agents for use in the methods and regimens described herein.

In some embodiments, one or more of the connexin modulators described or referenced herein are formulated with one more anti-fungal agents for use in the methods and regimens described herein.

In some embodiments, one or more of the connexin modulators described or referenced herein are formulated with one more anti-viral agents for use in the methods and regimens described herein.

In some embodiments, one or more of the connexin modulators described or referenced herein are formulated with one more compounds selected from the group consisting of anti-microbial agents, anti-bacterial agents, anti-parasitic agents, anti-viral agents, anti-fungal agents and lubricating agents.

### Definitions

It is understood that for various reasons, including the ability of patients to travel to a clinic or to an office or site for administration of a drug, patients' schedules, the availability of drug, etc., the day of administration of a connexin modulator pursuant to a dose regimen as described herein may need to be (and can be) adjusted. The term “about” when used with respect to the administration Day for a connexin modulator pursuant to an administration regimen described herein means, generally, that Day plus or minus one day. Thus, administration on “about Day 28” means administration on Day 28, but also includes administration on Day 27 or Day 29, for example.

As used herein, the term “connexin modulator” (also sometimes referred to as a connexin “inhibitor” or “antagonist”) is a compound that prevents, inhibits, and/or reduces the function or activity of a connexin, a connexin gap junction channel or a connexin hemichannel, alone, together or separately, including, for example, prevention, inhibition and/or reduction in the function and/or activity and/or the formation of connexin hemichannels and/or connexin gap junctions, including the expression of a connexin protein, its trafficking and/or assembly. Such functions and activities may include, for example, docking of hemichannels on adjacent cells and opening to form a gap junction channel. They may also include intercellular communication between cells, and the flow of molecules between cells through a gap junction channel. Modulation of a hemichannel is the modulation of one or more functions and/or activities of a hemichannel, for example, the flow of molecules through a hemichannel. Such functions and activities may include, for example, the flow of molecules from the extracellular space or environment through a hemichannel into a cell, and/or the flow of molecules (e.g. adenosine triphosphate (ATP)) through a hemichannel from the intracellular space or environment of a cell into the extracellular space or environment. Prevention, inhibition and/or reduction of function or activity may be direct or indirect (for example, but not limited to, directly blocking a channel, inducing a conformational change, or modifying a connexin phosphorylation state or opening probability). Modulation of a hemichannel also includes suppressing the permeability of a hemichannel (e.g. lessening the flow of permeants through a hemichannel). In some embodiments, the permeant is adenosine triphosphate (ATP). The connexin modulator may be of any chemical nature. However, by way of example, the connexin modulator may be a nucleic acid (including antisense molecules, RNAi molecules, morpholinos, and other nucleic acids as described herein), a peptide or peptidomimetic, or a small molecule or other chemical. In certain embodiments, a connexin modulator is a compound that targets one or more components of a gap junction, including connexins, hemichannels, to inhibit or block its activity, expression, trafficking and/or assembly. “Inhibits” or “blocks” or “antagonizes” or “modulates” should not be taken to imply that the function, activity, expression, trafficking and/or assembly of a connexin, a connexin hemichannel (e.g. a connexin 43 hemichannel) or gap junction is completely inhibited or blocked or wholly antagonized or modulated, although this may be preferred, but should be taken to include any reduction in the function, activity, expression, trafficking and/or assembly of a connexin (including its transcription, translation and/or expression), a connexin hemichannel (including its permeability or its opening, or release of ATP, to the extracellular environment) or gap junction (including its opening to an adjacent cell, or creation from hemichannels in adjacent cells). Connexin modulators as used herein include connexin expression modulators, connexin gap junction modulators and connexin hemichannel modulators.

In some embodiments, in addition to Cx43 modulators, the connexin modulator is a modulator of any other connexin in the corneal epithelium, including Cx26, Cx30, Cx30.3, Cx31, Cx31.1, Cx32, Cx45, Cx50, and Cx58. In some embodiments, the connexin modulator may be a modulator of a connexin present in blood vessels, for example, a Cx37 modulator, a Cx40 modulator, a Cx43 modulator and/or a Cx45 modulator. Thus, as used herein, the term “connexin modulator” refers generally to connexin modulators, but also specifically to connexin 43 modulators and modulators of other connexins referenced herein (including connexin 43 and other gap junction and hemichannel modulators), and modulators of other blood vessel, endothelial and epithelial (e.g. ocular and corneal epithelial) connexins, gap junctions and hemichannels, unless otherwise provided. In some embodiments, the connexin modulator is a connexin43 modulator, e.g., a modulator of a connexin 43 expression, a connexin 43 hemichannel modulator that inhibits or blocks hemichannel opening or a connexin peptidomimetic. In some embodiments, the gap junction modulator is or includes modulators of other connexins found on the ocular surface, including the cornea and corneal epithelium, and their hemichannels and gap junctions. In other embodiments, the connexin modulator is a modulator of any other connexin in the eye or blood vessels, including Cx36 and Cx57 in addition to Cx37, Cx40, Cx43 and Cx45 as well as previously noted Cx26, Cx30, Cx31.1 and Cx50, which are also found in the corneal epithelium. In some embodiments, the modulating agent can include or exclude any of the noted connexins, connexin gap junction and/or connexin hemichannels described herein. In some embodiments, an ocular surface defect or disorder, for example, a PED, a PCED and/or an ocular ulcer (including, e.g., an inflammatory ocular surface ulcer, or an ocular surface ulcer, disorder or defect characterized at least in part by inflammation) is treated with a connexin modulator (e.g. a connexin expression modulator, a connexin gap junction modulator and/or a connexin hemichannel modulator) to inhibit activation of one or more inflammasomes. In some embodiments, connexin modulators inhibit activation of an inflammatory cascade by an inflammasome. In some embodiments, activation and/or activity of the NLRP3 inflammasome is modulated (e.g. blocked or downregulated) by a connexin modulator. In some embodiments, activation of an inflammatory cascade by the NLRP3 inflammasome is modulated (e.g. blocked or downregulated) by a connexin modulator. Inflammasome modulation assists in closing a non-healing ocular surface or corneal defect or disorder, including those described herein, and in the treatment of other diseases, conditions and disorders described herein. In some embodiments, an ocular surface defect or disorder, for example, a PED, a PCED and/or an ocular ulcer is treated with an inflammasome modulator (e.g. a modulator of the NLRP3 inflammasome) that may not be a connexin modulator (e.g. a connexin 43 hemichannel modulator). Inflammasome modulators include those described, for example, in Leung and Lowery, The patent landscape of inflammasome modulators. Nature Reviews Drug Discovery 19, 158 (2020). See also Chauhan, D., et al., Therapeutic modulation of inflammasome pathways. Immunol Rev. 297 (1): 123-138 (September 2020). Inflammasome modulators include selective G-protein coupled receptor 40 (GPR40) agonists (e.g. fasiglifam, which inhibits inflammasome activation by blocking formation of apoptosis-associated speck-like protein (ASC) containing a caspase recruitment domain (CARD), an inflammasome component), and ethyl pyruvate, which significantly suppresses activation of the NLRP3 inflammasome. Other inflammasome modulators unrelated to connexins and connexin hemichannels are known in the art. Modulation of the function of a hemichannel and/or a gap junction channel may occur by any means. However, by way of example only, modulation may occur by one or more of: preventing, blocking, inhibiting or decreasing the formation of a gap junction through hemichannel docking; inducing or promoting closure of a hemichannel; preventing, blocking, inhibiting or decreasing hemichannel opening; preventing, blocking, inhibiting or decreasing hemichannel permeability; inducing or promoting loss of coupling between hemichannels; triggering, inducing or promoting cellular internalization of a hemichannel and/or gap junction. Just as use of the words such as “blocking”, “inhibiting”, “preventing”, “decreasing” and “antagonizing”, and the like, should not be taken to imply complete blocking, inhibition, prevention, or antagonism, “inducing” or “promoting” should not be taken to imply complete prevention, block, inhibition or decrease in the formation of a gap junction through hemichannel docking; inducing or promoting the comlete closure of a hemichannel; completely preventing, blocking, inhibiting or decreasing hemichannel opening; completely preventing, blocking, inhibiting or decreasing hemichannel permeability; completely inducing or promoting loss of coupling between hemichannels; or triggering, inducing or promoting the complete loss of coupling or the complete internalization of a connexin hemichannel and/or gap junction (or group of hemichannels and/or gap junctions) and should be taken to include the partial induction or promotion of these things. Connexins, hemichannels and gap junctions may be present in cells of any type. Accordingly, reference to a “connexin,” “hemichannel” or “gap junction” should be taken to include reference to a connexin, a hemichannel or gap junction present in any cell type, unless the context requires otherwise. In one embodiment, the hemichannel is an epithelial cell connexin, hemichannel or gap junction (e.g., an ocular or corneal epithelial cell connexin, hemichannel or gap junction). In one embodiment, the connexin, hemichannel or gap junction is a vascular connexin, hemichannel or gap junction. In one embodiment, the connexin, hemichannel or gap junction is a connexin, hemichannel or gap junction found in vascular endothelial cells and/or vascular smooth muscle cells. A “hemichannel blocker” is a compound that interferes with the passage of molecules through a connexin hemichannel. A hemichannel blocker can block or decrease hemichannel opening, block, decrease or suppress hemichannel opening, block or reduce the release of molecules through a hemichannel to an extracellular space, and/or block or reduce the entry of molecules through a hemichannel into an intracellular space. Hemichannel blockers include compounds that fully or partially block hemichannel leak or the passage of molecules through a hemichannel (e.g. to or from an extracellular space). Hemichannel blockers also include compounds that decrease the open probability of a hemichannel. Open probability is a measure of the percentage of time a channel remains open versus being closed (reviewed in Goldberg G S, et al., Selective permeability of gap junction channels Biochimica et Biophysica Acta 1662 (2004) 96-101). Examples of hemichannel blockers include peptides, small molecules, antibodies and antibody fragments. Hemichannel blockers include hemichannel modulators. Hemichannel blockers may interfere directly, or directly, with the passage of molecules through a connexin hemichannel, or with the permeability of a hemichannel.

In some embodiments, the connexin modulator may be a modulator of a connexin hemichannel present in blood vessels, for example, a connexin 43 hemichannel modulator and/or connexin 37 hemichannel modulator, a connexin 40 hemichannel modulator, a connexin hemichannel modulator or another blood vessel connexin hemichannel. In some embodiments, the connexin modulator may be a modulator of a connexin hemichannel present in the ocular epithelium, e.g., the corneal epithelium. In some embodiments, the connexin modulator is a modulator of a Cx26, Cx30, Cx30.3, Cx31, Cx31.1, Cx32, Cx43, Cx45, Cx50, and/or Cx58 hemichannel, or any other connexin hemichannel in the corneal epithelium. In other embodiments, the connexin modulator is a modulator of any other connexin hemichannel in the eye or blood vessels, including Cx36 and Cx57 hemichannels in addition to Cx37, Cx40, Cx43 and Cx45 hemichannels as well as previously noted Cx26, Cx30, Cx31.1 and Cx50 hemichannels, which are also found in the corneal epithelium.

A “bandage contact lens” is any contact lens that can be used to protect the ocular surface. Bandage contact lenses are also referred to as “therapeutic contact lenses” and “therapeutic bandage contact lenses.” They also help relieve pain and can assist in healing. The bandage contact lens will allow mechanical protection of the ocular surface, e.g. the cornea, from eyelid movement, and will also protect the surface of the eye, e.g., the corneal surface, from atmospheric exposure and further ocular insult and will limit dehydration. Bandage lenses include hydrogel lenses and silicon hydrogel lenses, and are well known in art. See Lim and Lim, Therapeutic Contact Lenses in the Treatment of Corneal and Ocular Surface Diseases—A Review Asia-Pacific Journal of Ophthalmology 9 (6): 524-532 (November-December 2020); Jacobs et al., CLEAR Medical use of contact lenses, Cont Lens Anterior Eye 2021 April; 44 (2): 289-329. In some embodiments, the therapeutic bandage lens is, for example, a soft contact lens, a gas permeable (scleral) lens or a 3D-printed bio-gel. Suitable FDA-approved soft bandage lenses include Acuvue Oasys with Hydraclear Plus (Johnson & Johnson Vision), Air Optix Night & Day Aqua (Alcon), Pure Vision (Bausch+Lomb) and UCL 55% (United Contact Lens) and Kontur (Kontur Kontact Lens).

A “small molecule” is defined herein to include compounds with a molecular weight below about 600 daltons and is generally an organic compound. Small molecule connexin modulators include the compounds of Formula I. A small molecule can be an active agent of a prodrug. Small molecules prodrugs include the compounds of Formula II, which are prodrugs of the small molecule hemichannel and connexin modulator, tonabersat.

As used herein, “treatment” (and grammatical variations thereof such as “treat” or “treating”) refers to clinical intervention in an attempt to alter the course of the individual, tissue or cell being treated, and can be performed either for prophylaxis or during the course of clinical pathology. Desirable effects of treatment include, but are not limited to, closing a non-healing ocular defect or disorder (e.g. a PED or PCED) and preventing occurrence or recurrence of a non-healing ocular surface or corneal defect or disorder, alleviation of signs or symptoms, diminishment of any direct or indirect pathological consequences of the a non-healing ocular surface or corneal defect or disorder, decreasing the rate of progression of a non-healing ocular surface or corneal defect or disorder, amelioration or palliation of the a non-healing ocular defect or disorder, and remission or improved prognosis. The term does not necessarily imply that a subject is treated until total recovery, but this is generally the case with the pulse-dose delivery methods of the invention for treatment of a non-healing ocular surface or corneal defect or disorder. Accordingly, “treatment” may also include maintaining or promoting a complete or partial state of remission of a non-healing ocular defect or disorder. Durable wound closure may be defined as 100% skin re-epithelialization without drainage or dressing requirements at two consecutive evaluations at least 2 weeks, but no greater than 5 months, apart following closure after the last administration of a connexin modulator as described herein (e.g. on about Day 14, on about Day 28, or on about Day 35). In some embodiments, durable wound closure refers to wound closure for one month (e.g. 4 weeks, or 28 days). In some embodiments, “treatment” refers to treatment and clinical interventions as described above and herein or treating uveitis, blepharitis, Sjögren's syndrome-associated keratoconjunctivitis sicca, Fuchs' dystrophy, and dry eye disease (including evaporative dry eye, aqueous-deficient dry eye, and decreased wettability dry eye).

The term “preventing” means preventing in whole or in part, or ameliorating or controlling.

As used herein, “effective amount” or “therapeutically effective amount” refers to an amount effective, at dosages and for periods of time as described in the dose regimens herein, to achieve a desired result in treatment of a non-healing ocular or corneal defect or disorder (e.g. PED, PCED, ocular ulcer, corneal ulcer, etc). For example, and not by way of limitation, a “therapeutically effective amount” can refer to an amount of a connexin modulator compound or composition, including but not limited to those disclosed herein, that is able to close a non-healing ocular defect or disorder when administered in a dose regimen of the invention. In other embodiments, the result of treatment with an “effective amount” is durable and the non-healing ocular defect or disorder will remain closed following treatment. The doses disclosed herein are therapeutically effective amounts; however, the methods are not limited to those doses or dose amounts and include the use of other therapeutically effective amounts. In some embodiments, “effective amount” or “therapeutically effective amount” refers to an amount effective, at dosages and for periods of time as described in the dose regimens herein, to achieve a desired result in treatment of uveitis, blepharitis, Sjögren's syndrome-associated keratoconjunctivitis sicca, Fuchs' dystrophy, and dry eye disease (including evaporative dry eye, aqueous-deficient dry eye, and decreased wettability dry eye).

As used herein, “prophylactically effective amount” refers to an amount effective, for example, at administration on about Day 28 (and/or optionally on about Day 35), to achieve or assure a desired result or a desired prophylactic result, e.g. durable wound closure with nonrecurrence of the non-healing ocular defect or disorder. In some embodiments, a prophylactically effective amount of connexin modulator is administered to a subject with a closed ocular surface disorder (e.g. a close PED or PCED). In some embodiments, an effective amount of a connexin modulator is administered prophylactically to a subject with a closed ocular surface disorder (e.g. a PED or PCED). In some embodiments, “prophylactically effective amount” refers to an amount effective to achieve a desired prophylactic result in treatment of uveitis, blepharitis, Sjögren's syndrome-associated keratoconjunctivitis sicca, Fuchs' dystrophy, and dry eye disease (including evaporative dry eye, aqueous-deficient dry eye, and decreased wettability dry eye), including during or after the treatment of uveitis, blepharitis, Sjögren's syndrome-associated keratoconjunctivitis sicca, Fuchs' dystrophy, and any form of dry eye disease using the methods described herein.

The terms “peptide,” “peptidomimetic” and “mimetic” include synthetic or genetically manufactured chemical compounds that may have the same or substantially the same structural and functional characteristics of protein regions which they mimic. By way of example, these may mimic the extracellular loops of opposing connexins involved in connexon-connexon docking and cell-cell channel formation, and/or the extracellular loops of hemichannel connexins, as well as the intracellular C-terminus of, for example, connexin 43 and others. As used herein, “peptidomimetics” (also known as peptide mimetics) which include peptide and peptide-based compounds, also include such non-peptide-based compounds such as peptide analogs. Peptidomimetics that are structurally similar to therapeutically useful peptides can be used to produce an equivalent or enhanced therapeutic or prophylactic effect. For example, the first extracellular loops of Cx37, Cx40 as well Cx43 are mimicked by peptides under the Gap26 code. Gap27 and Peptide5, on the other hand, mimic regions of the second extracellular loop. Gap27 targets Cx32, Cx40 and Cx43, while Peptide5 is used for Cx43 inhibition. Other peptidomimetics include JM2 (VFFK-GVKDRVKGRSD; SEQ ID NO:134), ASH3, CT9 (RPRDDLEI; SEQ ID NO:135) and CT9-TAT, CT10 (SRPRDDLEI; SEQ ID NO: 136), aCT (RQPKIWFPNRRKPWKK-RPRPDDLEI (SEQ ID NO:137) in which the inhibitor peptide includes the Cx43 C-terminal amino acids 374-382 (RPRPDDLEI; SEQ ID NO: 141) that encompass the ZO-1-binding sequence, which is in turn attached at its N-terminus to a 16-amino acid antennapedia internalization vector) mimic the C-terminal tail of Cx43 and Gap24 (GHGDPLHLEEVKC; SEQ ID NO:138) reproduces a sequence of the cytoplasmic loop of Cx32 while TAT-Gap24 (YGRKKRRQRRRGHGDPLHLEEVKC; SEQ ID NO: 139) also mimics a portion of the intracellular loop of Cx43. Both L2 and Gap19 also mimic the cytoplasmic loop but of Cx43. Peptides useful in the compositions and methods of the invention are described and refrenced herein and include connexin mimetic peptides directed to connexin extracellular loops and connexin intracellular regions, and peptides that mimic regions of a connexin C-terminal tail (e.g. a connexin 43 C-terminus). Peptides and peptidomimetics useful in the compositions and methods of the invention include those set forth in Tables II and III.

In some embodiments, peptides and peptidomimetics are modified. In some embodiments, peptides and peptidomimetics are unmodified. Peptidomimetics are molecules able to mimic natural peptides and proteins. Generally, peptidomimetics are structural or functional mimics (e.g., identical or similar) to a paradigm polypeptide (i.e., a polypeptide that has a biological or pharmacological function or activity), but peptidomimetics can also be modified, e.g. have one or more peptide linkages optionally replaced by a linkage selected from the group consisting of, for example, —CH₂NH—, —CH₂S—, —CH₂—CH₂—, —CH═CH— (cis and trans), —COCH₂—, —CH(OH)CH₂—, and —CH₂SO—. Peptidomimetics can also have been chemically modified to include unnatural amino acid substitutions, backbone amide bond modifications, rigid scaffolds, the addition of hydrophobic residues, and by other methods know in the art. The mimetic can be either entirely composed of natural amino acids, synthetic chemical compounds, non-natural analogues of amino acids, or, is a chimeric molecule of partly natural peptide amino acids and partly non-natural analogs of amino acids. The mimetic can also comprise any amount of natural amino acid conservative substitutions as long as such substitutions also do not substantially alter mimetic activity. In the case of connexins, these can mimic, for example, the extracellular loops of opposing connexins involved in connexon-connexon docking and cell-cell channel formation. For example, a mimetic composition can be useful as a gap junction modulating agent if it is capable of down-regulating biological actions or activities of hemichannels, such as, for example, preventing the docking of hemichannels to form gap-junction-mediated cell-cell communications, or preventing the opening of hemichannels to expose the cell cytoplasm to the extracellular millieu, etc. Peptidomimetics encompass those described herein, as well as those as may be known in the art, whether now known or later developed. Peptides and peptidomimetic connexin modulators may also be modified to increase stability, improve bioavailability and/or to increase cell membrane permeability.

The term “pharmaceutical formulation” refers to a preparation which is in such form as to permit the biological activity of an active ingredient contained therein to be effective, and which does not contain additional components that are unacceptably toxic to a subject to which the formulation would be administered. A “pharmaceutical composition” refers to a mixture of substances suitable for administering to a subject that includes an active ingredient or pharmaceutical agent. For example, a pharmaceutical composition may comprise a modified or unmodified antisense oligonucleotide and a sterile aqueous solution or a poloxamer or pluronic carrier.

A “pharmaceutically acceptable carrier,” as used herein, refers to an ingredient in a pharmaceutical formulation, other than an active ingredient, which can be safely administered to a subject. A pharmaceutically acceptable carrier includes, but is not limited to, a buffer, excipient, stabilizer, or preservative. Pharmaceutically acceptable carriers include poloxamers, e.g. Pluronic F-127. Pharmaceutically acceptable carriers for ocular administration will be ophthalmologically acceptable carriers suitable for application to (e.g. in or on) the eye.

As used herein, the term “subject” or the like, including “individual,” and “patient”, all of which may be used interchangeably herein, refers to any mammal, including humans, domestic and farm animals, and zoo, wild animal park, sports, or pet animals, such as dogs, horses, cats, sheep, pigs, cows, etc. The preferred mammal herein is a human, including adults, children, and the elderly. Preferred sports animals are horses and dogs. Preferred pet animals are dogs and cats. The subject may be, for example, an aquatic park animal, such as a dolphin, whale, seal or walrus. In certain embodiments, the subject, individual or patient is a human. Subjects are not meant to include animals used in scientific experiments (e.g. mice and rats).

As used herein, a “gap junction,” a “connexin gap junction” and a “connexin gap junction channel” refer to two hemichannels that connect across an intercellular space between adjacent cells and allow certain molecules to flow between those cells. They refer to the intercellular channels or clusters of intercellular channels that allow direct diffusion of ions and small molecules between adjacent cells. Gap junctions have been studies for decades and are well known in the art.

As used herein, the term “hemichannel” is a part of a gap junction (two hemichannels or connexons that connect across an intercellular space between adjacent cells to form a gap junction) and is comprised of a number of connexin proteins, typically homo-or hetero-meric hexamers of connexin proteins that form the pore for a gap junction between the cytoplasm of two adjacent cells. The hemichannel is supplied by a cell on one side of the junction, and by a cell on the other side, with two hemichannels from opposing cells normally coming together to form the complete intercellular gap junction channel. However, in some cells, and in cells under some circumstances, the hemichannel itself is active as a conduit between the cytoplasm and the extracellular space allowing the transference of ions and small molecules (e.g. ATP). Like their gap junction counterparts, connexin hemichannels have been long-studied and are also well known in the art.

Hemichannels and gap junction channels may be present in cells of any type. Reference to a “hemichannel” or a “gap junction channel” should be taken to include reference to a hemichannel or gap junction channel present in any ocular cell type, and in any ocular surface cell type, including cells of the cornea and/or corneal epithelium. In one embodiment of the invention, the hemichannel or gap junction channel is present in a cell in the eye. In one embodiment of the invention, the hemichannel or gap junction channel is present in a cell in the front of the eye, i.e., the anterior segment. In some embodiments of the invention, the hemichannel or gap junction channel is present in a cell in the cornea. In some embodiments of the invention, the hemichannel or gap junction channel is present in a cell in the corneal epithelium. In some embodiments of the invention, the hemichannel or gap junction channel is present in a cell in the microvasculature of the eye, including the corneal microvasculature. In some embodiments of the invention, the hemichannel or gap junction channel is present in a cell in the uvea. In some embodiments of the invention, the hemichannel or gap junction channel is present in a cell relating to dry eye disease, blepharitis (including e.g. cells of or relating to the eyelid margin), Sjögren's syndrome-associated keratoconjunctivitis sicca (including e.g. cells of or relating to the conjunctiva), Fuchs' dystrophy (including e.g. corneal endothelial cells).

A “non-healing ocular surface defect or disorder” means a ocular surface defect or disorder that does not completely heal or close within about ten days to two weeks of standard clinical care. Non-healing ocular surface defect or disorder include ocular ulcers, corneal ulcers, ocular persistent epithelial defects (PEDs) and persistent corneal epithelial defects (PCEDs). Ocular ulcers include corneal ulcers, and include those described herein, including infectious ulcers, chemical ulcers, burn ulcers, traumatic or trauma-induced ulcers, inflammatory ulcers, and mixed-cause ulcers. A “non-healing” or “persistent” ocular surface or corneal defect or disorder (including for example PEDs and PCEDs) results from the failure of re-epithelialization and closure within 10-14 days, even with standard supportive treatment.

The phrase “consisting essentially of” refers to the specified materials and those that do not materially affect the basic and novel characteristics of the medicament (or steps, in the case of a method). The basic and novel characteristics of the inventions are described throughout the specification, and include the ability of connexin modulators, connexin modulator compositions and methods of the invention to block or modulate connexins, connexin gap junctions and/or connexin hemichannels and to attenuate, block or inhibit, for example the production, function or activity of a connexin, a connexin gap junction and/or a connexin hemichannel, or inflammasome activity (e.g. hemichannel-mediated inflammasome activity, such as a connexin hemichannel-mediated inflammasome activity or pannexin hemichannel-mediated inflammasome activity). Material changes in the basic and novel characteristics of the inventions, including the medicaments and methods described herein, include an unwanted or clinically undesirable, detrimental, disadvantageous or adverse activity to healing a disease, disorder or condition described or referred to hearing, a slowing in healing, a diminution of connexin, connexin gap junction and/or connexin hemichannel modulation and/or attenuation, or an anwanted diminution of connexin, connexin gap junction and/or connexin hemichannel activity or function. In one embodiment, a composition or medicament of the invention will comprise, consist essentially of, or consist of a connexin 43 hemichannel blocker, for example, a connexin 43 antisense molecule, a connexin 43 peptidomimetic or small molecule connexin 43 hemichannel blocker or prodrug thereof.

### Antisense Modulators

A gap junction and/or connexin polynucleotide or oligonucleotide may be selected, for example, from modified or unmodified connexin polynucleotides or oligonucleotides, such as modified or unmodified connexin 43 antisense polynucleotides or oligonucleotides. In some embodiments, the modified connexin antisense polynucleotides, or oligonucleotides or polynucleotides comprise mixtures of modified and unmodified nucleotides. In some embodiments, the connexin 43 antisense compound used in the methods herein is an antisense oligonucleotide comprising naturally occurring nucleobases and an unmodified internucleoside linkage.

In some embodiments, the connexin 43 antisense compound is targeted to at least about 8 nucleobases of a nucleic acid molecule encoding a connexin having a nucleobase sequence selected from SEQ ID NO:17. The polynucleotides and oligonucleotides, for example, connexin 43 antisense compounds, may have from about 8 to about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, or about 80 nucleotides of SEQ ID NO:17, or a sequence complementary thereto, and/or the antisense polynucleotide or oligonucleotide may contain any range of lengths between any two of the recited lengths. The polynucleotides of this invention include synthesized polynucleotides having a length of less than 80 nucleotides, e.g., from 12-18 to about 50-80 nucleotides, preferably about 30 nucleotides or less, e.g., from 12 to about 30 nucleotides, and more preferably from about 15 to about 30 nucleotides. In one example, the polynucleotide has 30 nucleotides. The methods of this invention features, in some embodiments, the use of connexin 43 antisense compounds up to 40 nucleotides in length, for example, 15 to 40 nucleotides in length, comprising or consisting essentially of a nucleotide sequence selected from SEQ ID NO:1-17. The methods of this invention features, in some embodiments, the use of connexin 43 antisense compounds up to 40 nucleotides in length, for example, 15 to 40 nucleotides in length, comprising a nucleotide sequence selected from SEQ ID NO: 4-17.

| Human Cx 43, α1 | |
|---|---|
| LOCUS NM_000165 3088 bp mRNA linear PRI 26 Oct. 2004 | |
| DEFINITION Homo sapiens gap junction protein, alpha 1, 43 kDa | |
| (connexin 43) (GJA1), mRNA. | |
| (SEQ ID NO: 17) | |
| 1 | acaaaaaagc ttttacgagg tatcagcact tttctttcat tagggggaag gcgtgaggaa |
| 61 | agtaccaaac agcagcggag ttttaaactt taaatagaca ggtctgagtg cctgaacttg |
| 121 | ccttttcatt ttacttcatc ctccaaggag ttcaatcact tggcgtgact tcactacttt |
| 181 | taagcaaaag agtggtgccc aggcaacatg ggtgactgga gcgccttagg caaactcctt |
| 241 | gacaaggttc aagcctactc aactgctgga gggaaggtgt ggctgtcagt acttttcatt |
| 301 | ttccgaatcc tgctgctggg gacagcggtt gagtcagcct ggggagatga gcagtctgcc |
| 361 | tttcgttgta acactcagca acctggttgt gaaaatgtct gctatgacaa gtctttccca |
| 421 | atctctcatg tgcgcttctg ggtcctgcag atcatatttg tgtctgtacc cacactcttg |
| 481 | tacctggctc atgtgttcta tgtgatgcga aaggaagaga aactgaacaa gaaagaggaa |
| 541 | gaactcaagg ttgcccaaac tgatggtgtc aatgtggaca tgcacttgaa gcagattgag |
| 601 | ataaagaagt tcaagtacgg tattgaagag catggtaagg tgaaaatgcg aggggggttg |
| 661 | ctgcgaacct acatcatcag tatcctcttc aagtctatct ttgaggtggc cttcttgctg |
| 721 | atccagtggt acatctatgg attcagcttg agtgctgttt acacttgcaa aagagatccc |
| 781 | tgcccacatc aggtggactg tttcctctct cgccccacgg agaaaaccat cttcatcatc |
| 841 | ttcatgctgg tggtgtcctt ggtgtccctg gccttgaata tcattgaact cttctatgtt |
| 901 | ttcttcaagg gcgttaagga tcgggttaag ggaaagagcg acccttacca tgcgaccagt |
| 961 | ggtgcgctga gccctgccaa agactgtggg tctcaaaaat atgcttattt caatggctgc |
| 1021 | tcctcaccaa ccgctcccct ctcgcctatg tctcctcctg ggtacaagct ggttactggc |
| 1081 | gacagaaaca attcttcttg ccgcaattac aacaagcaag caagtgagca aaactgggct |
| 1141 | aattacagtg cagaacaaaa tcgaatgggg caggcgggaa gcaccatctc taactcccat |
| 1201 | gcacagcctt ttgatttccc cgatgataac cagaattcta aaaaactagc tgctggacat |
| 1261 | gaattacagc cactagccat tgtggaccag cgaccttcaa gcagagccag cagtcgtgcc |
| 1321 | agcagcagac ctcggcctga tgacctggag atctagatac aggcttgaaa gcatcaagat |
| 1381 | tccactcaat tgtggagaag aaaaaaggtg ctgtagaaag tgcaccaggt gttaattttg |
| 1441 | atccggtgga ggtggtactc aacagcctta ttcatgaggc ttagaaaaca caaagacatt |
| 1501 | agaataccta ggttcactgg gggtgtatgg ggtagatggg tggagaggga ggggataaga |
| 1561 | gaggtgcatg ttggtattta aagtagtgga ttcaaagaac ttagattata aataagagtt |
| 1621 | ccattaggtg atacatagat aagggctttt tctccccgca aacaccccta agaatggttc |
| 1681 | tgtgtatgtg aatgagcggg tggtaattgt ggctaaatat ttttgtttta ccaagaaact |
| 1741 | gaaataattc tggccaggaa taaatacttc ctgaacatct taggtctttt caacaagaaa |
| 1801 | aagacagagg attgtcctta agtccctgct aaaacattcc attgttaaaa tttgcacttt |
| 1861 | gaaggtaagc tttctaggcc tgaccctcca ggtgtcaatg gacttgtgct actatatttt |
| 1921 | tttattcttg gtatcagttt aaaattcaga caaggcccac agaataagat tttccatgca |
| 1981 | tttgcaaata cgtatattct ttttccatcc acttgcacaa tatcattacc atcacttttt |
| 2041 | catcattcct cagctactac tcacattcat ttaatggttt ctgtaaacat ttttaagaca |
| 2101 | gttgggatgt cacttaacat tttttttttt tgagctaaag tcagggaatc aagccatgct |
| 2161 | taatatttaa caatcactta tatgtgtgtc gaagagtttg ttttgtttgt catgtattgg |
| 2221 | tacaagcaga tacagtataa actcacaaac acagatttga aaataatgca catatggtgt |
| 2281 | tcaaatttga acctttctca tggatttttg tggtgtgggc caatatggtg tttacattat |
| 2341 | ataattcctg ctgtggcaag taaagcacac tttttttttc tcctaaaatg tttttccctg |
| 2401 | tgtatcctat tatggatact ggttttgtta attatgattc tttattttct ctcctttttt |
| 2461 | taggatatag cagtaatgct attactgaaa tgaatttcct ttttctgaaa tgtaatcatt |
| 2521 | gatgcttgaa tgatagaatt ttagtactgt aaacaggctt tagtcattaa tgtgagagac |
| 2581 | ttagaaaaaa tgcttagagt ggactattaa atgtgcctaa atgaattttg cagtaactgg |
| 2641 | tattcttggg ttttcctact taatacacag taattcagaa cttgtattct attatgagtt |
| 2701 | tagcagtctt ttggagtgac cagcaacttt gatgtttgca ctaagatttt atttggaatg |
| 2761 | caagagaggt tgaaagagga ttcagtagta cacatacaac taatttattt gaactatatg |
| 2821 | ttgaagacat ctaccagttt ctccaaatgc cttttttaaa actcatcaca gaagattggt |
| 2881 | gaaaatgctg agtatgacac ttttcttctt gcatgcatgt cagctacata aacagttttg |
| 2941 | tacaatgaaa attactaatt tgtttgacat tccatgttaa actacggtca tgttcagctt |
| 3001 | cattgcatgt aatgtagacc tagtccatca gatcatgtgt tctggagagt gttctttatt |
| 3061 | taaagtt ttaatttagt ataaacat |

Modified oligonucleotide may comprise, for example, one or more of the following selected components: a modified internucleoside linkage, for example, a phosphorothioate linkage, and a modified sugar moiety, for example, a conformationally-strained sugar, for example, a Linked Nucleic Acid (LNA) or Bridged Nucleic Acid (BNA).

The chemical modification of the antisense polynucleotides disclosed or referenced herein may enhance their resistance to nucleases and may enhance their ability to enter cells. For example, phosphorothioate oligonucleotides may be used. Other deoxynucleotide analogs include methylphosphonates, phosphoramidates, phosphorodithioates, N3′P5′-phosphoramidates and oligoribonucleotide phosphorothioates and their 2′-O-alkyl analogs and 2′-O-methylribonucleotide methylphosphonates. Alternatively mixed backbone oligonucleotides (“MBOs”) may be used. MBOs contain segments of phosphothioate oligodeoxynucleotides and appropriately placed segments of modified oligodeoxy- or oligoribonucleotides. MBOs have segments of phosphorothioate linkages and other segments of other modified oligonucleotides, such as methylphosphonate, which is non-ionic, and very resistant to nucleases or 2′-O-alkyloligoribonucleotides. Methods of preparing modified backbone and mixed backbone oligonucleotides are known in the art.

In some embodiments, an antisense polynucleotide disclosed or referenced herein may include an oligonucleotide sugar moiety that is a modified sugar moiety. In some embodiments, the modified sugar moiety can be a thiol-containing sugar moiety or a sugar moiety which is a conformationally-strained some embodiments, the conformationally-strained sugar can be a locked nucleotide (locked nucleic acid, or LNA). In some embodiments, the locked nucleotide can be selected from one of the following types: 2′-O—CH₂-4′ (oxy-LNA), 2′—CH₂—CH₂-4′ (methylene-LNA), 2′—NH—CH₂-4′ (amino-LNA), 2′-N(CH₃)—CH₂-4′ (methylamino-LNA), 2′-S—CH₂-4′ (thio-LNA), and 2′-Se—CH₂-4′ (seleno-LNA). In some embodiments, the conformationally-strained sugar can be a bridged nucleic acid (BNA). Some conformationally-strained sugar can be a locked nucleic acid are shown in Formula III and Formula IV in U.S. Pat. No. 10,465,188.

Also featured herein for use in methods of the invention are modified or unmodified epithelial, endothelieal, corneal epithelial and/or vascular endothelial connexin antisense polynucleotides comprising from 8 to about 80 nucleotides of an connexin extracellular loop, intracellular region, C-terminus or other region for modulating expression of one or more connexins selected from the group consisting of connexin 26, connexin 30, connexin 30.3, connexin 31, connexin 31.1, connexin 32, connexin 43, connexin 45, connexin and connexin 58. The polynucleotides include synthesized polynucleotides having a length of less than 80 nucleotides, e.g., from 12-18 to about 50-80 nucleotides, preferably about 30 nucleotides or less, e.g., from 12 to about 30 nucleotides, and more preferably from about 15 to about 30 nucleotides. In one example, the polynucleotide has 30 nucleotides.

In some embodiments of this invention, the connexin 43 or other antisense oligonucleotide or polynucleotide has at least about 80%, 85%, 90%, 95%, 97%, 98% or 99% homology to a polynucleotide having a sequence selected from SEQ ID NOs: 1 to 17. Connexin modulators that are oligonucleotides or polynucleotides may have at least about 80%, 85%, 90%, 95%, 97%, 98%, or 99% homology to an 8 to 80 nucleotide portion of their respective sequences.

TABLE I
List of some connexin 43 antisense oligonucleotide embodiments

| | SEQ ID | | ASN target |
|---|---|---|---|
| ASN code | NO. for ASN | | site |
| name | sequence | ASN sequence | from 5′ end |
| | SEQ ID NO: 1 | GTAATTGCGGCAAGAAGAAT | |
| | | TGTTTCTGTC | |
| | SEQ ID NO: 2 | GTAATTGCGGCAGGAGGAAT | |
| | | TGTTTCTGTC | |
| | SEQ ID NO: 3 | GGCAAGAGACACCAAAGAC | |
| | | ACTACCAGCAT | |
| 24501 | SEQ ID NO: 5 | ACCCATGTTGCCTGGGCACC | 237-256 |
| 30004 | SEQ ID NO: 6 | GTAGGCTTGAACCTTGTCAA | 281-300 |
| 37503 | SEQ ID NO: 7 | TCTCCCCAGGCTGACTCAAC | 372-389 |
| LP2/ | SEQ ID NO: 4 | CCAGGCTGACTCAACCGCTG | 368-385 |
| 37501 | | | |
| 47001 | SEQ ID NO: 8 | CAGAAGCGCACATGAGAGAT | 464-483 |
| 47002 | SEQ ID NO: 9 | GAAGCGCACATGAGAGATTG | 462-481 |
| 50501 | SEQ ID NO: 10 | AGTGTGGGTACAGACACAAA | 500-519 |
| LP3 | SEQ ID NO: 11 | CAGACACAAATATGATCTGC | 490-509 |
| 50506 | SEQ ID NO: 12 | ATATGATCTGCAGGACCCAG | 481-500 |
| 112304 | SEQ ID NO: 13 | GTAATTGCGGCAAGAAGAAT | 1134-1153 |
| 1233 | SEQ ID NO: 14 | AGGCTGTGCATGGGAGTTAG | 1233-1252 |
| 133704 | SEQ ID NO: 15 | CGCTGGTCCACAATGGCTAG | 1316-1335 |
| 133705 | SEQ ID NO: 16 | GCTGGCTCTGCTTGAAGGTC | 1335-1354 |

Table I lists the polynucleotide sequences of several embodiments of connexin 43 polynucleotide modulators useful in the methods of the invention. When sequences such as SEQ ID NO:1-16 are noted, they and other Cx43 and other connexin antisense compounds represent both modified and unmodified oligonucleotides or polynucleotides. In some embodiments, the linkages between the nucleotides, and the structure of the sugar moiety of the nucleotides may be modified. In some embodiments, the internucleoside linkage between any two nucleotides can be a standard phosphodiester linkage. In some embodiments, the internucleoside linkage between any two nucleotides can be a phosphorothioate linkage. For example, SEQ ID NO:1 can be one of the following selected structures: GₛTₛAₛAₛTTGCGGCAAGAAGAATTGTTTCₛTₛGₛTₛC (SEQ ID NO: 356), wherein s denotes a phosphorothioate linkage between the two nucleotides. As another non-limiting example, SEQ ID NO:1 can be (G)(T)(A)(A)TTGCGGCAAGAAGAATTGTTTC(T)(G)(T)(C)(SEQ ID NO: 357), wherein the parenthetical nucleotides have modified sugar moieties, as described below. In some embodiments the Cx43 antisense compounds may be modified by substituting one or more uridine nucleotides residues for one or more thymine nucleotides in SEQ ID NOs: 1-17 or in the sequence of another connexin (e.g., Cx26, C32, Cx45, etc.).

Certain connexin modulators, including for example connexin 43 modulators, provide downregulation of connexin expression (for example, by downregulation of mRNA transcription or translation) or otherwise decrease or inhibit the activity of the connexin protein, connexin hemichannels or gap junctions. In the case of downregulation, this will have the effect of reducing direct cell-cell communication by gap junctions, or exposure of cell cytoplasm to the extracellular space by hemichannels, at the site at which connexin expression is downregulated.

In certain embodiments, an anti-connexin antisense prevents, decreases or alters the activity or function of a hemichannel or a gap junction. As described herein, modulation of the gap junction activity or function by anti-connexin antisense compounds can lead to the closing of gap junctions, closing of hemichannels, and/or passage of molecules or ions through gap junctions and/or hemichannels.

Connexin modulators may also comprise one or polynucleotides selected, for example, from the group consisting of morpholino oligonucleotides, RNAi molecules, siRNA molecules, PNA molecules, DNAzymes, and 5′-end-mutated U1 small nuclear RNAs, and analogs of the preceding. These and other compounds may be used alone or in combination with one more connexi modulators.

Synthesis of antisense polynucleotides and other anti-connexin polynucleotides such as RNAi, siRNA, and ribozyme polynucleotides as well as polynucleotides having modified and mixed backbones can be performed. See e.g. Stein C. A. and Krieg A. M. (eds), Applied Antisense Oligonucleotide Technology, 1998 (Wiley-Liss).

The antisense polynucleotide may inhibit transcription and/or translation of a connexin protein (e.g. connexin 43). The antisense polynucleotide is generally antisense to connexin protein mRNA, for example, connexin 43. Such a polynucleotide may be capable of hybridizing to connexin protein mRNA and may thus inhibit the expression of connexin by interfering with one or more embodiments of connexin protein mRNA metabolism including transcription, mRNA processing, mRNA transport from the nucleus, translation or mRNA degradation. The antisense polynucleotide typically hybridizes to the connexin mRNA to form a duplex which can cause direct inhibition of translation and/or destabilization of the mRNA. Such a duplex may be susceptible to degradation by nucleases. Preferably the polynucleotide is a specific inhibitor of transcription and/or translation from the connexin 43 gene or mRNA, and does not inhibit transcription and/or translation from other genes or mRNAs.

The connexin modulator product may bind to the connexin 43 gene or mRNA either (i) 5′ to the coding sequence, and/or (ii) to the coding sequence, and/or (iii) 3′ to the coding sequence. The antisense polynucleotide may hybridize to part of the connexin protein mRNA, such as connexin 43 mRNA. Typically, the antisense polynucleotide hybridizes to the ribosome binding region or the coding region of the connexin protein mRNA. The polynucleotide may be complementary to a region of the connexin mRNA. For example, the polynucleotide may be the exact complement of a part of connexin mRNA. However, absolute complementarity is not required and polynucleotides which have sufficient complementarity to form a duplex having a melting temperature of greater than about 20° C., 30° C. or 40° C. under physiological conditions are particularly suitable for use in the present invention. Thus, the polynucleotide is typically a homologue of a sequence complementary to the mRNA. The polynucleotide may be a polynucleotide which hybridizes to the connexin protein mRNA under conditions of medium to high stringency such as 0.03M sodium chloride and 0.03M sodium citrate at from about 50° C. to about 60° C.

Antisense polynucleotides may be part of compositions which may comprise polynucleotides to more than one connexin protein. Preferably, one connexin protein to which polynucleotides are directed is connexin 43. Others include connexins found in the ocular and/or corneal epithelium. Some embodiments of the invention are described with reference to oligodeoxynucleotides. However, other suitable polynucleotides (such as RNA polynucleotides) may be used.

### Peptidomimetic Modulators

In some embodiments, connexin modulators, including connexin, connexin gap junction and connexin hemichannel modulators, useful in methods of the invention include not only connexin oligonucleotides or polynucleotides, such as connexin 43 antisense oligonucleotides or polynucleotides, and others, but also connexin peptides or peptidomimetics, such as connexin 43 peptides or peptidomimetics, sometimes referred to anti-connexin peptides or peptidomimetics, e.g., anti-connexin hemichannel blocking peptides or peptidomimetics, which may be used in methods of the invention and dosed according to the one or more of the schedules described herein. They include, for example, peptides or peptidomimetic connexin modulators comprising or consisting essentially of sequences corresponding to portions of connexin extracellular domains, connexin transmembrane regions, and connexin carboxy-terminus regions. In some embodiments, connexin, connexin gap junction and connexin hemichannel modulators useful in methods of the invention include connexin 43 peptides or peptidomimetics with sequences according to a portion of the amino acid sequence of SEQ ID NO: 100 (connexin 43 protein).

In some embodiments, the connexin modulators useful in methods of the invention for treating non-healing ocular defects or disorders (e.g. PED) in a subject, including non-healing corneal defects or disorders (e.g., PCED) are anti-connexin 43 peptides or peptidomimetics. In some embodiments, anti-connexin 43 peptides or peptidomimetic that may be used in methods of the invention and dosed according to the one or more of the schedules described herein is a peptide comprising or consisting essentially of a portion of an extracellular domain of a connexin (e.g. connexin 43) and/or a peptide comprising or consisting essentially of a portion of a carboxy-terminal portion of a connexin (e.g. connexin 43), including those described and/or referenced herein.

The protein sequence of connexin 43 is shown below.

```
Connexin 43
(SEQ ID NO: 100)
Met Gly Asp Trp Ser Ala Leu Gly Lys Leu Leu Asp Lys Val Gln Ala
1 5 10 15
Tyr Ser Thr Ala Gly Gly Lys Val Trp Leu Ser Val Leu Phe Ile Phe
                        20 25 30
Arg Ile Leu Leu Leu Gly Thr Ala Val Glu Ser Ala Trp Gly Asp Glu
                35 40 45
Gln Ser Ala Phe Arg Cys Asn Thr Gln Gln Pro Gly Cys Glu Asn Val
        50 55 60
Cys Tyr Asp Lys Ser Phe Pro Ile Ser His Val Arg Phe Trp Val Leu
65 70 75 80
Gln Ile Ile Phe Val Ser Val Pro Thr Leu Leu Tyr Leu Ala His Val
                                85 90 95
Phe Tyr Val Met Arg Lys Glu Glu Lys Leu Asn Lys Lys Glu Glu Glu
                        100 105 110
Leu Lys Val Ala Gln Thr Asp Gly Val Asn Val Asp Met His Leu Lys
                115 120 125
Gln Ile Glu Ile Lys Lys Phe Lys Tyr Gly Ile Glu Glu His Gly Lys
        130 135 140
Val Lys Met Arg Gly Gly Leu Leu Arg Thr Tyr Ile Ile Ser Ile Leu
145 150 155 160
Phe Lys Ser Ile Phe Glu Val Ala Phe Leu Leu Ile Gln Trp Tyr Ile
                                165 170 175
Tyr Gly Phe Ser Leu Ser Ala Val Tyr Thr Cys Lys Arg Asp Pro Cys
                        180 185 190
Pro His Gln Val Asp Cys Phe Leu Ser Arg Pro Thr Glu Lys Thr Ile
                195 200 205
Phe Ile Ile Phe Met Leu Val Val Ser Leu Val Ser Leu Ala Leu Asn
        210 215 220
Ile Ile Glu Leu Phe Tyr Val Phe Phe Lys Gly Val Lys Asp Arg Val
225 230 235 240
Lys Gly Lys Ser Asp Pro Tyr His Ala Thr Ser Gly Ala Leu Ser Pro
                                245 250 255
Ala Lys Asp Cys Gly Ser Gln Lys Tyr Ala Tyr Phe Asn Gly Cys Ser
                        260 265 270
Ser Pro Thr Ala Pro Leu Ser Pro Met Ser Pro Pro Gly Tyr Lys Leu
                275 280 285
Val Thr Gly Asp Arg Asn Asn Ser Ser Cys Arg Asn Tyr Asn Lys Gln
        290 295 300
Ala Ser Glu Gln Asn Trp Ala Asn Tyr Ser Ala Glu Gln Asn Arg Met
305 310 315 320
Gly Gln Ala Gly Ser Thr Ile Ser Asn Ser His Ala Gln Pro Phe Asp
                                325 330 335
Phe Pro Asp Asp Asn Gln Asn Ser Lys Lys Leu Ala Ala Gly His Glu
                        340 345 350
Leu Gln Pro Leu Ala Ile Val Asp Gln Arg Pro Ser Ser Arg Ala Ser
                355 360 365
Ser Arg Ala Ser Ser Arg Pro Arg Pro Asp Asp Leu Glu Ile
        370 375 380
```

In some embodiments, connexin 43 (Cx43) and other connexin peptide modulators useful in carrying out methods of the invention and dosed according to the one or more of the schedules described herein include peptides such as Peptide5, i.e., VDCFLSRPTEKT (SEQ ID NO: 107), as Gap19, i.e., KQIEIKKFK (SEQ ID NO:108), Gap26, i.e., VCYDKSFPISHVR (SEQ ID NO:102), Gap27, a peptide called alpha connexin carboxyl terminus 1 (αCT1), and more, each targeting different binding sites with varying specificity and size. The connexin mimetic peptide Gap27, targeted to the SRPTEKTIFII sequence (SEQ ID NO: 104)(amino acids 204-214) on the second extracellular loop of Cx43 (“LLIQWYIYGFSLSAVYTCKRDPCPHQVDCFLSRPTEKTIFII”, disclosed as SEQ ID NO: 109), is a versatile inhibitor of connexin-mediated communication. In some embodiments the connexin 43 modulator may comprise or consist essentially of, for example, a peptide or peptidomimetic comprising or consisting essentially of SRPTEKTIF (SEQ ID NO:110).

In addition to Peptide5, Gap19, Gap 26, and Gap 27, another peptidomimetic particularly useful in the compositions, doses and dosing methods and schedules, kits and articles of manufacture disclosed herein is the fusion peptide designated XG19, i.e., lclrpvGGKQIEIKKFK, wherein lower case denotes the D-isomer [SEQ ID NO:111]). The XG19 peptidomimetic and its connexin modulation actions are described in U.S. Pat. No. 11,466,069, incorporated in its entirety herein by reference, as noted above.

In some embodiments, the invention provides compositions for use in the methods of the invention for treating a disease or disorder of the eye comprising a construct comprising (a) a targeting carrier peptide derived from the X-protein of the Hepatitis B virus and (b) a peptide capable of interacting with an intracellular domain of a connexin. In some embodiments, the targeting carrier peptide derived from the X-protein of the Hepatitis B virus comprises an amino acid sequence selected from the group consisting of all targeting carrier peptide described in U.S. Pat. No. 11,466,069. In some embodiments, the peptide capable of interacting with an intracellular domain interacts with the intracellular domain of one or more of connexin Cx26, Cx30, Cx30.3, Cx31.1, Cx32, Cx36, Cx37, Cx40, Cx43, Cx45, Cx50 and Cx58, including those described herein. In some embodiments, the peptide capable of interacting with an intracellular domain interacts with the intracellular domain of connexin 43. In some embodiments, the peptide capable of interacting with the intracellular domain of connexin 43 includes any of the connexin 43 intracellular interacting peptides described in U.S. Pat. No. 11,466,069, as well as those peptides described herein that interact with the intracellular domain of connexin 43.

In some embodiments, the connexin 43 modulators which may be used in methods of the invention and dosed according to the one or more of the schedules described herein can comprise peptides having a sequence comprising or consisting essentially of, for example, one or more of the following sequences: “Peptide 1” ADCFLSRPTEKT (SEQ ID NO: 112), “Peptide 2” VACFLSRPTEKT (SEQ ID NO: 113), “Peptide 11” VDCFLSRPTAKT (SEQ ID NO: 114), “Peptide 12” VDCFLSRPTEAT (SEQ ID NO: 115), “Peptide 5” VDCFLSRPTEKT (SEQ ID NO: 107), “Mod1” CFLSRPTEKT (SEQ ID NO: 116) and “Mod2” LSRPTEKT (SEQ ID NO: 117). In some embodiments, the carboxy-terminus of an anti-connexin peptide or peptidomimetic modulator can be modified. In some embodiments, the carboxy-terminus modification can comprise n-alkyl chains which can optionally be further linked to hydrogen or other moieties. In some embodiments, the connexin 43 peptides can include or exclude any of the peptides listed above or disclosed herein.

In some embodiments, the peptide or peptidomimetic comprises or consists essentially of from 7 to 40 amino acids of a connexin including, e.g., SEQ ID NO: 101 (SRPTEKT), SEQ ID NO: 107 (VDCFLSRPTEKT) and does not comprise a connexin C-terminal peptide.

Anti-connexin agents include peptides having an amino acid sequence that comprises about 5 to 20 contiguous amino acids of a connexin protein such as connexin 43 (SEQ ID NO:100), peptides having an amino acid sequence that comprises about 8 to 15 contiguous amino acids of connexin 43, or peptides having an amino acid sequence that comprises about 11 to 13 contiguous amino acids of connexin 43. Other anti-connexin agents include a peptide having an amino acid sequence that comprises at least about 5, at least about 6, at least about 7, at least about 8, at least about 9, at least about 10, at least about 11, at least about 12, at least about 13, at least about 14, at least about 15, at least about 20, at least about 25, or at least about 30 contiguous amino acids of connexin 43. Other anti-connexin 43 modulators comprise the extracellular domains of connexin 43, for example, peptide or peptidomimetic comprising or consisting essentially of SRPTEKT (SEQ ID NO: 101) or VDCFLSRPTEKT (SEQ ID NO: 107).

In other anti-connexin compounds, mimetic peptides are based on the extracellular domains of connexin 43 corresponding to the amino acids at positions 37-76 and 178-208 of connexin 43 protein sequence. Thus, certain anti-connexin peptides useful in methods of the invention have an amino acid sequence comprising or consisting essentially of about 7 to about of the amino acids corresponding to the regions at positions 37-76 and 178-208 of the connexin 43 protein sequence. The peptides need not have an amino acid sequence identical to those portions of the connexin 43 protein sequence, and conservative amino acid changes may be made such that the peptides retain binding activity or functional activity in the assays described herein and otherwise known in the art. In other embodiments, mimetic peptides are based on peptide target regions within the connexin protein other than the extracellular domains (e.g. portions of the connexin 43 protein sequence not corresponding to positions 37-76 and 178-208).

In addition to therapeutically effective modified or unmodified peptide or peptidomimetics comprising or consisting essentially of a portion of an extracellular or transmembrane domain or C-terminal domain of a connexin 43, for example, other embodiments include modified or unmodified peptides or peptidomimetics comprising or consisting essentially of a portion of, for example, an extracellular or transmembrane domain of one or more other connexins found in the ocular and/or corneal epithelium may be used in methods of the invention and dosed according to the one or more of the schedules described herein. Other embodiments include modified or unmodified peptides or peptidomimetics comprising or consisting essentially of, for example, a portion of an extracellular or transmembrane domain of one or more other connexins found in blood vessels (e.g. endothelium, etc.), including ocular and/or corneal blood vessels.

In some embodiments, the anti-connexin peptidomimetic useful in a method of the invention and which may be dosed according to the one or more of the schedules described herein is a connexin 45 peptidomimetic modulator comprising portions of the connexin 45 protein that antagonize or inhibit or block connexin-connexin interactions. In some embodiments the connexin 45 modulator may comprise, for example, a peptide or peptidomimetic comprising or consisting essentially of a portion of the E2 or C terminal domain of connexin 45, for example, comprising SRPTEKT (SEQ ID NO: 101). The peptide or peptidomimetic may also comprise, for example DCFISRPTEKT (SEQ ID NO: 118). Exemplary peptide sequences for connexin 45 peptides and peptidomimetic modulators useful in methods of the invention are also provided in Table 63 of U.S. Pat. No. 10,465,188.

In some embodiments, a connexin modulator comprises a peptide comprising an amino acid sequence corresponding to a portion of a transmembrane region of connexin 45 or a C-terminal region of connexin 45. In particular non-limiting embodiments, for example, the anti-connexin compound is a peptide having an amino acid sequence that comprises about 3 to about 30 contiguous amino acids of the known connexin 45 sequence, a peptide having an amino acid sequence that comprises about 5 to about 20 contiguous amino acids of the known connexin 45 sequence, a peptide having an amino acid sequence that comprises about 8 to about 15 contiguous amino acids of the known connexin 45 sequence, or a peptide having an amino acid sequence that comprises about 11, 12, or 13 contiguous amino acids of the known connexin 45 sequence. Other non-limiting embodiments include an anti-connexin compound that is a peptide having an amino acid sequence that comprises at least about 3, 4, 5, 6, 7, 8, 9, 10, 11, 12, 13, 14, 15, 20, 25, or 30 contiguous amino acids of the known connexin 45 sequence. In certain anti-connexin compounds provided herein, mimetic peptides are based on the extracellular domains of connexin 45 corresponding to the amino acids at positions 46-75 and 199-228 of the known connexin 45 sequence. Thus, certain peptide described herein have an amino acid sequence corresponding to the regions at positions 46-75 and 199-228 of the known connexin 45 sequence. The peptides need not have an amino acid sequence identical to those portions of the known connexin 45 sequence, and conservative amino acid changes may be made such that the peptides retain binding activity or functional activity in the assays described herein and otherwise known in the art. In other embodiments, mimetic peptides are based on peptide target regions within the connexin protein other than the extracellular domains (e.g. portions of the known connexin 45 sequence not corresponding to positions 46-75 and 199-228). See WO2006/134494, disclosing various connexin sequences.

Some peptidomimetic connexin modulators useful in methods of the invention include VDCFLSRPTEKT (SEQ ID NO: 107) and SRPTEKTIFII (SEQ ID NO: 104), which bind, for example, to the Cx43 extracellular domain.

In some embodiments, the connexin 26 peptidomimetic connexin modulator useful in a method of the invention and dosed according to the one or more of the schedules described herein is Gap26. In other embodiments, the anti-connexin peptidomimetic for use in a method of the invention is a connexin32 peptidomimetic (e.g. INCTLQPGCNSV (SEQ ID NO: 103) or 32Gap27, i.e., SRPTEKTIFII (SEQ ID NO: 104) or a connexin50 peptidomimetic (e.g. TAT-Cx50L2, i.e., GGERAPLAADQGSVKKSSSSSKGTKK (SEQ ID NO:105) or TAT-Cx50CT, i.e., SRARSDDLTV (SEQ ID NO:106)).

In some embodiments, peptides and peptidomimetics include peptides and peptidomimetics useful for the inhibition of gap junction channels and hemichannels corresponding to specific sequences within extracellular loops E1 and E2 involving the conserved QPG and SHVR (SEQ ID NO: 359) motifs of E1 (Gap26 peptide) and the SRPTEK motif (SEQ ID NO: 360) in E2 (Gap27 peptide) as well as the cytoplasmic loop (Gap19 peptide). Useful peptidomimetic connexin modulators are described in U.S. Pat. No. 9,248,141 (“Methods of treatment by administering anti-connexin peptides and mimetics”). Other useful peptide connexin modulators are described in U.S. Pat. No. 11,466,069 (“Methods of treatment and novel constructs”), including XG19 and other constructs comprising a targeting carrier peptide derived from the X-protein of the Hepatitis B virus and a peptide capable of interacting with an intracellular domain of a connexin (e.g. connexin 43).

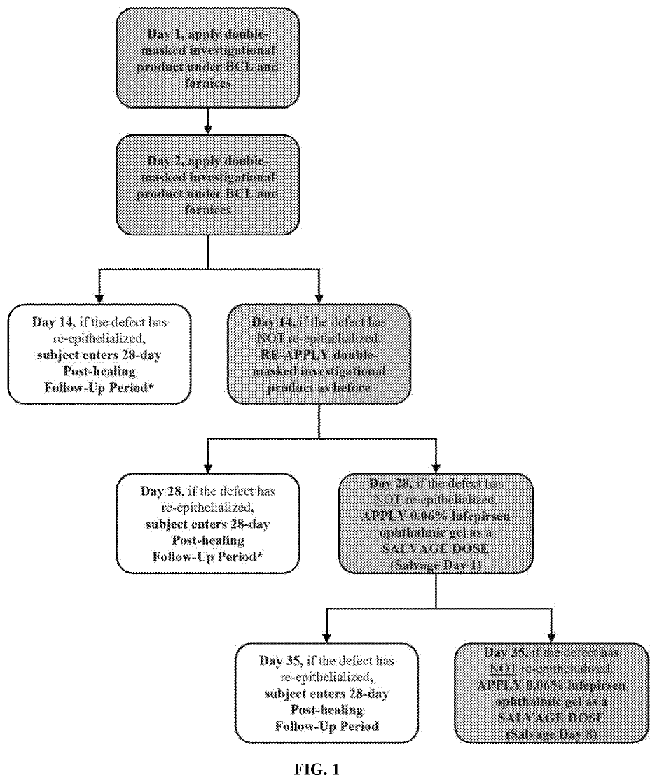

Other connexin peptide modulators useful in methods of the invention are provided in Table 64 of U.S. Pat. No. 10,465,188. Useful peptide modulators of connexin 43 (Cx43) and other connexins that may be dosed in embodiments of the invention are also referenced in Caufirez et al., and in King, D R, et al., Mechanisms of Connexin Regulating Peptides Int. J. Mol. Sci. 22:10186 (September 2021) and FIG. 1 (“Schematic of the Cx43 protein in the plasma membrane with colored lines indicating the positions of described peptides targeting EL, IL and CT regions”) and Table 1 (“Connexin Peptides”).

In some embodiments the peptides may also be used as promoieties. See, e.g. Vig, B S, et al., Amino acids as promoieties in drug design and Development. Advanced Drug Delivery Reviews 65 (10): p 1370-1385 (2013); Dhokchawle, B, et al., Promoieties Used In Prodrug Design: A Review. Indian Journal of Pharmaceutical Education 48 (2): 35-40 (2013).

The gap junction modulators or anti-connexin hemichannel blocking peptides or peptidomimetics may be unmodified, or modified as desired (e.g. to increase stability, to further stabilize peptide conformation, increase bioactivity, increase cell permeability, etc.). See e.g. DeGruyter, J N, et al., Residue-Specific Peptide Modification: A Chemist's Guide. Biochemistry 56, 30, 3863-3873 (2017); Boto, A, et al. Site-selective modification of peptide backbones. Org. Chem. Front. 8:6720-6759 (2021)(review article). Thus, for example, peptidomimetics used in methods of the invention may contain one or more modified amino acids, amino acid analogs, or may be otherwise modified to improve bioavailability or to increase penetration across the cell membrane.

Other peptide sequences known to inhibit the inter-connexin binding that can regulate connexin activity are the cytoplasmic loop of connexin 43 (amino acids 119-144) L2 peptide and subparts of the L2 peptide of connexin 43. In some embodiments, these peptides may include or exclude, for example, the nine amino acid sequence of Gap19, KQIEIKKFK (SEQ ID NO: 108); the native Gap19 sequence, DGVNVEMHLKQIEIKKFKYGIEEHGK (SEQ ID NO: 119); the His144→Glu L2 derivative of Gap19, as reported by Shibayama (Shibayama, J. et al., Biophys. J. 91, 405404063, 2006), DGVNVEMHLKQIEIKKFKYGIEEQGK (SEQ ID NO: 120); the TAT-Gap19 sequence, YGRKKRRQRRRKQIEIKKFK (SEQ ID NO: 121); the SH3 binding domain, CSSPTAPLSPMSPPGYK (SEQ ID NO: 122), or subpart thereof PTAPLSPMSPP (SEQ ID NO: 123); the C-terminal sequence of the CT9 or CT10 peptide, with or without a TAT leader sequence to increase cell penetration, for example, RPRDDEI (CT9; SEQ ID NO: 124), SRPRDDLEI (CT10; SEQ ID NO: 125), YGRKKRRQRRRSRPRDDEI (TAT-CT9; SEQ ID NO: 126), or YGRKKRRQRRRRPRDDEI (TAT-CT10; SEQ ID NO: 127). Other peptidomimetic sequences that can be included or excluded in the compositions, methods, kits or articles of manufacture disclosed herein are those reported by Dhein (Dhein, S., Naunyn-Schmiedeberg's Arch. Pharm., 350:174-184, 1994); the AAP10 peptide, H₂N-Gly-Ala-Gly-4Hyp-Pro-Tyr-CONH2 (SEQ ID NO: 128), and the ZP123 peptide (rotigapeptide), Ac-D-Tyr-Pro-D-4Hyp-Gly-D-Ala-Gly-NH2 (SEQ ID NO: 129), (Dhein, S., et al. Cell Commun. Adhes. 10, 371-378, 2013). Rotigapeptide is comprised of the D-form of the peptides for enhanced efficacy over the native L-form of the peptide.

In some embodiments, the therapeutically effective modified or unmodified peptide or peptidomimetic comprises a portion of the E1 extracellular domain of a connexin, such as the connexin 43 E1 (ESAWGDEQSAFRCNTQQPGCENVCYDKSFPISHVR; SEQ ID NO: 130) or the connexin 45 E1 (GESIYYDEQSKFVCNTEQPGCENVCYDAFAPLSHVR; SEQ ID NO:131). In some embodiments, the therapeutically effective modified or unmodified peptide or peptidomimetic comprises a portion of the E2 extracellular domain of a connexin, such as the connexin 43 E2 (LLIQWYIYGFSLSAVYTCKRDPCPHQVDCFLSRPTEKT; SEQ ID NO:132) or the connexin 45 E2 (LIGQYFLYGFQVHPFYVCSRLPCHPKIDCFISRPTEKT; SEQ ID NO: 133).

In certain embodiments, the connexin 43 modulator peptides of the present invention can be linked at the amino or carboxy terminus to a cellular internalization transporter. The cellular internalization transporter linked to connexin 43 modulator peptides of the present invention may be any internalization sequence known or newly discovered in the art, or conservative variants thereof. Non-limiting examples of cellular internalization transporters and sequences include antennapedia sequences, TAT, HIV-Tat, Penetratin, Antp-3A (Antp mutant), Buforin II, Transportan, MAP (model amphipathic peptide), K-FGF, Ku70, Prion, pVEC, Pep-1, SynB1, Pep-7, HN-1, BGSC (Bis-Guanidinium-Spermidine-Cholesterol, and BGTC (BisGuanidinium-Tren-Cholesterol). Cellular internalization transporters are useful for peptidomimetics such as Gap 19 and the aCT peptides. The sequences of exemplary cellular internalization peptides are known in the art. See, e.g., Table 65 in U.S. Pat. Nos. 10,465,188; 11,466,069.

In one embodiment, the peptide sequence CFLSRPTEKT (SEQ ID NO:116) or VDCFLSRPTEKT (SEQ ID NO:107) can be conjugated to two dodecyl groups to create a modified peptide which can modulate connexin 43, “C12-C12-Cxn43 MP” (SEQ ID NO: 358). See SEQ ID NO: 237 in U.S. Pat. No. 10,465,188. The resulting structure “C12-C12-Cxn43MP” (SEQ ID NO: is shown below.

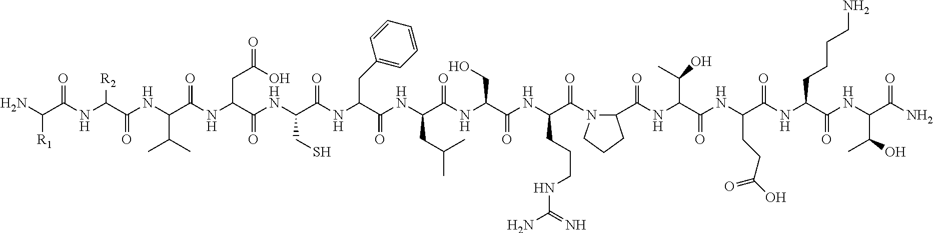

In the structure of C12-C12-Cxn43 MP (SEQ ID NO: 358), R1 and R2 can be hydrogen or alkyl groups. In some aspects, R1=R2=n-dodecyl chains.

In some embodiments, the therapeutically effective modified or unmodified peptide or peptidomimetic comprises a portion of the C-terminal domain of a connexin, such as connexin 43 or connexin 45, preferably connexin 43. Some embodiments of anti-connexin modulators useful in methods of the invention comprise the C-terminus region of connexin 43, or modified versions thereof. See e.g. O'Quinn, M P, et al., A Peptide Mimetic of the Connexin43 Carboxyl-Terminus Reduces Gap Junction Remodeling and Induced Arrhythmia Following Ventricular Injury. Circ Res. 108 (6): 704-715 (March 2011). C-terminus connexin peptidomimetic modulators, including αCT1 (alpha connexin carboxy terminus 1) peptides (also referred to as aCT1 or ACT1 peptides in publications), are described, for example, in Montgomery et al., Connexin 43-Based Therapeutics for Dermal Wound Healing Int. J. Mol. Sci. 2018, 19, 1778, and U.S. Pat. No. 8,815,556 (“Compositions and methods for tissue engineering, tissue regeneration and wound healing”). See also WO2006/069181. Preferred connexin carboxy-terminal polypeptides are connexin 43 carboxy-terminal polypeptides. Such compounds are described in US Patent Publication No. 20070042964 (“Compositions and methods for modulating connexin hemichannels”). If a connexin peptide or peptidomimetic modulator comprises a portion of an intracellular domain of a connexin (e.g. an aCT peptide, such as CT9, CT10, «CT1, etc.), the peptide may be conjugated to a cell internalization transporter including those noted or referenced herein. In some embodiments, a connexin peptidomimetic modulator useful in methods of then invention may block zona occludens (ZO-1) binding to connexin 43 and modulate connexin gap junctions and hemichannels to advantage. See FIG. 2 in Caufriez, A, et al. Peptide-based targeting of connexins and pannexins for therapeutic purposes. Expert Opinion on Drug Discovery 15 (10): 1213-1222 (2020).

In some embodiments, the connexin modulator may be a gap junction closing compound and/or hemichannel closing compound. In some embodiments, the gap junction closing compounds and hemichannel closing compounds are connexin 43 gap junction closing compounds and connexin 43 hemichannel closing compounds (e.g. a Cx43 C-terminus peptidomimetic).

Various useful peptidomimetic peptides mimic sequences of connexin extracellular regions. The first extracellular loops of Cx37, Cx40 as well Cx43 are mimicked by peptides under the Gap26 code. Gap27 and Peptide5 mimic regions of the second extracellular loop. Gap27 targets Cx32, Cx40 and Cx43, while Peptide5 is used for Cx43 inhibition. JM2, ASH3, CT9, CT10, αCT mimic the C-terminal tail of Cx43 and Gap24 reproduces a sequence of the cytoplasmic loop of Cx32. Both L2 and Gap19 also mimic the cytoplasmic loop but of Cx43.

The extracellular loops of connexin hemichannels are also good targets for peptidomimetic connexin inhibitors useful in the invention due to their accessibility in contrast to their full channel counterparts. Nevertheless, peptides containing the conserved motives QPG and SHVR (SEQ ID NO: 359) of the first extracellular loop and the SRPTEK motif (SEQ ID NO: 360) of the second extracellular loop interfere with the formation of gap junctions. This led to the development of peptide mimetics ⁴³Gap26, ^{37,40}Gap26, ³²Gap27, ⁴⁰Gap27, ⁴³Gap27 and ⁴³Peptide5 (note that the superscript in the nomenclature of these peptide analogues refers to Cx subtypes they can target).

The conserved SHVR motif (SEQ ID NO: 359) of the first extracellular loop is incorporated in the sequence of useful Gap26 peptide mimetics. The two slightly different sequences are both categorized under the Gap26 code, one targeting Cx37 and Cx40, and the other targeting only Cx43. Cells treated with either of these Gap26 peptides showed Cx hemichannel inhibition within minutes.

There are three Gap27 peptides, each targeting different Cx types, namely Cx32, Cx40 and Cx43 that are useful in compositions and methods of the invention. These peptides mimic the conserved SRPTEK motif (SEQ ID NO: 360) of the second extracellular loop, but have the same time-dependent effect on gap junction activity as Gap26. Like Gap27, Peptide5 contains the SRPTEK motif (SEQ ID NO: 360). However, the mimicked sequence of Peptide5 is shifted in the direction of the N-terminal tail in comparison to that of Gap27. Peptide5 can inhibit Cx43 hemichannels at a concentration of 5-10 μM, but incubation at higher concentration (100 μM or higher) can also leads to inhibition of gap junctions in some circumstances.

Other peptides useful in compositions and methods of the invention mimic sequences of connexin intracellular regions. The interaction between the cytoplasmic loop and C-terminal tail mediates the gating mechanism of Cx hemichannels and gap junctions. Gap junctions are in an open state when there is no interaction between the C-terminal tail and cytoplasmic loop, while such interaction is critical for Cx hemichannel opening. The CT10 peptidomimetic reproduces the last 10 amino acids of the C-terminal tail of Cx43. Inhibition of Cx43-mediated ATP release by a peptide mimetic, called TAT-L2, pinpointed its mimicked L2 region (amino acid 119 to 144) as an essential sequence of the cytoplasmic loop in the interaction with the Cx43 C-terminal tail. To date, 2 peptides mimicking the L2 region are available, namely ⁴³Gap19 and ³²Gap24. ⁴³Gap19 inhibits Cx43 hemichannel currents by binding to the C-terminal tail, thereby inhibiting the cytoplasmic loop/C-terminal tail interaction. ⁴³Gap19 has the advantage of being a selective inhibitor, as it does not affect gap junction or Panx1 channel activity. ³²Gap24 is a peptide that mimics a 13 amino acid long stretch of the L2 region of Cx32 and can also be used in methods of the invention. Cx32 is one of the ten human corneal epithelial connexin that can be usefully targeted as described herein. In vitro studies showed that Cx32 hemichannel-mediated ATP release is inhibited by ³²Gap24 at concentrations of 17 μM without affecting gap junctions.

Peptides targeting intracellular regions of Cx proteins need to access the intracellular environment. Cell-penetrating peptides for this purpose are described herein (e.g. the TAT-peptide, an oligoarginine tag and the Xentry peptide), and can be anchored to Cx-derived peptide sequences in order to enhance the uptake into the cell via endocytosis. ⁴³Gap 19 can enter the cell on its own due to the KKFK cell-translocation motif (SEQ ID NO: 361) of the L2 region. Nonetheless, comparison of the IC₅₀ (half maximal inhibitory concentration) of ⁴³Gap 19 itself (47 μM) and TAT-⁴³Gap19 (7 μM) for the inhibition of ATP release in glioma cells showed that the entry of ⁴³Gap 19 into the cell can be improved by linking to a TAT-tag. All 5 available peptides mimicking the C-terminal tail are derived from Cx43.

αCT1 mimics the last 9 amino acids of Cx43 and is linked to an antennapedia sequence that facilitates cellular internalization of the peptide mimetic. The interaction between Cx43 and the PDZ-domain of ZO-1, a region that is suggested to be involved in the regulation of Cx trafficking and gap junction assembly, is disrupted by αCT1. This interference reportedly leads to increased gap junction plaque formation and decreased Cx hemichannel activity.

If a peptide or peptidomimetic modulator comprises a portion of an intracellular domain of a connexin, the peptide may, in some embodiments, be conjugated to a cell internalization transporter and may, in some instances, block zona occludens (ZO-1) binding to connexin 43.

In some embodiments of this invention, the connexin modulator is a peptide or peptidomimetic shown in Table II below (E2 and T2 refer to the location of a peptide in, for example, the second extracellular domain or the second transmembrane domain).

TABLE II

| SEQ ID NO: | Identifier | Sequence |
|---|---|---|
| SEQ ID NO: 140 | CXT 2 | PSSRASSRASSRPRPDDLEI |
| SEQ ID NO: 141 | CXT 1 | RPRPDDLEI |
| SEQ ID NO: 142 | CXT 3 | RPRPDDLEV |
| SEQ ID NO: 143 | CXT 4 | RPRPDDVPV |
| SEQ ID NO: 144 | CXT 5 | KARSDDLSV |
| SEQ ID NO: 145 | hCx40 | QKPEVPNGVSPGHRLPHGYHSDKRRLSKASSKARSD |
| | | DLSV |
| SEQ ID NO: 146 | Antp/CXT 2 | RQPKIWFPNRRKPWKKPSSRASSRASSRPRPDDLEI |
| SEQ ID NO: 147 | Antp/CXT 2 | RQPKIWFPNRRKPWKKPSSRASSRASSRPRPDDLEI |
| SEQ ID NO: 148 | Antp/CXT 1 | RQPKIWFPNRRKPWKKRPRPDDLEI |
| SEQ ID NO: 149 | Antp/CXT 3 | RQPKIWFPNRRKPWKKRPRPDDLEV |
| SEQ ID NO: 150 | Antp/CXT 4 | RQPKIWFPNRRKPWKKRPRPDDVPV |
| SEQ ID NO: 151 | Antp/CXT 5 | RQPKIWFPNRRKPWKKKARSDDLSV |
| SEQ ID NO: 152 | conservative | RPKPDDLDI |
| | Cx43 variant | |
| SEQ ID NO: 153 | HIV-Tat/CXT | GRKKRRQRPPQRPRPDDLEI |
| | 1 | |
| SEQ ID NO: 154 | Penetratin/CXT | RQIKIWFQNRRMKWKKRPRPDDLEI |
| | 1 | |
| SEQ ID NO: 155 | Antp-3A/CXT | RQIAIWFQNRRMKWAARPRPDDLEI |
| | 1 | |
| SEQ ID NO: 156 | Tat/CXT 1 | RKKRRQRRRRPRPDDLEI |
| SEQ ID NO: 157 | Buforin II/Vnrs | TRSSRAGLQFPVGRVHRLLRKRPRPDDLEI |
| | 1 | |
| SEQ ID NO: 158 | Transportan/ | GWTLNSAGYLLGKINKALAALAKKILRPRPDDLEI |
| | CXT 1 | |
| SEQ ID NO: 169 | MAP/CXT 1 | KLALKLALKALKAALKLARPRPDDLEI |
| SEQ ID NO: 160 | K-FGF/CXT 1 | AAVALLPAVLLALLAPRPRPDDLEI |
| SEQ ID NO: 161 | Ku70/CXT 1 | VPMLKPMLKERPRPDDLEI |
| SEQ ID NO: 162 | Prion/CXT 1 | MANLGYWLLALFVTMWTDVGLCKKRPKPRPRPDDL |
| | | EI |
| SEQ ID NO: 163 | pVEC/CXT 1 | LLIILRRRIRKQAHAHSKRPRPDDLEI |
| SEQ ID NO: 164 | Pep-1/CXT 1 | KETWWETWWTEWSQPKKKRKVRPRPDDLEI |
| SEQ ID NO: 165 | SynB1/CXT 1 | RGGRLSYSRRRFSTSTGRRPRPDDLEI |
| SEQ ID NO: 166 | Pep-7/CXT 1 | SDLWEMMMVSLACQYRPRPDDLEI |
| SEQ ID NO: 167 | HN-1/CXT 1 | TSPLNIHNGQKLRPRPDDLEI |
| SEQ ID NO: 174 | SEQ-Mod4 | VDCFLSRPTE |
| SEQ ID NO: 175 | SEQ-Mod5 | VDCFLSRP |
| SEQ ID NO: 176 | SEQ-Mod6 | VDCFLS |
| SEQ ID NO: 177 | HIV-Tat/SEQ- | GRKKRRQRPPQVDCFLSRPTEKT |
| | pept5 | |
| SEQ ID NO: 178 | Penetratin/ | RQIKIWFQNRRMKWKKVDCFLSRPTEKT |
| | SEQ-pept5 | |
| SEQ ID NO: 179 | Antp-3A/SEQ- | RQIAIWFQNRRMKWAAVDCFLSRPTEKT |
| | pept5 | |
| SEQ ID NO: 180 | Tat/SEQ-pept5 | RKKRRQRRRVDCFLSRPTEKT |
| SEQ ID NO: 181 | Buforin II/ | TRSSRAGLQFPVGRVHRLLRKVDCFLSRPTEKT |
| | SEQ-pept5 | |
| SEQ ID NO: 182 | Transportan/ | GWTLNSAGYLLGKINKALAALAKKILVDCFLSRPTEK |
| | SEQ-pept5 | T |
| SEQ ID NO: 183 | MAP/SEQ- | KLALKLALKALKAALKLAVDCFLSRPTEKT |
| | pept5 | |
| SEQ ID NO: 184 | K-FGF/SEQ- | AAVALLPAVLLALLAPVDCFLSRPTEKT |
| | pept5 | |
| SEQ ID NO: 185 | Ku70/SEQ- | VPMLKPMLKEVDCFLSRPTEKT |
| | pept5 | |
| SEQ ID NO: 186 | Prion/SEQ- | MANLGYWLLALFVTMWTDVGLCKKRPKPVDCFLSR |
| | pept5 | PTEKT |
| SEQ ID NO: 187 | pVEC/SEQ- | LLIILRRRIRKQAHAHSKVDCFLSRPTEKT |
| | pept5 | |
| SEQ ID NO: 188 | Pep-1/SEQ- | KETWWETWWTEWSQPKKKRKVVDCFLSRPTEKT |
| | pept5 | |
| SEQ ID NO: 189 | SynB1/SEQ- | RGGRLSYSRRRFSTSTGRVDCFLSRPTEKT |
| | pept5 | |
| SEQ ID NO: 190 | Pep-7/SEQ- | SDLWEMMMVSLACQYVDCFLSRPTEKT |
| | pept5 | |
| SEQ ID NO: 191 | HN-1/SEQ- | TSPLNIHNGQKLVDCFLSRPTEKT |
| | pept5 | |
| SEQ ID NO: 192 | SEQ M3E2 | FEVAFLLIQWI |
| SEQ ID NO: 193 | SEQ E2a | LLIQWYIGFSL |
| SEQ ID NO: 194 | SEQ E2b | SLSAVYTCKRDPCPHQ |
| SEQ ID NO: 196 | SEQ M1E1 | LGTAVESAWGDEQ |
| SEQ ID NO: 197 | SEQ E1a | QSAFRCNTQQPG |
| SEQ ID NO: 198 | SEQ E1b | QQPGCENVCYDK |
| SEQ ID NO: 200 | SEQ E2d | KRDPCHQVDCFLSRPTEK |
| SEQ ID NO: 291 | Peptide 1 | ADCFLSRPTEKT |
| SEQ ID NO: 292 | Peptide 2 | VACFLSRPTEKT |
| SEQ ID NO: 293 | Peptide 11 | VDCFLSRPTAKT |
| SEQ ID NO: 294 | Peptide 12 | VDCFLSRPTEAT |
| SEQ ID NO: 108 | Gap 19-subpart | KQIEIKKFK |
| SEQ ID NO: 300 | Gap 19-full | DGVNVEMHLKQIEIKKFKYGIEEHGK |
| SEQ ID NO: 301 | Gap 19-deriv | DGVNVEMHLKQIEIKKFKYGIEEQGK |
| SEQ ID NO: 302 | TAT-Gap19 | YGRKKRRQRRRKQIEIKKFK |
| SEQ ID NO: 303 | SH3-full | CSSPTAPLSPMSPPGYK |
| SEQ ID NO: 304 | SH3-subpart | PTAPLSPMSPP |
| SEQ ID NO: 309 | AAP10 | H₂N-Gly-Ala-Gly-4Hyp-Pro Tyr-CONH₂ |
| SEQ ID NO: 310 | ZP123 | Ac-D-Tyr-Pro-D-4Hyp-Gly-D-Ala-Gly-NH₂ |
| SEQ ID NO: 311 | plsl/SEQ-pept5 | RVIRVWFQNKRCKDKKVDCFLSRPTEKT |
| SEQ ID NO: 312 | MGB Peptide | GALFLGFLGAAGSTMGAWSQPKKKRKVVDCFLSRPT |
| | P-beta/SEQ- | EKT |
| | pept5 | |
| SEQ ID NO: 313 | MGB Peptide | GALFLAFLAAALSLMGLWSQPKKKRRVVDCFLSRPT |
| | P-alpha/SEQ- | EKT |
| | pept5 | |
| SEQ ID NO: 343 | huCx26 | MYVFYVMYDGFSMQRLVKCNAWPCPNTVDCFVSRP |
| | | TEKT |
| SEQ ID NO: 344 | huCx30 | MYVFYFLYNGYHLPWVLKCGIDPCPNLVDCFISRPTE |
| | | KT |
| SEQ ID NO: 345 | huCx30.3 | LYIFHRLYKDYDMPRVVACSVEPCPHTVDCYISRPTE |
| | | KK |
| SEQ ID NO: 346 | huCx31 | LYLLHTLWHGFNMPRLVQCANVAPCPNIVDCYIARP |
| | | TEKK |
| SEQ ID NO: 347 | huCx31.1 | LYVFHSFYPKYILPPVVKCHADPCPNIVDCFISKPSEK |
| | | N |
| SEQ ID NO: 348 | huCx32 | MYVFYLLYPGYAMVRLVKCDVYPCPNTVDCFVSRP |
| | | TEKT |
| SEQ ID NO: 349 | huCx36 | LYGWTMEPVFVCQRAPCPYLVDCFVSRPTEKT |
| SEQ ID NO: 350 | huCx37 | LYGWTMEPVFVCQRAPCPYLVDCFVSRPTEKT |
| SEQ ID NO: 351 | huCx40.1 | GALHYFLFGFLAPKKFPCTRPPCTGVVDCYVSRPTEK |
| | | S |
| SEQ ID NO: 353 | huCx46 | IAGQYFLYGFELKPLYRCDRWPCPNTVDCFISRPTEK |
| | | T |
| SEQ ID NO: 354 | huCx46.6 | LVGQYLLYGFEVRPFFPCSRQPCPHVVDCFVSRPTEK |
| | | T |
| SEQ ID NO: 355 | huCx40 | IVGQYFIYGIFLTTLHVCRRSPCPHPVNCYVSRPTEKN |

In some embodiments the connexin 43 modulator may comprise, for example, a peptide or peptidomimetic comprising, for example SEQ ID NO: 101 (SRPTEKT). The peptide or peptidomimetic may also comprise, for example SEQ ID NO: 168 (VDCFLSRPTEKT). The peptide may contain one or more modified amino acids, amino acid analogs, or may be otherwise modified to improve bioavailability or to increase penetration across the cell membrane. For example, SEQ ID NO: 107 may be modified to obtain SEQ ID NOS: 177-191 and 311-313. In some embodiments, the peptide or peptidomimetic comprising, for example SEQ ID NO: 101 (SRPTEKT) or SEQ ID NO: 107 (VDCFLSRPTEKT) comprises from 7 to 40 amino acids or amino acid analogues and does not comprise a C-terminal peptide. In some embodiments the peptides may also be used as promoieties.

In some embodiments, the Connexin 45 modulators can be peptide or peptidomimetics comprising portions of the Connexin 45 protein that antagonize or inhibit or block connexin-connexin interactions. Exemplary peptide sequences for Connexin 45 peptides and peptidomimetic modulators are provided in Table III.

TABLE III
Sequences of Connexin 45 modulator peptides or
peptidomimetics

| SEQ ID NO. | Sequence |
|---|---|
| SEQ ID NO: | LTAVGGESIYYDEQSKFVCNTEQPGCENVCYDAFAP |
| 237 | LSHVRFWVFQ |
| SEQ ID NO: | LTAVGGESIYYDEQS |
| 238 | |
| SEQ ID NO: | DEQSKFVCNTEQP |
| 239 | |
| SEQ ID NO: | TEQPGCENVCYDA |
| 241 | |
| SEQ ID NO: | VCYDAFAPLSHVR |
| 242 | |
| SEQ ID NO: | APLSHVRFWVFQ |
| 243 | |
| SEQ ID NO: | FEVGFLIGQYF |
| 245 | |
| SEQ ID NO: | LIGQYFLYGFQV |
| 246 | |
| SEQ ID NO: | GFQVHPFYVCSRLP |
| 247 | |
| SEQ ID NO: | SRLPCHPKIDCF |
| 248 | |
| SEQ ID NO: | IDCFISRPTEKT |
| 249 | |
| SEQ ID NO: | SRPTEKTIFLL |
| 250 | |
| SEQ ID NO: | YVCSRLPCHP |
| 252 | |
| SEQ ID NO: | QVHPFYVCSRL |
| 253 | |
| SEQ ID NO: | FEVGFLIGQYFLY |
| 254 | |
| SEQ ID NO: | GQYFLYGFQVHP |
| 255 | |
| SEQ ID NO: | GFQVHPFYVCSR |
| 256 | |
| SEQ ID NO: | AVGGESIYYDEQ |
| 257 | |
| SEQ ID NO: | YDEQSKFVCNTE |
| 258 | |
| SEQ ID NO: | NTEQPGCENVCY |
| 259 | |
| SEQ ID NO: | CYDAFAPLSHVR |
| 260 | |
| SEQ ID NO: | FAPLSHVRFWVF |
| 261 | |
| SEQ ID NO: | LIGQY |
| 262 | |
| SEQ ID NO: | QVHPF |
| 263 | |
| SEQ ID NO: | YVCSR |
| 264 | |
| SEQ ID NO: | SRLPC |
| 265 | |
| SEQ ID NO: | LPCHP |
| 266 | |
| SEQ ID NO: | GESIY |
| 267 | |
| SEQ ID NO: | YDEQSK |
| 268 | |
| SEQ ID NO: | SKFVCN |
| 269 | |
| SEQ ID NO: | TEQPGCEN |
| 270 | |
| SEQ ID NO: | VCYDAFAP |
| 271 | |
| SEQ ID NO: | LSHVRFWVFQ |
| 272 | |
| SEQ ID NO: | LIQYFLYGFQVHPF |
| 273 | |
| SEQ ID NO: | VHPFYCSRLPCHP |
| 274 | |
| SEQ ID NO: | VGGESIYYDEQSKFVCNTEQPG |
| 275 | |
| SEQ ID NO: | TEQPGCENVCYDAFAPLSHVRF |
| 276 | |
| SEQ ID NO: | AFAPLSHVRFWVFQ |
| 277 | |
| SEQ ID NO: | IDCFISRPTEKTIFLL |
| 278 | |
| SEQ ID NO: | DCFISRPTEKT |
| 279 | |
| SEQ ID NO: | SRPTEKT |
| 101 | |

In some embodiments the connexin 45 modulator may comprise, for example, a peptide or peptidomimetic comprising, a portion of the E2 or C terminal domain of connexin 45, for example, comprising SEQ ID NO: 101 (SRPTEKT). The peptide or peptidomimetic may also comprise, for example SEQ ID NO: 279 (DCFISRPTEKT). In some embodiments the peptides may only be 3 amino acids in length, including SRL, PCH, LCP, CHP, IYY, SKF, QPC, VCY, APL, HVR, or longer.

When specific proteins are referred to herein, derivatives, variants, and fragments are contemplated and included. Protein derivatives and variants are well understood to those of skill in the art and can involve insertional, substitutional or deletional amino acid sequence variants known in the art.

The gap junction modulators and anti-connexin hemichannel blocking peptides or peptidomimetics are made chemically, synthetically, or otherwise manufactured.

### Connexin Hemichannel Modulators

Any modulator that is capable of eliciting a desired inhibition of the passage (e.g., transport) of molecules through a connexin gap junction and/or connexin hemichannel in ocular or corneal blood vessels, and well as connexin gap junctions and/or connexin hemichannel in the ocular or corneal epithelium, may be used in embodiments of the invention and dosed according to the one or more of the schedules described herein. Any connexin agents that modulates the passage of molecules through a gap junction or connexin hemichannel are also provided in particular embodiments (e.g., those that modulate, block or lessen the passage of molecules from the cytoplasm of a cell into an extracellular space or adjoining cell cytoplasm, including ATP). Such anti-connexin modulators may modulate the passage of molecules through a gap junction or connexin hemichannel with or without gap junction uncoupling (blocking the transport of molecules through gap junctions). Such compounds include, for example, binding proteins (e.g. scFvs, antibodies, etc.), polypeptides (e.g. peptidomimetics), and organic compounds (e.g. tonabersat and compounds of Formula I and/or Formula II) that can, for example, block the function or activity of a gap junction or a hemichannel in whole or in part (e.g. by modulating release of ATP from connexin hemichannels).

In some embodiments, the modulator used in methods of the invention is a gap junction closing or blocking compound or hemichannel closing or blocking compound (e.g. tonabersat). In some embodiments, the modulator can be a small molecule, which may also be referred to herein as an anti-connexin or a connexin or connexin gap junction or connexin hemichannel modulator. In some embodiments, methods of the invention feature the use of compounds of Formula I, for example tonabersat and/or carabersat, to directly and immediately block Cx43 hemichannels and to cause a concentration and time-dependent reduction in GJ coupling and/or hemichannel inhibition (e.g., blocking hemichannel opening and/or modulating or blocking ATP release from connexin hemichannels). Carabersat is N-[(3R,4S)-6-acetyl-3-hydroxy-2,2-dimethyl-3,4-dihydrochromen-4-yl]-4-fluorobenzamide) and is also referred to as trans-(+)-6-acetyl-4-(S)-(4-fluorobenzoylamino)-3,4-dihydro-2,2-dimethyl-2H-1-benzo[b]pyran-3R-ol,hemihydrate. Tonabersat is also known by the IUPAC name N-[(3S,4S)-6-acetyl-3-hydroxy-2,2-dimethyl-3,4-dihydrochromen-4-yl]-3-chloro-4-fluorobenzamide or (3S-cis)-N-(6-acetyl-3,4-dihydro-3-hydroxy-2,2-(dimethyl-d6)-2H-1-benzopyran-4-yl)-3-chloro-4-fluorobenzamide).

In some embodiments, the anti-connexin or connexin or connexin gap junction or connexin hemichannel modulator is a compound according to Formula I:

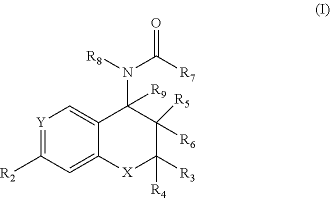

wherein Y is C—R₁;
R₁ is acetyl;
R₂ is hydrogen, C₃₋₈ cycloalkyl, C₁₋₆ alkyl optionally interrupted by oxygen or substituted by hydroxy, C₁₋₆ alkoxy or substituted aminocarbonyl, C₁₋₆ alkylcarbonyl, C₁₋₆alkoxycarbonyl, C₁₋₆ alkylcarbonyloxy, C₁₋₆ alkoxy, nitro, cyano, halo, trifluoromethyl, or CF₃—S—; or a group CF₃-A-, where A is —CF₂—, —CO—, —CH₂—, CH(OH), SO₂, SO, CH₂—O, or CONH; or a group CF₂H-A′- where A′ is oxygen, sulphur, SO, SO₂, CF₂ or CFH; trifluoromethoxy, C₁₋₆ alkylsulphinyl, perfluoro C₂₋₆ alkylsulphonyl, C₁₋₆ alkylsulphonyl, C₁₋₆alkoxysulphinyl, C₁₋₆ alkoxysulphonyl, aryl, heteroaryl, arylcarbonyl, heteroarylcarbonyl, phosphono, arylcarbonyloxy, heteroarylcarbonyloxy, arylsulphinyl, heteroarylsulphinyl, arylsulphonyl, or heteroarylsulphonyl in which any aromatic moiety is optionally substituted, C₁₋₆alkylcarbonylamino, C₁₋₆ alkoxycarbonylamino, C₁₋₆ alkyl-thiocarbonyl, C₁₋₆ alkoxy-thiocarbonyl, C₁₋₆ alkyl-thiocarbonyloxy, 1-mercapto C₂₋₇ alkyl, formyl, or aminosulphinyl, aminosulphonyl or aminocarbonyl, in which any amino moiety is optionally substituted by one or two C₁₋₆ alkyl groups, or C₁₋₆ alkylsulphinylamino, C₁₋₆ alkylsulphonylamino, C₁₋₆alkoxysulphinylamino or C₁₋₆ alkoxysulphonylamino, or ethylenyl terminally substituted by C₁₋₆alkylcarbonyl, nitro or cyano, or —C(C₁₋₆ alkyl)NOH or —C(C₁₋₆ alkyl)NNH₂; or amino optionally substituted by one or two C₁₋₆alkyl or by C₂₋₇ alkanoyl;
one of R₃ and R₄ is hydrogen or C₁₋₄ alkyl and the other is C₁₋₄ alkyl, CF₃, or CH₂X^{a}, where X^{a} is fluoro, chloro, bromo, iodo, C₁₋₄ alkoxy, hydroxy, C₁₋₄ alkylcarbonyloxy, —S—C₁₋₄alkyl, nitro, amino optionally substituted by one or two C₁₋₄ alkyl groups, cyano or C₁₋₄alkoxycarbonyl; or R₃ and R₄ together are C₂₋₅ polymethylene optionally substituted by C₁₋₄alkyl;
R₅ is C₁₋₆ alkylcarbonyloxy, benzoyloxy, ONO₂, benzyloxy, phenyloxy or C₁₋₄alkoxy and R₆ and R₉ are hydrogen or R₅ is hydroxy and Rois hydrogen or C₁₋₂ alkyl and Rois hydrogen;
R₇ is heteroaryl or phenyl, both of which are optionally substituted one or more times independently with a group or atom selected from chloro, fluoro, bromo, iodo, nitro, amino optionally substituted once or twice by C₁₋₄ alkyl, cyano, azido, C₁₋₄ alkoxy, trifluoromethoxy and trifluoromethyl;
R₈ is hydrogen, C₁₋₆ alkyl, OR₁₁ or NHCOR₁₀ wherein R₁₁ is hydrogen, C₁₋₆ alkyl, formyl, C₁₋₆ alkanoyl, aroyl or aryl-C₁₋₆ alkyl and R₁₀ is hydrogen, C₁₋₆ alkyl, C₁₋₆ alkoxy, mono or di C₁₋₆ alkyl amino, amino, amino-C₁₋₆ alkyl, hydroxy-C₁₋₆ alkyl, halo-C₁₋₆ alkyl, C₁₋₆ acyloxy-C₁₋₆alkyl, C₁₋₆ alkoxycarbonyl-C₁₋₆ alkyl, aryl or heteroaryl; the R₈—N—CO—R₇ group being cis to the R₅ group;
and X is oxygen or NR₁₂ where R₁₂ is hydrogen or C₁₋₆alkyl.

For any of the Markush groups set forth above, in some embodiments, each group can include or exclude any of the species listed for that group.

In some embodiments, the small molecule connexin modulator can be Tonabersat, carabersat, or SB-204269. SB-204269 is also known as (trans-(+)-6-acetyl-4S-(4-fluorobenzoylamino)-3,4-dihydro-2,2-dimethyl-2H-benzo[b]pyran-3R-ol). Carabersat is also as N-[(3R,4S)-6-Acetyl-3-hydroxy-2,2-dimethyl-3,4-dihydro-2H-chromen-4-yl]-4-known fluorobenzamide. Tonabersat is also known as N-(6-Acetyl-3-hydroxy-2,2-dimethyl-3,4-dihydro-2H-chromen-4-yl)-3-chloro-4-fluorobenzamide.

For any of the Markush groups set forth above, that group can include or exclude any of the species listed for that group.

In some embodiments, the modulator may be a pro-drug of any the compounds for use in this invention. In one aspect the connexin modulator pro-drug of this invention may be a compound of Formula II:

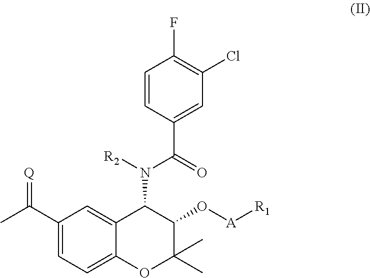

wherein
Q is O or an oxime,
R₂ is H,
A is a direct bond, —C(O)O*—, —C(R₃)(R₄)O*—, —C(O)O—C(R₃)(R₄)O*—, or —C(R₃)(R₄)OC(O)O*— wherein the atom marked * is directly connected to R₁,
R₃ and R₄ are selected independently from H, fluoro, C₁₋₄ alkyl, and C₁₋₄ fluoroalkyl, or R₃ and R₄ together with the atom to which they are attached form a cyclopropyl group,
R₁ is selected from groups [1], [2], [2A], [3], [4], [5] and [6] wherein the atom marked ** is directly connected to A:

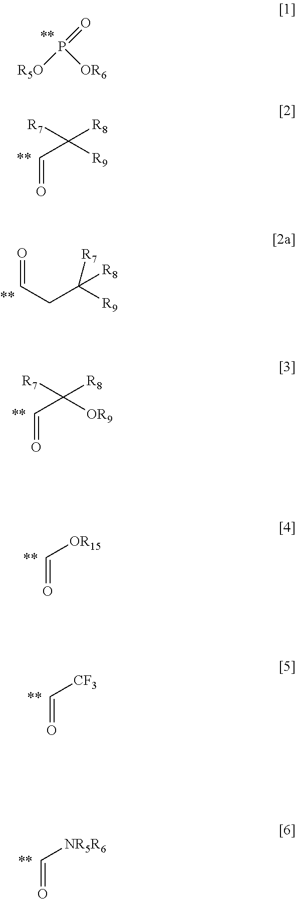

wherein
R₅ and R₆ are each independently selected from H, C₁₋₄ alkyl, C₁₋₄ fluoroalkyl, and benzyl;
R₇ is independently selected from H, C₁₋₄ alkyl, and C₁₋₄ fluoroalkyl;
R₈ is selected from:
(i) H, C₁₋₄ alkyl, or C₁₋₄ fluoroalkyl,
(ii) the side chain of a natural or unnatural alpha-amino acid, or a peptide as described herein, and
(iii) biotin or chemically linked to biotin;
R₉ is selected from H, —N(R₁₁)(R₁₂), —N⁺(R₁₁)(R₁₂)(R₁₃)X⁻, and —N(R₁₁)C(O)R₁₄;
wherein R₁₁, R₁₂, and R₁₃ are independently selected from H, C₁₋₄ alkyl, and C₁₋₄ fluoroalkyl,
R₁₄ is H, C₁₋₄ alkyl, or C₁₋₄ fluoroalkyl,
R₁₅ is selected from C₁₋₄ alkyl and C₁₋₄ fluoroalkyl, and
X⁻ is a pharmaceutically acceptable anion.
In some embodiments,
R₂ is B—R₂₁ wherein,
B is a direct bond, —C(O)O*—, —C(R₂₃)(R₂₄)O*, C(O)OC(R₂₃)(R₂₄)*, or
—C(R₂₃)(R₂₄)OC(O)O* wherein the atom marked * is directly connected to R₂₁,
R₂₃ and R₂₄ are selected independently from H, fluoro, C₁₋₄ alkyl, and C₁₋₄ fluoroalkyl,
R₂₁ is selected from groups [21], [22], [22A], [23], [24], and wherein the atom marked ** is directly connected to B:

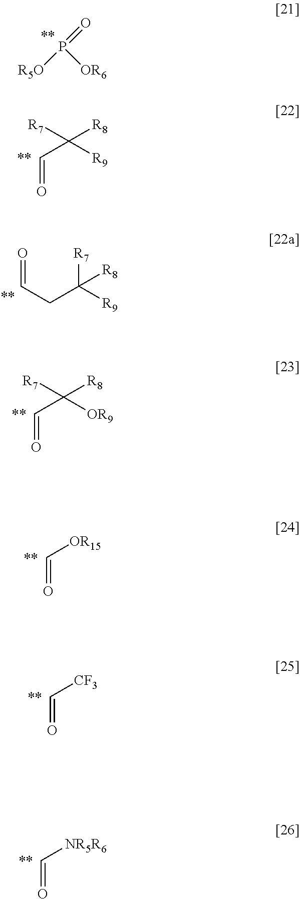

wherein R₅, R₆, R₇, R₈, R₉, and R₁₅ are as defined herein.

For any of the Formula II Markush groups set forth above, that group can include or exclude any of the species listed for that group.

In some embodiments, the peptide as described herein can be a connexin modulator, calmodulin modulator, or pannexin modulator.

In some embodiments,
Q is an oxime of formula ═NOR₄₃, wherein R₄₃ is
(i) selected from H, C₁₋₄ fluoroalkyl or optionally substituted C₁₋₄ alkyl, and
(ii) -A₃₀₀-R₃₀₀ wherein
A₃₀₀ is a direct bond, —C(O)O*—, —C(R₃)(R₄)O*—, —C(O)O—C(R₃)(R₄)O*—, or —C(R₃)(R₄)OC(O)O*— wherein the atom marked * is directly connected to R₃₀₀,
R₃ and R₄ are selected independently from H, fluoro, C₁₋₄ alkyl, and C₁₋₄ fluoroalkyl, or R₃ and R₄ together with the atom to which they are attached form a cyclopropyl group,
R₃₀₀ is selected from groups [1], [2], [2A], [3], [4], [5] and [6] wherein the atom marked ** is directly connected to A₃₀₀:

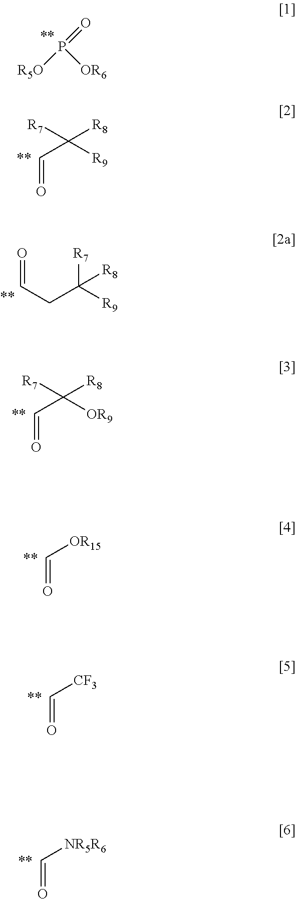

wherein R₅ and R₆ are each independently selected from H, C₁₋₄ alkyl, C₁₋₄ fluoroalkyl, and benzyl;
R₇ is independently selected from H, C₁₋₄ alkyl, and C₁₋₄ fluoroalkyl;
R₈ is selected from:
(iii) H, C₁₋₄ alkyl, or C₁₋₄ fluoroalkyl,
(iv) the side chain of a natural alpha-amino acid, and
(v) biotin or chemically linked to biotin;
R₉ is selected from H, —N(R₁₁)(R₁₂), —N⁺(R₁₁)(R₁₂)(R₁₃) X, and —N(R₁₁)C(O)R₁₄;
wherein R₁₁, R₁₂, and R₁₃ are independently selected from H, C₁₋₄ alkyl, and C₁₋₄ fluoroalkyl,
R₁₄ is H, C₁₋₄ alkyl, or C₁₋₄ fluoroalkyl,
R₁₅ is selected from C₁₋₄ alkyl and C₁₋₄ fluoroalkyl, and
X⁻ is a pharmaceutically acceptable anion.

In one embodiment of Formula II R₄₃, is C₁₋₄ alkyl optionally substituted with a phosphate group (P(O) OR₆₁R₆₂). In an example of such an embodiment OR₄₃ is —OCH₂P(O)OR₆₁OR₆₂, wherein R₆₁ and R₆₂ are independently H or C₁₋₄ alkyl.

In another embodiment of Formula II R₄₃ is an amino acid derivative having the structure C(O)CH(R₁₀₀)NH₂ wherein the group R₁₀₀ is the side chain of a natural or unnatural amino acid or a peptide as described herein.

In some embodiments, the natural amino acid is selected from one of the 22 canonical amino acids. In some embodiments, the unnatural amino acid is selected from any amino acid which is not one of the 22 canonical amino acids. In some embodiments, the unnatural amino acid is selected from: (cis)-3-Aminobicyclo[2.2.1]heptane-2-carboxylic acid hydrochloride, exo-cis-3-Aminobicyclo[2.2.1]hept-5-ene-2-carboxylic acid hydrochloride, cis-2-Amino-2-methylcyclohexanecarboxylic acid hydrochloride, (R)-2-(Boc-amino) octanedioic acid, Boc-4-(Fmoc-amino)-L-phenylalanine, Boc-(2-indanyl)-Gly-OH, (R)-4-Boc-3-morpholineacetic acid, (S)-4-Boc-3-morpholineacetic acid, Boc-pentafluoro-D-phenylalanine, Boc-pentafluoro-L-phenylalanine, Boc-Phe(2-Br)—OH, Boc-Phe(4-Br)—OH, Boc-D-Phe(4-Br)—OH, Boc-D-Phe(3-Cl)—OH, Boc-Phe(4-NH₂)—OH, Boc-Phe(3,5-F2)-OH, 2-(4-Boc-piperazino)-2-(2-fluorophenyl) acetic acid, 2-(4-Boc-piperazino)-2-(4-fluorophenyl) acetic acid, 2-(4-Boc-piperazino)-2-phenylacetic acid, 2-(4-Boc-piperazino)-2-(3-pyridyl) acetic acid, penicillamine, thialysine. quisqualic acid, canavanine. azetidine-2-carboxylic acid, Carboxyglutamic acid, Hydroxyproline, Hypusine, and Pyroglutamic acid.

In one embodiment of Formula II OR₄₃ is —OC(O)CH(CH(CH₃)₂)NH₂.

For any of the Formula II Markush groups set forth above, in some embodiments, each group can include or exclude any of the species listed for that group.

In some embodiments “promoiety” refers to a species acting as a protecting group which masks a functional group within an active agent, thereby converting the active agent into a pro-drug. The active agent may be any of the modulators or ocular therapeutics disclosed herein. Typically, the promoiety will be attached to the drug via bond(s) that are cleaved by enzymatic or non-enzymatic means in vivo, thereby converting the pro-drug into its active form. In some embodiments the promoiety may also be an active agent. In some embodiments the promoiety may be bound to a connexin modulator, a connexin gap junction modulator, or a connexin hemichannel modulator. In some embodiments the promoiety may be bound to any of the polynucleotides, peptides or peptidomimetics, small molecule antagonists and/or other treatments disclosed herein. In some embodiments the promoeity may be bound to a compound of Formula I. In some embodiments the pro-drug may be a compound of Formula II.

In some embodiments the promoiety may be any peptidomimetic or peptide antagonist of this disclosure. In some embodiments, the promoeity is a single amino acid which is optionally protected on its functional groups. In some embodiments, the promoeity is a targeting species. In some embodiments, the promoeity is a substrate for an influx or efflux transporters on the cell membrane, for example those described in Gaudana, R. et al. The AAPS Journal, 12:3, 348-360 (2012). The promoeity can be, for example, chemically-linked biotin. The promoeity can be, for example, chemically-linked D-serine.

In some embodiments, compounds of Formula I or Formula II, e.g., tonabersat, carabersat or analogues thereof, are nonionic, are in the form of a free base, a free acid, or a pharmaceutically acceptable salt. By way of example, a pharmaceutically acceptable salt includes a hydrochloride salt and salts derived from acid including, but not limited to, hydrobromic acid, hydrochloric acid, phosphoric acid, acetic acid, fumaric acid, maleic acid, salicylic acid, citric acid, oxalic acid, lactic acid, malic acid, succinic acid, methanesulphonic acid and p-toluene sulphonic acid, a salt of itself. In one embodiment, the salt is a hydrochloride salt. In one embodiment, the salt is a succinate salt.

In other embodiments, one or more polymorph, one or more isomer, and/or one or more solvate of a compound of Formula I or Formula II, e.g., tonabersat, carabersat or analogues thereof, may be used.

### Other Connexin 43 Modulators

In addition to connexin antisense (e.g. lufepirsen), connexin peptidomimetics (e.g., Peptide5, XG19, etc.) and connexin hemichannel antagonists (e.g. tonabersat), connexin binding proteins, including antibodies, antigen-binding antibody fragments, and the like, are also suitable connexin modulators for use in methods of the invention and dosed with a therapeutically effective amount according to the one or more of the dosing schedules described herein. Binding proteins include, for example, monoclonal antibodies, polyclonal antibodies, antibody fragments (including, for example, Fab, F(ab′)2 and Fv fragments; single chain antibodies; single chain Fvs; and single chain binding molecules such as those comprising, for example, a binding domain, hinge, CH2 and CH3 domains, recombinant antibodies, and antibody fragments which are capable of binding an antigenic determinant (i.e., that portion of a molecule, generally referred to as an epitope) that makes contact with a particular antibody or other binding molecule. These binding proteins, including antibodies, anti-binding antibody fragments, and so on, may be chimeric or humanized or otherwise made to be less immunogenic in the subject to whom they are to be administered, and may be synthesized, produced recombinantly, or produced in expression libraries. Any binding molecule known in the art or later discovered is envisioned, such as those referenced herein and/or described in greater detail in the art. For example, binding proteins include not only antibodies, and the like, but also ligands, receptors, peptidomimetics, or other binding fragments or molecules (for example, produced by phage display) that bind to a target (e.g., a connexin protein or connexin hemichannel epitope). Methods of synthesizing antibodies and binding fragments as well as peptides and polypeptides, including peptidomimetics and peptide analogs can also be performed using suitable methods. See e.g. Lihu Yang et al., Proc. Natl. Acad. Sci. U.S.A., 1; 95 (18): 10836-10841 (Sep. 1 1998); Harlow and Lane (1988) “Antibodies: A Laboratory Manuel” Cold Spring Harbor Publications, New York; Harlow and Lane (1999) “Using Antibodies” A Laboratory Manuel, Cold Spring Harbor Publications, New York.

Connexin modulators also include antibodies and binding fragments (e.g. scFvs, human V_{H} or V_{L} domains, humanized camelid V_{HH} domains, Ig_{NAR} single domains, etc.) thereof that bind connexin protein, and connexin peptides and polypeptides, including peptidomimetics and peptide analogs of connexin that modulate hemichannel or gap junction activity or function, and other gap junction blocking agents and gap junction protein phosphorylating agents. Connexin protein peptides and polypeptides may, for example, bind to connexin protein to inhibit its function, or may inhibit connexin function by mimicking regions of connexin protein to inhibit or disrupt its binding to other gap junction proteins. Strategies known in the art may be used to improve the naturally short half-life of antibody fragments, including PEGylation, the use of repeating peptide sequences, polysialylation, albumin or IgG binding or fusions, and other approaches.

Binding molecules will generally have a desired specificity, including but not limited to binding specificity, and desired affinity. Affinity, for example, may be a Kₐ of greater than or equal to about 10⁴ M⁻¹, greater than or equal to about 10⁶ M⁻¹, greater than or equal to about 10⁷ M⁻¹, greater than or equal to about 10⁸ M⁻¹. Affinities of even greater than about 10⁸ M⁻¹ are suitable, such as affinities equal to or greater than about 10⁹ M⁻¹, about 10¹⁰ M⁻¹, about 10¹¹ M⁻¹, and about 10¹² M⁻¹. Affinities of binding proteins useful to treat subjects according to the present invention can be readily determined using conventional techniques, for example those described by Scatchard, et al., 1949 Ann. N.Y. Acad. Sci. 51:660.

Other compounds used for modulating, blocking or closing gap junctions (e.g. phosphorylating connexin 43 tyrosine and/or serine residue) have been reported in U.S. Pat. Nos. 7,153,822 and 7,250,397.

### Connexin Modulator Combinations and Combined Administration

Methods, uses and compositions of the invention may comprise the use of a combination of two or more connexin modulators, for example, connexin antisense modulators (e.g. lufepirsen), connexin peptidomimetics (e.g. Peptide5, Gap19, XG19), and small molecule (e.g. compounds of Formula I, such as tonabersat and/or prodrugs thereof, including compounds of Formula II). Two or more connexin modulators may be administered alone or together. In some embodiments, two or more separate pharmaceutical compositions that each contain one or more connexin modulators are provided for administration. Pharmaceutical compositions are also provided for co-administration in the form of a combined preparation, for example, as an admixture of two or more modulators, e.g. gap junction, connexin and/or hemichannel modulators which may be modified or unmodified, for example one or more gap junction, connexin and/or hemichannel modulator polynucleotides and one or more gap junction, connexin and/or hemichannel modulator peptides or peptidomimetics, and optionally one or more small molecule hemichannel blockers or inhibitors that can reduce hemichannel opening and function.

Treatment of a subject for the ocular conditions described herein with one or more pharmaceutical compositions of the invention, e.g., an anti-connexin antisense and a gap junction modulator such as a connexin hemichannel blocking agent, e.g., a peptide or peptidomimetic, or a first anti-connexin agent and a second anti-connexin agent, may comprise their simultaneous, separate, sequential or sustained administration.

The term “a combined preparation” includes a “kit of parts” or “article of manufacture” in the sense that the combination partners as defined above can be dosed independently or by use of different fixed combinations with distinguished amounts of the combination partners (a) and (b), i.e. simultaneously, separately or sequentially, whether in pharmaceutical form (e.g. topical or oral) or dressing/matrix form (e.g. connexin modulator-impregnated bandage contact lens) or both in accordance with the methods, doses and Day-dosing schedules of the invention. The parts of the kit can then, for example, be administered simultaneously or chronologically staggered, that is at different time points and with equal or different time intervals for any part of the kit of parts.

In one embodiment a combined preparation is administered, wherein two or more separate connexin modulator compositions are administered to a subject in accordance with the methods, doses and dosing schedules of the invention, wherein the first composition comprises a therapeutically effective amount of modulator, such as a gap junction, connexin modulator, e.g., an anti-connexin 43 polynucleotide, peptide, or peptidomimetic, or a hemichannel closing compound, and the second composition comprises a therapeutically effective amount of a second modulator, such as a gap junction, hemichannel and/or connexin modulator, e.g., an anti-connexin polynucleotide, peptide, or peptidomimetic, a hemichannel closing compound and/or an ocular treatment agent. In another embodiment a third composition is administered comprising one or more anti-connexin polynucleotides, peptides, or peptidomimetics, a hemichannel closing compound and/or an ocular treatment agent.

The modulators, including connexin, connexin hemichannel and connexin gap junction modulators, can be dosed, administered, or formulated in methods of the invention as described herein.

### Doses and Dose Regimens

Examples of effective doses that may be used for the treatment of non-healing ocular surface defects or disorders (e.g. ocular PEDs), non-healing corneal defects or disorders (e.g. PCEDs), and other ocular diseases, disorders and conditions (e.g. uveitis, blepharitis, Sjögren's syndrome-associated keratoconjunctivitis sicca, Fuchs' dystrophy, and all forms of dry eye disease (e.g., evaporative dry eye, aqueous-deficient dry eye, and decreased wettability dry eye) and like or similar or related diseases, disorders and conditions, are described and claimed herein. In some embodiments, the therapeutically effective amount of the modulator, e.g. connexin modulator, for example a connexin 43 modulator, connexin 43 gap junction modulator and/or connexin 43 hemichannel modulator, which is effective in methods of the invention, comprises a composition having about, or at least about, 0.1 mg, 0.2 mg or 0.3 mg of a connexin modulator (e.g. lufepirsen), including doses of about (or at least about) 0.18 mg and about (or at least about) 0.018 mg of a connexin modulator (e.g. lufepirsen), or any amount within or between any two of these recited dosages. Other effective doses that are effective in methods of the invention for the treatment of ocular surface defects or disorders (e.g. ocular PEDs and PCEDs) include about, or at least about, 1.0 mg of a connexin modulator, for example, a connexin 43 modulator, a connexin 43 gap junction modulator and/or a connexin 43 hemichannel modulator (e.g. lufepirsen).

In some embodiments, the therapeutically effective amount of the modulator, e.g. connexin modulator, for example a connexin 43 modulator, connexin 43 gap junction modulator and/or connexin 43 hemichannel modulator, which is effective in methods of the invention, comprises a composition that is about, or at least about, 2 micromolar (2 μM), 5 micromolar (5 μM), 10 micromolar (10 μM), 15 micromolar (15 μM), or 20 micromolar (20 μM), including doses of about (or at least about) 1.9 micromolar (1.9 μM) or 1.9405 micromolar (1.9405 μM), and about (or at least about) 19 to 19.4 micromolar or 19.405 micromolar (19 to 19.4 μM or 19.405 μM), of a connexin modulator (e.g. lufepirsen), or any amount within or between any two of these recited dosages. Other effective doses that are effective in methods of the invention for the treatment of ocular surface defects or disorders (e.g. ocular PEDs and PCEDs) include about, or at least about, a composition comprising 30 micromolar (30 μM) or 40 micromolar (40 μM) of a connexin modulator, for example, a connexin 43 modulator, a connexin 43 gap junction modulator and/or a connexin 43 hemichannel modulator (e.g. lufepirsen).

In some methods of the invention, the connexin modulator administered to the non-healing surface defect or disorder in a subject is 0.06% lufepirsen. In some embodiments, the connexin modulator administered to a subject with a non-healing surface defect or disorder is 0.006% lufepirsen. In some embodiments, the connexin modulator administered is at least about 0.06% lufepirsen. In some embodiments, the connexin modulator administered to a subject with a non-healing surface defect or disorder is at least about 0.006% lufepirsen. In some embodiments, administration is to a PED or PCED. In some embodiments, a composition comprising or consisting essentially of a pharmaceutically acceptable carrier and about 0.06% lufepirsen, about 0.006% lufepirsen, at least about 0.06% lufepirsen or least about 0.006% lufepirsen is administered to heal the non-healing surface defect or disorder in a subject. Lufepirsen doses described herein and below may be prepared as described in Example 1.

In some embodiments, the non-healing surface defect or disorder is treated with composition comprising or consisting essentially of 0.6 mg/mL lufepirsen. In some embodiments of the present invention, the non-healing surface defect or disorder is treated with composition comprising or consisting essentially of 0.06 mg/mL lufepirsen. In some embodiments, the composition comprises or consists essentially of at least about 0.6 mg/mL lufepirsen. In some embodiments, the composition comprises or consists essentially of at least about 0.06 mg/ml lufepirsen. In some embodiments, administration is to a PED or PCED.

In some embodiments, the non-healing surface defect or disorder in a subject is treated with a composition comprising or consisting essentially of about 0.018 to about 0.18 mg of lufepirsen in the regimens described herein. In some embodiments, each dose of lufepirsen applied to the non-healing surface defect or disorder in or on the subject comprises or consists essentially of at least about 0.18 mg of lufepirsen or at least about 0.18 mg of lufepirsen in the regimens described herein. In some embodiments of the present invention, the non-healing surface defect or disorder is treated with composition comprising or consisting essentially of from about or at least about 0.01 mg, about or at least about 0.018 mg, about or at least about 0.18 mg to about or at least about 0.2 mg of lufepirsen, or from about or at least about 0.18 mg or 0.20 mg or 0.50 mg to about or at least about 1 mg of lufepirsen of lufepirsen in the regimens described herein. In some embodiments, each dose of lufepirsen applied to the non-healing surface defect or disorder comprises or consists essentially of at least about 0.15 mg, at least about 0.2 mg, at least about 0.25 mg, at least about 0.3 mg, at least about 0.35 mg, at least about 0.4 mg, at least about 0.45 mg or at least about 0.5 mg of lufepirsen in the regimens described herein. In some embodiments, administration is to a PED or PCED.

These doses are also useful for the treatment of uveitis, blepharitis, Sjögren's syndrome-associated keratoconjunctivitis sicca, Fuchs' dystrophy, and all forms of dry eye disease (e.g., evaporative dry eye, aqueous-deficient dry eye, and decreased wettability dry eye) and like or similar or related diseases, disorders and conditions.

In some embodiments, the above noted doses are administered topically to the eye of a subject with a non-healing or persistent ocular or corneal surface defect or disorder. In some embodiments, the doses referenced herein are administered intraocularly, including by injection (e.g., intravitreal, intracameral, etc.). Connexin modulators administered by routes other than topical (e.g. oral or parenteral) will be adjusted as needed to approximate these topical doses.

In some embodiments, a dose of lufepirsen (or other connexin antisense or connexin modulator) is/are administered to the non-healing surface defect or disorder in a single dose, or in two or more divided doses, e.g., for twice daily administration in up to 6 doses administered on Days 1, 2 and 14, for twice daily administration to the non-healing surface defect or disorder in up to 8 doses administered on Days 1, 2, 14 and 28, etc. In other embodiments, about or at least about 0.1 to 0.5 mg or at least about 1 mg of lufepirsen is administered in single or divided doses, e.g., for twice daily administration, in up to 14 doses administered on Days 1, 2 and 14, and one or more of Day 7, Day 21, Day 28 and/or Day 35, for example. In some embodiments, administration is to a PED or PCED in a subject.

In some embodiments, connexin modulator doses, e.g. lufepirsen, are administered on Days 1, 2, 14 and 21.

In some embodiments, connexin modulator doses, e.g. lufepirsen, are administered on Days 1, 2, 7, 14 and 21.

In some embodiments, connexin modulator doses, e.g. lufepirsen, are administered on Days 1, 2, 7, 14, 21 and 28.

In some embodiments, 3 doses of a connexin modulator (e.g. lufepirsen) are administered over 14 days to treat a non-healing or persistent ocular or corneal surface defect in a subject, including, for example, on Days 1 and 2. In some embodiments, 4 doses of a connexin modulator (e.g. lufepirsen) are administered over about 28 days. In some embodiments, 5 doses of a connexin modulator (e.g. lufepirsen) are administered over about 28 to about 35 days. In some embodiments, 6-7 doses of a connexin modulator (e.g. lufepirsen) are administered over about 35 days. For example, in one embodiment, a connexin modulator (e.g. lufepirsen) is administered 4 times over Days 1 and 2, on about Day 14 and 28. In another embodiment, a connexin modulator (e.g. lufepirsen) is administered 5 times over Days 1 and 2, on about Day 14, on about Day 28 and on about Day 35. In another embodiment, a connexin modulator (e.g. lufepirsen) is administered 5 times over Days 1 and 2, on about Day 7, on about Day 14 and on about Day 28. In another embodiment, a connexin modulator (e.g. lufepirsen) is administered 6 times over Days 1 and 2, on about Day 7, on about Day 14, on about Day 21 and on about Day 28. In another embodiment, a connexin modulator (e.g. lufepirsen) is administered 7 times over Days 1 and 2, on about Day 7, on about Day 14, on about Day 21 and on about Day 28. Reference to “administration” here refers, of course, to administration of a connexin modulator in a single dose or in divided doses.

Wherever a dose of a connexin modulator is not specified in a dose regimen or method of the invention for the treatment of a non-healing ocular or corneal surface defect or disorder (e.g. a dose of lufepirsen or another connexin expression modulator, a connexin peptidomimetic and/or a hemichannel blocker), dosing with a therapeutically effective amount of a connexin modulator(s) is intended.

In some embodiments, one or more connexin modulators is/are administered using one or more of the dose regimens described herein to a subject having an ocular diseases, disorders and condition selected from the group consisting of uveitis, blepharitis, Sjögren's syndrome-associated keratoconjunctivitis sicca, Fuchs' dystrophy, and all forms of dry eye disease (e.g., evaporative dry eye, aqueous-deficient dry eye, and decreased wettability dry eye).

Wherever a dose of a connexin modulator is specified for Day 7, 14, 21, 28 or 35 in a dose regimen or method of the invention for the treatment of a non-healing ocular or corneal surface defect or disorder (e.g. a dose of lufepirsen or another connexin expression modulator, a connexin peptidomimetic and/or a hemichannel blocker), or other disease, disorder or condition, dosing on about that day with a therapeutically effective amount of a connexin modulator(s) is intended.

Administration of connexin modulators (e.g. lufepirsen, etc.) and pharmaceutical compositions comprising or consisting essentially of a connexin modulator(s) is provided for in certain dose regimens. In one embodiment, a composition comprising one or more gap junction, hemichannel and/or connexin modulator polynucleotides (and/or other connexin modulator(s), e.g., a peptidomimetic or small molecule connexin modulator) is administered on treatment Day and treatment Day 2, followed by administration at Day 14. In another embodiment, the composition is administered on treatment Day 1 and treatment Day 2, followed by administration at Day 14 and Day 28. In another embodiment, the composition is administered on Day 1 and Day 2, followed by administration at Day 14, Day 28 and Day 35. In other embodiments of these dosing regimens, the composition is also optionally administered on Day 7 and/or Day 21.

In some embodiments the disease, disorder or condition to be treated is a non-healing ocular surface defect or disorder is a persistent epithelial defect (PED). In some embodiments the non-healing ocular surface defect or disorder is a persistent corneal epithelial defect (PCED). In some embodiments the non-healing ocular surface defect or disorder is a corneal ulcer. In some embodiments the disease, disorder or condition to be treated is selected from the group consisting of uveitis, blepharitis, Sjögren's syndrome-associated keratoconjunctivitis sicca, Fuchs' dystrophy, and all forms of dry eye disease (e.g., evaporative dry eye, aqueous-deficient dry eye, and decreased wettability dry eye). In methods of the invention, administering a therapeutically effective amount of at least one connexin modulator in pulse-dose format, with administrations on Day 1 and Day 2, followed by Day 14 and optionally on or on about Day 28 (and also optionally administration on or on about Day 35) is effective for healing the non-healing ocular surface defect or disorder, or other disease, disorder or condition. See Example 2, below. Connexin modulator doses may also be administered on Day 7 and/or Day 21. In some embodiments, the connexin modulator is a connexin 43 modulator (e.g. a connexin expression modulator, such as an antisense connexin expression modulator, including a Cx43 antisense). Other embodiments include other connexin 43 gap junction modulators and connexin hemichannel blockers or modulators (e.g. peptidomimetics and small molecules).

In some embodiments, doses of a connexin modulator (e.g. lufepirsen or another modulator) are administered at Days 1, 2 and 14. In some embodiments, connexin modulator doses, e.g. lufepirsen, are administered on Days, 1, 2, 14 and 28. In some embodiments, connexin modulator doses, e.g. lufepirsen, are administered on Days, 1, 2, 14, 28 and 35. In some embodiments, connexin modulator doses, e.g. lufepirsen, are administered on Days, 1, 2, 7, 14, and 21. In some embodiments, connexin modulator doses, e.g. lufepirsen, are administered on Days, 1, 2, 7, 14, 21, and 28. In some embodiments, connexin modulator doses, e.g. lufepirsen, are administered on Days, 1, 2, 7, 14, 21, 28, and 35. In some embodiments, a dose is administered after Day 35. In some embodiments, administration using the doses and dose regimens described is to a PED or PCED. In some embodiments, the connexin modulator comprises a connexin antisense molecule (e.g. lufepirsen). In some embodiments, the connexin modulator comprises a connexin peptidomimetic (e.g., Peptide5, Gap19, XG19, Gap26, Gap27, aCT1, etc.). In some embodiments, the connexin modulator comprises a small molecule connexin hemichannel blocker (e.g., tonabersat). In some embodiments, the connexin modulator is a connexin 43 modulator.

All descriptions with respect to dosing, unless otherwise expressly stated, apply to the modulators of the invention, including connexin modulators, connexin gap junction modulators and connexin hemichannel modulators. All descriptions with respect to dosing, unless otherwise expressly stated, also apply to all indications described or referenced herein for treatment, including ocular surface defects and corneal surface defects. All descriptions with respect to dosing, unless otherwise expressly stated, apply to the treatment of uveitis, blepharitis, Sjögren's syndrome-associated keratoconjunctivitis sicca, Fuchs' dystrophy, and all forms of dry eye disease (e.g., evaporative dry eye, aqueous-deficient dry eye, and decreased wettability dry eye).

### Formulations

The pharmaceutical compositions of this invention include various delivery forms and formulations, as desired or appropriate, including formulations for topical administration, as well as forms and formulations for drug administration that are appropriate for systemic administration (e.g., oral and enteral, etc.), parenteral administration (e.g., injection, infusion, implantation, etc.), intraocular administration, and so on.

Such delivery forms and formulations include those for the treatment of a subject as disclosed herein. The pharmaceutical formulations of this invention may further comprise one or more pharmaceutically acceptable excipients. The pharmaceutically acceptable excipients for ocular administration may be ophthalmologically acceptable excipients. In some embodiments, the formulations may provide for sustained delivery of the connexin modulator and/or ocular treatment agent to a selected segment or compartment of the eye. The formulations may, in some embodiments, provide high ocular (including via topical or oral administration, for example) drug bioavailability, be safe and non-toxic, and/or have little systemic side effects or complications at the site of administration. Exemplary polynucleotide formulations for use in the methods of this invention have the ease of localized delivery and ease of administration.

Such delivery forms and formulations include those for the treatment of a subject as disclosed herein. The pharmaceutical formulations of this invention may further comprise one or more pharmaceutically acceptable excipients. The pharmaceutically acceptable excipients for ocular administration may be ophthalmologically acceptable excipients. In some embodiments, the formulations may provide for sustained delivery of the connexin modulator and/or ocular treatment agent to a selected segment or compartment of the eye. The formulations may, in some embodiments, provide high ocular (including via topical or oral administration, for example) drug bioavailability, be safe and non-toxic, and/or have little systemic side effects or complications at the site of administration. Exemplary polynucleotide formulations for use in the methods of this invention have the ease of localized delivery, ease of administration and a “no side effect” profile.

In some embodiments the pharmaceutical formulations of this invention may comprise any of the modulators, e.g. gap junction, hemichannel and/or connexin modulators described herein, for example, a modified or unmodified connexin 43 antisense oligonucleotide or polynucleotide or a modified or unmodified connexin 43 peptide or peptidomimetic. Connexin antisense oligonucleotide that are included in the formulation may be, in some embodiments, an unmodified connexin 43 antisense oligodeoxynucleotide or a modified connexin 43 antisense oligodeoxynucleotide. In some embodiments, the pharmaceutical compositions can include or exclude any of the foregoing.

The modulators, including connexin, connexin hemichannel and connexin gap junction modulators may be present in the formulation in a substantially isolated form. It will be understood that the product may be mixed with carriers or diluents that will not interfere with the intended purpose of the product and still be regarded as substantially isolated. A product of the invention may also be in a substantially purified form, in which case it will generally comprise about 80%, 85%, or 90%, e.g. at least about 88%, at least about 90, 95 or 98%, or at least about 99% of a polynucleotide, for example (or other connexin modulator such as a connexin 43 modulator) or dry mass of the preparation. As described in Example 1, the lufepirsen compositions used in the clinical trial to treat non-healing ocular defects described in Example 2 included 94.2% pure lufepirsen (with an assay of 106%) and had a final drug product purity of 94.9% (with an assay of 97%).

The pharmaceutical formulations for use in the dosing, dose regimens and methods of the invention may comprise one or more pharmaceutically acceptable excipients suitable for delivering the modulators (e.g. lufepirsen), including connexin, connexin hemichannel and connexin gap junction modulators to the eye.

The modulators, including gap junction, hemichannel and/or connexin modulators, of the present invention can also be formulated into microparticle (microspheres, Mps) or nanoparticle (nanospheres, Nps) formulations, or both. In some embodiments of the invention, a nanoparticle or microparticle is used. Such as a particle comprise poly(lactic-co-glycolic acid) (“PLGA”) loaded with the gap junction, connexin, and/or hemichannel modulators, for example, connexin 43 modulators. The modulators can be loaded into the particle volume, onto the particle exterior surface, or both. Particle formulations may be administered, for example, topically to the eye or subconjunctivally. In some embodiments, the particle formulations of any of the gap junction, hemichannel and/or connexin modulators of this disclosure (e.g. connexin 43 modulators) may also comprise liposomes.

A modulator, e.g., a connexin, connexin hemichannel and connexin gap junction modulator, may be administered alone or in combination with one or more additional ingredients and may be formulated into pharmaceutical compositions including one or more pharmaceutically acceptable excipients, diluents and/or carriers. Pharmaceutically acceptable diluents, carriers and/or excipients includes substances useful in preparing a pharmaceutical composition, and are generally safe, non-toxic and neither biologically nor otherwise undesirable. Pharmaceutically acceptable diluents, carriers and/or excipients include those suitable for veterinary use as well as human pharmaceutical use. By way of example, diluents, carriers and/or excipients include solutions, solvents, dispersion media, delay agents, polymeric and lipidic agents, emulsions and the like. By way of further example, suitable liquid carriers, especially for injectable solutions, include water, aqueous saline solution, aqueous dextrose solution, and the like, and vehicles such as liposomes being also especially suitable for administration of agents.

Suitable carriers and diluents include buffered, aqueous solutions, saline, dextrose, glycerol, isotonic saline solutions, for example phosphate-buffered saline, isotonic water, and the like and combinations thereof. In some embodiments, carriers may include propylene glycol, dimethyl isosorbide, and water, and even more particularly, phosphate buffered saline, isotonic water, deionized water, monofunctional alcohols and symmetrical alcohols. In some embodiments pharmaceutically acceptable carrier or diluent may be or contain a thermosetting poloxamer (which may be a liquid or gel, depending on the temperature), a carboxycellulose (e.g. carboxymethylcellulose), a collagen (e.g., a Type I collagen), a collagenous material comprising tropocollagen, a hyaluronan or derived-hyaluronic acid, and/or an oil (e.g., Emu oil). Suitable carriers can be large, slowly metabolized macromolecules such as proteins, polysaccharides, polylactic acids, polyglycolic acids, polymeric amino acids, and amino acid copolymers. The pharmaceutical compositions of this invention do not comprise a connexin modulator in sterile water as the only vehicle. In some embodiments, the formulations will comprise connexin modulators, for example, connexin 43 modulators, for example, 43 antisense oligonucleotide that are included in the formulation may be, in some embodiments, an unmodified or modified connexin 43 antisense oligodeoxynucleotide.

Compositions may take the form of any standard known dosage form including tablets, pills, capsules, semisolids, powders, sustained release formulation, solutions, suspensions, elixirs, aerosols, liquids for injection, gels, creams, transdermal delivery devices (for example, a transdermal patch), inserts such as ocular inserts, or any other appropriate compositions. Persons of ordinary skill in the art to which the invention relates will readily appreciate the most appropriate dosage form having regard to the nature of the condition to be treated and the active agent to be used (e.g., antisense, peptidomimetic, small molecule, etc.) without any undue experimentation.

Preferably the modulator, e.g. connexin modulators of the invention are combined with a pharmaceutically acceptable carrier or diluent to produce a pharmaceutical composition. Connexin modulators may be formulated as compositions for any desired route of administration, including topical, oral, systemic, transdermal, nasal, sublingual, buccal, etc. including formulations for injection (e.g. intracameral injection, subcutaneous injection, intramuscular injection, intravenous injection, etc.).

Pharmaceutically acceptable salts can also be present, e.g., mineral acid salts such as hydrochlorides, hydrobromides, phosphates, sulfates, and the like; and the salts of organic acids such as citrates, acetates, propionates, malonates, benzoates, and the like.

In one aspect the reverse-thermosetting gel may be a liquid at low temperatures, for example at 2-8° C., and which undergoes a reversible liquid to gel transition at temperatures greater than approximately 15° C. Thus, in some embodiments the carrier may be a liquid at temperatures below approximately 15° C., but may form a gel at temperatures above approximately 15° C., such as room temperature or at body temperature. In some instances, the gel is a nonionic polyoxyethylene-polyoxypropylene copolymer gel. In some embodiments the gel is a pluronic gel. The pluronic gel may be, for example, poloxamer 407, also sometimes referred to as Pluronic F-127 (BASF). In some embodiments, the formulations of this invention may comprise from about 15 to about 30% (w/v) gel. In some embodiments, the formulations of this invention may comprise from about 20 to about 25% (w/v) gel. In some embodiments, the formulations of this invention may comprise about 22.6% (w/v) poloxamer 407 gel. In some embodiments, the composition or formation comprises 226.0 mg/mL of a poloxamer (e.g. poloxamer 407). See Example 1.

Other suitable formulations include pluronic gel-based formulations, hydroxymethylcellulose formulations, hydroxyethylcellulose formulations, carboxymethylcellulose (CMC)-based formulations, and hydroxypropylmethylcellulose (HPMC)-based formulations. The composition may be formulated for any desired form of delivery, including topical, instillation, parenteral, intramuscular, subcutaneous, or transdermal administration. Other useful formulations include slow or delayed release preparations.

In addition, if desired substances such as wetting or emulsifying agents, stabilizing or pH buffering agents, or preservatives may also be present. In some embodiments, the pharmaceutical compositions of this invention will comprise suitable ophthalmically acceptable buffers, such as acetate buffers, citrate buffers, phosphate buffers, borate buffers and mixtures thereof. In some embodiments, the buffers useful in the present invention include boric acid, sodium borate, sodium phosphates, including mono, di-and tri-basic phosphates, such as sodium phosphate monobasic monohydrate and sodium phosphate dibasic heptahydrate, and mixtures thereof. In some embodiments, the preservative may be stabilized chlorine dioxide, cationic polymers or quaternary ammonium compounds. In some embodiments the pharmaceutical compositions may also comprise wetting agents, nutrients, viscosity builders, antioxidants, and the like, for example, disodium ethylene diamine tetraacetate, alkali metal hexametaphosphate, citric acid, sodium citrate, sodium metabisulfite, sodium thiosulfate, N-acetylcysteine, butylated hydroxyanisole, butylated hydroxytoluene, polyvinyl alcohol, polyoxamers, polyvinyl pyrrollidone, hydroxypropyl methyl cellulose, hydroxyethylmethyl cellulose, and mixtures thereof and mixtures thereof. In some embodiments, the pharmaceutical formulations of this invention will not include a preservative. In some embodiments, the connexin modulator composition or formulation comprises sodium phosphate dibasic heptahydrate or potassium phosphate, monobasic or both. See Example 1.

Where the modulator, e.g. gap junction, connexin, and/or hemichannel modulator is a nucleic acid, such as a polynucleotide, uptake of nucleic acids by mammalian cells may be enhanced by the of known transfection techniques including the use of transfection agents. Such techniques may be used with certain anti-connexin agents, including polynucleotides. The formulation that is administered may contain such transfection agents. Examples of useful transfection agents include cationic agents (for example calcium phosphate and DEAE-dextran) and lipofectants (for example Lipofectam™ and Transfectam™), and surfactants.

The connexin, gap junction, and/or hemichannel modulators, for example, connexin 43 modulators may also be formulated to provide controlled release to the eye. In some embodiments of this invention, the formulations may be immediate, or extended or sustained release dosage forms, e.g., for release within several hours, within one day or, for example, within 1-2 days.

The compositions may be formulated in accordance with standard techniques known in the art, including those as may be found in such standard references as Gennaro A R: Remington: The Science and Practice of Pharmacy, 20th ed., Lippincott, Williams & Wilkins, 2000, for example.

Any container suitable for storing and/or administering a pharmaceutical composition may be used in a combination product of the invention. Suitable containers will be appreciated by persons skilled in the art. By way of example, such containers include vials and syringes. The containers may be suitably sterilized and hermetically sealed.

In some embodiments, administering a connexin modulator (for example, a connexin 43 modulator or connexin 45 modulator, preferably a connexin 43 modulator, e.g. lufepirsen), to the eye of a subject provides a therapeutically effective amount of the connexin modulator to the eye or specific compartment or portion of the eye by means of administration as desired and appropriate depending on the nature of the connexin modulator, including but not limited to topical administration.

Therapeutically effective amounts include but are not limited to the doses described herein. Described doses and other therapeutically effective amounts are administered in one or more of the therapeutically effective dose regimens described herein.

### Administration

Administration of a modulator, e.g. a connexin modulator, a gap junction channel modulator and/or a hemichannel modulator compounds and compositions may be administered by one of the following routes: oral, topical, systemic (including intravenous, intra-arterial, intra-peritoneal, transdermal, intranasal, or by suppository), parenteral (including intramuscular, subcutaneous, or intravenous or intra-arterial injection), and the like. In some embodiments, the connexin modulator, gap junction channel modulator and/or hemichannel modulator compounds and compositions are administered topically. In some embodiments, the connexin modulator, gap junction channel modulator and/or hemichannel modulator compounds and compositions are administered systemically. In some embodiments, the connexin modulator, gap junction channel modulator and/or hemichannel modulator compounds and compositions are administered orally. In some embodiments, the connexin modulator, gap junction channel modulator and/or hemichannel modulator compounds and compositions are administered by intracameral injection formulation and route of administration, as this may be a preferred route of administration (or if topical is less effective) for treatment, e.g. of uveitis and Fuchs' dystrophy.

In some embodiments of the methods of this invention, the connexin modulator, connexin gap junction modulator and/or connexin hemichannel modulator, may be administered locally by topical administration to the eye of the subject. In some embodiments, the connexin modulator is administered by topical, corneal and/or subconjunctival administration, or by local injection. Topical formulations of the gap junction, hemichannel and/or connexin modulators can comprise ointments, gels, which may be, for example, thermosetting gels, drops, sprays, liquids and powders, or a sustained or non-sustained release dosage form. Bioavailable systemically administered connexin modulators, connexin gap junction modulators and/or connexin hemichannel modulators may be administered, with or without concomitant local administration to the eye, for example, by oral administration, intravenous administration, enterally through the gastrointestinal system (e.g. oral, rectal, sublingual, buccal), parenterally (e.g. intravenously, intramuscularly, subcutaneously, transdermally), intranasally, by nasal inhalation, and by mouth inhalation. In some embodiments, non-topical administration, including intraperitoneal administration or orally or by parenteral administration, may be employed provided that a therapeutically effective dose contacts the eye or portion thereof (e.g. cornea) to be healed. In some embodiments, in addition to administration of a connexin modulator by topical administration, orally available connexin modulators (e.g., tonabersat) administration may also be administered by intraperitoneal, oral or parenteral administration, provided that a therapeutically effective dose contacts the eye.

In some embodiments, administering a connexin modulator, for example, a connexin expression modulator, a peptidomimetic or small molecule gap junction modulator and/or a hemichannel modulator (for example, a connexin 43 modulator or a modulator or any other connexin in the eye, the cornea, the corneal epithelium or in blood vessels), to the eye of a subject provides a therapeutically effective amount of the connexin modulator(s) to the eye. In some embodiments, a bandage contact lens is applied to the eye following administration of s connexin modulator(s), whether topically or by another route of administration. In some embodiments, the connexin modulator is placed in a bandage contact lens which is the applied to the eye of the subject with a non-healing ocular defect or disorder (e.g. a PED or PCED) or other disease, disorder or condition described or referenced herein. In some embodiments, the connexin modulator is administered under an amniotic membrane. In some embodiments, an amniotic membrane is applied to the eye following administration of the connexin modulator. The modulator may be a connexin 43 modulator. In some embodiments, the connexin modulator is embedded in a matrix comprising a bandage contact lens or other implantable device. In some embodiments, the connexin modulator matrix (e.g. bandage contact lens connexin modulator matrix) provides for slow or sustained release of the connexin modulator contact lens. As used herein, “matrix” includes for example, matrices such as polymeric matrices, biodegradable or non-biodegradable matrices, and other carriers useful for making implants or applied structures for delivering the non-healing ocular and/or or corneal surface disorder or neuropathic treatment connexin modulators. Compositions and methods for the preparation of drug-containing contact lenses have been developed and are known in the art. See e.g. Wang Z, et al., Novel Contact Lenses Embedded with Drug-Loaded Zwitterionic Nanogels for Extended Ophthalmic Drug Delivery. Nanomaterials 11:2328 (2021).

In some embodiments of the present invention, the subject with a non-healing surface defect or disorder, e.g., a PCED, has an amniotic membrane graft. In some embodiments, the gap junction modulator or the hemichannel modulator, e.g. lufepirsen, is administered under an amniotic membrane graft. In some embodiments, an amniotic membrane graft is applied to the non-healing surface disorder, e.g. a PCED, after application of the gap junction modulator or the hemichannel modulator, e.g. lufepirsen.

### Articles of Manufacture/Kits

In another embodiment of the invention, an article of manufacture, or “kit”, containing materials useful for treating a non-healing ocular surface or corneal defect and disorder (e.g. a PED or PCED) is provided. In another embodiment of the invention, an article of manufacture, or “kit”, is provide that containins materials useful for treating uveitis, blepharitis, Sjögren's syndrome-associated keratoconjunctivitis sicca, Fuchs' dystrophy, and/or dry eye disease (e.g., evaporative dry eye, aqueous-deficient dry eye, and decreased wettability dry eye).

The kit comprises a container with a composition comprising one or more modulators, e.g. a connexin modulator such as a connexin antisense, a connexin peptidomimetic and/or a small molecule hemichannel blocker, for example. The kit may further comprise a label or package insert, on or associated with the container, which includes instructions for dosing as described herein. The term “package insert” refers to instructions customarily included in commercial packages of therapeutic products that contain information about the indications, usage, dosage, administration, contraindications and/or warnings concerning the use of the modulator, which may be provided physically or via an online link, for example. Suitable containers include, e.g., bottles, vials, etc. The container may be formed from a variety of materials such as glass or plastic. In some embodiments, the label or package insert indicates that the composition is used for treating a non-healing ocular surface or corneal surface defect and disorder (e.g. a PED or PCED). In some embodiments, the label or package insert indicates that the composition is used for treating uveitis, blepharitis, Sjögren's syndrome-associated keratoconjunctivitis sicca, Fuchs' dystrophy, and all forms of dry eye disease (e.g., evaporative dry eye, aqueous-deficient dry eye, and decreased wettability dry eye).

A kit may comprise one or more pharmaceutical compositions, in separate vessels, or a partitioned vessel, together with packaging and instructions for use. The kit may also comprise a pharmaceutically acceptable carrier. In some embodiments the kit may also include components for administering the pharmaceutical compositions, for example, a syringe, needle, microneedle, etc. In some embodiments, the kit comprises a bandage contact lens for application to the subject following administration of a connexin modulator or for using in applying the connexon modulator. The connexin modulator will be dosed on separate days and may include packaging and/or instructions for chronologically staggered administration in accordance with methods of the invention.

### Manufacture

The polynucleotides of this invention can be manufactured using solid-phase chemistries for synthesizing oligonucleotides, chemistries known in the art for synthesizing and preparing peptides and peptidomimetics, and chemistries know in the art for synthesizing organic compounds (e.g. tonabersat, and other compounds of Formula I and Formula II). In one aspect, the formulations of this invention will comprise a salt of the polynucleotides of this invention, such as the sodium salt of the polynucleotides of this invention. The kit may also comprise a pharmaceutically acceptable carrier. In one embodiment the formulation may comprise the sodium salt of a polynucleotide having any one of SEQ.ID.NO: 1-16 or a portion of SEQ ID NO: 17, for example. In some embodiments, the polynucleotide having any one of SEQ.ID.NO: 1-16 may be a modified oligodeoxynucleotide having any one of SEQ.ID.NO: 1-16, or a portion of SEQ ID NO:17.

In some embodiments, the formulations of this invention are substantially pure. By substantially pure is meant that the formulations comprise less than about 10%, 5%, or 1%, and preferably less than about 0.1%, of any nucleotide or non-nucleotide impurity. In some embodiments the total impurities, including metabolites of the connexin 43 modulating agent, will be not more than 15%. In some embodiments the total impurities, including metabolites of the connexin 43 modulating agent, will be not more than 12%. In some embodiments the total impurities, including metabolites of the connexin 43 modulating agent, will be not more than 11%. In other embodiments the total impurities, including metabolites of the connexin 43 modulating agent, will be not more than 10%. See e.g. Example 1, which describes the preparation of lufepirsen compositions with a final drug product purity of 94.9%.

Sterile compositions comprising the connexin 43 modulating agents of this invention prepared using aseptic processing by dissolving the anti-connexin modulating agent in the formulation vehicle. In one embodiment, the formulation may also be sterilized by filtration. Excipients used in the manufacture of the formulations of this invention are widely used in pharmaceutical products and released to pharmacopeial standards.

### EXAMPLES

The below work was undertaken to evaluate whether connexin modulation could be used in patients for the treatment of non-healing ocular defects and disorders, including ocular persistent epithelial defects and persistent corneal epithelial defects.

This work described in the Examples was carried out over several years in multiple countries in an effort to work to discover and identify previously unknown but clinically useful therapeutic doses and dosing regimens in a randomized, double-blind placebo-controlled human clinical trial.

The successful results of this work and these efforts are described.

### Example 1

### Preparation of Lufepirsen® (Lufepirsen) Compositions for Topical Application in Subjects with Persistent Corneal Epithelial Defects

Compositions comprising the connexin 43 modulator, lufepirsen (5′-GTA ATT GCG GCA AGA AGA ATT GTT TCT GTC-3′ [SEQ ID NO: 1]) for topical ocular administration were prepared using by dissolving the anti-connexin modulating agent in the formulation vehicle and buffers added, as shown in the table below.

| | Reference to | | | |
|---|---|---|---|---|
| Ingredient | Standards | Concentration | | Function |
| Lufepirsen | API-SPC- | 0.6 or 0.06 | | Active |
| | C06A | mg/mL | | |
| Poloxamer 407 | USP NF | 226.0 | mg/mL | Thermosetting |
| | | | | excipient |
| Sodium phosphate | USP NF | 0.99 | mg/mL | Buffer |
| dibasic heptahydrate | | | | |
| Potassium phosphate, | USP NF | 0.25 | mg/mL | Buffer |
| Monobasic | | | | |

| | | | |
|---|---|---|---|
| Water for Injection | USP | QS (dose | Diluent |
| | | dependent) | |

USP = United States Pharmacopeia; NF = National Formulary

Compositions comprising 0.6 mg/ml or 0.06% of the lufepirsen connexin 43 modulator were prepared to a final volume of 300 μL for administration using by dissolving 0.18 mg lufepirsen or 19.40491591203 micromolar (μM) [005 API (lot C06A14001)] in the poloxamer pharmaceutical formulation vehicle with buffers.

Compositions comprising 0.06 mg/ml or 0.006% of the lufepirsen connexin 43 modulator were prepared to a final volume of 300 μL for administration using by dissolving 0.018 mg lufepirsen or 1.940491591203 micromolar (μM) [005 API (lot C06A14001)] in the in the poloxamer pharmaceutical formulation vehicle with buffers.

Lufepirsen purities were 94.2% with an assay of 106% and drug product purities were 94.9% with an assay of 100%. The lufepirsen compositions were used in the clinical trial as described in Example 2.

### Example 2

### A Phase 2, Randomized, Prospective, Double-Masked, Vehicle-Controlled Study to Assess the Efficacy and Safety of Lufepirsen® (Lufepirsen) Applied Topically in Subjects with Persistent Epithelial Defects (Ped) Resulting from Ocular Chemical and/or Thermal Injuries

This Example describes the use of an exemplary connexin modulator (lufepirsen) in a randomized, prospective, vehicle-controlled, double-masked, human clinical trial to treat non-healing ocular surface defects. Lufepirsen is a connexin 43 antisense oligonucleotide according to SEQ ID NO:1. The vehicle comparator in the trial was Pluronic F-127.

Study Subjects: A total of 35 subjects were enrolled in this clinical trial. All eligible subjects presented with a non-infected, persistent epithelial defect (PED) of the cornea as a result of a chemical and/or thermal ocular injury, that was refractory to current standard of care for at least 14 days. Of the 35 subjects, 24 subjects received topical ocular dose administrations of LUFEPIRSEN and 11 subjects received Vehicle. There were 12 subjects who did not re-epithelialize, or did not remain re-epithelized for 28 days that received either one (Salvage Day 1) or two administrations (Salvage Day 8) of LUFEPIRSEN 0.06% in an open-label fashion. The results are described below.

Study Objective: The objective of this clinical protocol was to assess the efficacy and safety of two topical ocular dose concentrations of LUFEPIRSEN in a novel protocol for treatment of non-healing corneal PEDs resulting from severe chemical and/or thermal ocular injuries.

Study Endpoints: The primary, secondary and other endpoints evaluated in this human clinical are set forth below:

### Primary Endpoint:

Corneal epithelial recovery, defined as a cornea that re-epithelializes by Day 28 of treatment and remains re-epithelialized for at least 28 days after initial re-epithelialization was first recorded, as assessed by the Investigator.

### Secondary Endpoints:

The time to epithelialization defined as the time from randomization to the time of initial re-epithelialization.
Improvement of visual acuity from baseline over the course of the study period.
Number of LUFEPIRSEN treatment doses required for recovery of the corneal epithelium.

### Exploratory Endpoints:

Change from baseline in PED area defined as the largest diameter times the largest one perpendicular to it, within the confines of the epithelial defect.
Corneal re-epithelialization, defined as a cornea that has re-epithelialized by Day of treatment.
Change from baseline in ocular symptoms.
Primary, secondary, and exploratory endpoints were also assessed (as applicable) for the open-label section of the study.
Descriptive comparison of intrasubject corneal re-epithelization to fellow eye in subjects with bilateral injuries

### Safety:

Incidence of Treatment Emergent Adverse Events.

Treatment Dosing: The study comprised of a 28-day Treatment Period, followed by a Post--treatment Follow-up Period of at least 28 days to evaluate both safety and re-epithelization status (durability), both of which were double-masked (i.e., double-blinded). Once determined to be eligible, subjects were randomized to receive either LUFEPIRSEN 0.06% or LUFEPIRSEN 0.006% or Vehicle in a 1:1:1 ratio respectively. Investigational product was applied under a bandage contact lens (BCL) and to the fornices of the study eye at the following time points during the Treatment Period:
Day 1
Day 2
Day 14 (if the cornea has not re-epithelialized)

Salvage Dosing: However, if by Day 28 of the Treatment Period a subject's cornea had not fully re-epithelialized, irrespective of the investigational product assignment at randomization, that subject was entered into the open-label period of the study and received up to two salvage dose applications of LUFEPIRSEN 0.06% applied under a BCL and to both fornices of the eye, with LUFEPIRSEN 0.06% applied on Salvage Day 1 and, if re-epithelialization was still not achieved, on Salvage Day 8 of the open-label section of the study. Twelve subjects receiving salvage therapy were followed up to 14 days from first salvage dose application to determine if re-epithelialization occurred (Day 41) and then returned >28 days, to confirm that re-epithelialization was maintained (durability).

For each subject, the 28-day masked Treatment Period ended when EITHER, corneal re-epithelialization was first recorded following up to a maximum of three masked applications of investigational product OR subjects enters the open-label section of the study as a result of an unhealed PED at Day 28.

Those subjects who achieved re-epithelialization immediately began the 28-day Post-Healing Follow-up Period (with no further investigational product applied). The aim of the post epithelialization follow-up period was to assess the durability of the epithelium.

For those subjects that achieve re-epithelialization but epithelialization was NOT sustained during the 28-day Post-treatment Follow-up Period, the subject was eligible to receive the salvage dose(s) of LUFEPIRSEN 0.06% and enter the open-label section of the study (Salvage Day 1). If re-epithelialization was not achieved by Salvage Day 8 of the open-label section of the study, a second dose of LUFEPIRSEN 0.06% was applied. If re-epithelialization occurs within 14-days of the first salvage dose, a final assessment of the epithelium durability was performed 28 days later.

Subjects also exited the study if epithelialization was not achieved within 14 days of receiving salvage dose applications of LUFEPIRSEN 0.06% in the open-label section of the study. There were 12 subjects who received Salvage (high dose open label LUFEPIRSEN) in the NEX-PED-005 study. Eleven subjects received 1 dose at Day 28, with one subject receiving doses (Day 28 and Day 35).

The maximum duration for study participation for a subject was 98 days (+2 days). this assumes re-epithelialization on day 28 of the masked treatment period, enabling the post-healing follow-up period to start. in cases where durability was not sustained 28 days later, the subject then became eligible for the 0.06% LUFEPIRSEN salvage dose(s), enabling that subject to potentially participate in the open-label section of the study for up to a further 41 days (14 days+28 days (+2 days)). Table 1 shows the schedule of assessments for details of the visit schedule and the procedures to be conducted at each study visit.

Investigational Product Administration for Randomized Subjects: Each investigational product dose application comprised a volume to fill the BCL (100 μL) and cover the inner surfaces of the superior and inferior fornices (100 μL instilled per fornix). The eye was then covered with a double eye pad and taped shut for approximately 8 hours.

No topical ocular standard of care treatment was permitted 2 hours before or 8 hours following investigational product administration. Outside of this period, subjects continued to receive the standard of care regimen prescribed to them by the Investigator.

Three treatment arms were evaluated in the study. Randomization was in a 1:1:1 ratio:
GROUP A—LUFEPIRSEN 0.06%
GROUP B—LUFEPIRSEN 0.006%
GROUP C—Vehicle

There was a single application of investigational product on Day 1 and Day 2. A potential additional application of investigational product was scheduled at Day 14 if re-epithelialization has not occurred.

If re-epithelialization of the study eye had not occurred at the end of the 28-day masked Treatment Period, or following initial re-epithelialization durability was not maintained, subjects received up to two salvage dose applications of LUFEPIRSEN 0.06% in the open-label section of the study.

Study Description: Potentially eligible subjects presented with a clinically non-infected PED as a result of a severe chemical and/or thermal injury refractory to standard of care. Clinical signs of the ocular injury include de-epithelialization, conjunctival inflammation (hyperemia), limbal ischemia and corneal edema.

Before the entry of a subject was considered, the PED must have been present for at least 14 days, with the day of injury being counted as the first day, and in the opinion of the Investigator, the defect must not have shown a clinically meaningful improvement in healing despite conventional standard of care over a period of at least 14 days. Conventional standard of care included: antibiotics, steroid drops, cycloplegic agents, ascorbic acid, amniotic membrane placement, debridement of the necrotic epithelium, etc.

Subject consent to participate in the study was obtained by the Principal Investigator or suitably qualified designee before any protocol-specific procedures were undertaken. Eligibility assessments and baseline evaluations were performed prior to Day 1 of the Treatment Period, with final eligibility checks such as confirmation of a negative urine pregnancy test for women of childbearing potential and pre-treatment measures of the PED dimensions performed before subjects are randomized to treatment. The eligibility assessment and Day 1 visits may be combined at the Investigator's discretion, when subject's documented medical history confirmed the epithelial defect had been present for over 14 days and was non-responsive to SOC. The visits may also be combined assuming the medical history has been confirmed and the subject was experiencing logistical challenges such as, subject needs to travel long distances to the site or due to transport difficulties and restrictions as a result of the COVID-pandemic, PROVIDING all assessments required prior to randomization have been undertaken, documented, and subject eligibility was confirmed.

The first investigational product application occurred following randomization on Day 1 of the Treatment Period, with the second application administered on Day 2. For each single application, a BCL filled with 100 μL investigational product was applied immediately on the study eye followed by an application of 100 μL into each of the superior and inferior fornices. The eye was then covered with a double eye pad and taped shut for approximately 8 hours. Importantly, subjects continued to receive SOC prescribed to them over the duration of the treatment period EXCEPT 2 hours before IP application and 8 hours after.

Each subject then attended twice-weekly assessment visits until corneal re-epithelialization occurs.

If re-epithelialization of the defect had NOT occurred by Day 14, another single application of masked investigational product was administered.

If corneal re-epithelialization has NOT occurred by Day 28 of the masked Treatment Period, subjects received up to two salvage dose applications of LUFEPIRSEN 0.06% in the open-label section of the study. If re-epithelialization has not occurred within 14 days of the first salvage dose application(s) of LUFEPIRSEN 0.06%, the subject exited the open-label section of the study.

Once re-epithelialization has occurred, regardless of time frame within the masked Treatment Period, the subject entered the Post-healing Follow-up Period and continue to wear the BCL for 2 weeks to allow the new epithelium to become anchored to the basement membrane and avoid premature traumatic detachment of the cell layer. Subjects were assessed again 28 days following initial re-epithelialization to confirm durability of the epithelium. The subject then exited the study having completed all visits.

If, after initial epithelialization, the epithelium breaks down within the 28-day Post-treatment Follow-up period, the subject received up to two salvage dose applications of LUFEPIRSEN 0.06%, given 7 days apart, in the open-label section of the study.

If a re-epithelialized cornea develops within 14 days of the first open-label salvage dose application, these subjects will also be followed for 28 days to assess durability of re-epithelization but was analyzed outside of the masked study population. See FIG. 1. At each study visit following randomization until the end of the Treatment Period, the subject will undergo assessments as detailed in a Schedule of Assessments. Assessments included slit lamp examination to assess the condition of the anterior segment of the eye (including the eyelids, sclera, conjunctiva, cornea, anterior chamber, and lens), photography, measurement of the epithelial defect, visual acuity, intraocular pressure and the ocular symptom questionnaire. The occurrence of any Treatment Emergent Adverse Events will also be assessed at each study visit, and up to 30 days following the final application of investigational product.

Standard of Care during the Treatment Period and the Open-label section of the study: From the start of Day 1 through to the END of the Treatment Period EXCEPT for 2 hours before and the 8-hour period after investigational product administration, the subjects continued to receive the standard of care regimen prescribed to them by the Investigator at the study site. Details of the medications administered, doses, and dosing regimens were recorded in the source documentation and case report form.

Bandage contact lenses (BCL) for IP administration was provided by the Sponsor and were to be worn during the Treatment Period, replaced only when a new BCL was used for IP administration on dosing days, or if indicated, e.g., discomfort or BCL falling out.

Systemic standard of care was not defined by the protocol but by the Investigator's or study site routine practice. However, systemic medications and regimen for the treatment of the PED were to remain unchanged for the duration of the study, until re-epithelialization or study exit.

Use of topical anesthetic drops was only permitted for ophthalmic assessments conducted at each study visit.

Inclusion Criteria: A subject was eligible for inclusion in the study only if ALL of the following criteria applied.
1. Male and female of any age.
2. Clinically non-infected, corneal persistent epithelial defect (PED) as a result of a severe chemical and/or thermal ocular injury in one or both eyes.
3. Documentation of the PED being non-responsive to current standard of care for at least 14 days with day 1 being the day of injury.
4. An epithelial defect measuring at least 2 mm along the largest diameter at Day 1 of the Treatment Period.
5. Providing written informed consent and ability to comply with the visit and dosing schedule.

The blind was broken, and an analysis was conducted when 35 randomized subjects (LUFEPIRSEN 0.06% (n=12), LUFEPIRSEN 0.006% (n=12)) had completed study. Utilizing the full data set, 66.7% (8 of 12) of LUFEPIRSEN 0.06% subjects and 66.7% (8 of 12) of LUFEPIRSEN 0.006% subjects achieved corneal epithelial recovery. In contrast, Vehicle subjects achieved only 27.3% (3 of 11) recovery, and each of these 3 subjects who recovered did so after receiving only 2 doses of Vehicle on Days 1 and 2, while 3 subjects who did not recover also received 2 doses with the remaining non-recovering Vehicle cohort subjects receiving 3 doses of Vehicle.

Thus, subjects with a non-healing ocular surface defect receiving the connexin modulator LUFEPIRSEN (n=24) were almost two and half times (2.44 times) more likely to heal on application of a connexin modulator and exhibited a 39.4% comparative improvement in corneal epithelial recovery over the VEH group (n=11). These findings are clinically meaningful and, despite the small sample population, clearly approached statistical significance (Fisher's exact test: p=0.065). In general, treatment was well tolerated, with TEAEs across all 3 treatment groups being similarly characterized as mild to moderate in severity and unlikely/unrelated to study drug. A SAE deemed not related to study drug was reported.

These data from a randomized, prospective, vehicle-controlled, double-masked, human clinical trial to treat non-healing ocular surface defects corroborate the use of a connexin modulator (e.g., LUFEPIRSEN) in the dose regimens described herein using the doses described herein, or in a dose regimen as described herein using a different, therapeutically effective dose, to durably heal a non-healing or persistent ocular surface defect, including persistent corneal epithelial defects. The data establish both safety and clinical proof of concept for the unique connexin modulators doses and dose regimens described and claimed herein, and confirm their utility in the therapeutic management of patients with a non-healing ocular surface or corneal defect or disorder.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents. Reference to any applications, patents and publications in this specification is not, and should not be taken as, an acknowledgment or any form of suggestion that they constitute valid prior art or form part of the common general knowledge in any country in the world.

The specific methods and compositions described herein are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of” may be replaced with either of the other two terms in the specification. Also, the terms “comprising”, “including”, containing “, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms” a,” “an,” and “the” include plural reference unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants. Furthermore, titles, headings, or the like are provided to enhance the reader's comprehension of this document and should not be read as limiting the scope of the present invention. Any examples of aspects, embodiments or components of the invention referred to herein are to be considered non-limiting.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or embodiments of the invention are described in terms of Markush groups, those skilled in the art will recognize that the invention is also thereby described in terms of any individual member or subgroup of members of the Markush group.

1. A method for treating a subject for a non-healing ocular surface defect, comprising administering to an eye of the subject a composition comprising a connexin 43 antisense oligonucleotide selected from the group consisting of SEQ ID NOs: 1 to 16, wherein the composition is administered at a dose of the connexin 43 antisense oligonucleotide of about 0.06 mg/mL or about 0.6 mg/mL at least once on day 1 and at least once on day 2.

2. The method of claim 1, wherein the non-healing ocular surface defect is a persistent epithelial defect (PED).

3. The method of claim 1, wherein the non-healing ocular surface defect is a persistent corneal epithelial defect (PCED).

4. The method of claim 1, wherein the non-healing ocular surface defect results from a chemical injury, a thermal injury, or inflammation.

5. The method of claim 1, wherein the connexin 43 antisense oligonucleotide is administered by topical administration.

6. The method of claim 1, further comprising placing a bandage contact lens over the non-healing ocular surface defect.

7. The method of claim 1, wherein the connexin 43 antisense oligonucleotide is formulated with a pluronic gel.

8. The method of claim 7, wherein the composition comprises from about 15% (w/v) to about 30% (w/v) of the pluronic gel.

9. The method of claim 8, wherein the composition comprises from about 20% (w/v) to about 25% (w/v) of the pluronic gel.

10. The method of claim 9, wherein the composition comprises from about 22.6% (w/v) of the pluronic gel.

11. The method of claim 1, wherein the composition comprises a phosphate buffer.

12. The method of claim 1, wherein the composition comprises sodium phosphate dibasic heptahydrate and potassium phosphate, monobasic.

13. The method of claim 1, wherein the composition comprises about 22.6% (w/v) poloxamer 407, about 0.99 mg/mL sodium phosphate dibasic heptahydrate, and about 0.25 mg/mL potassium phosphate, monobasic.

14. The method of claim 1, further comprising administering the dose of the connexin 43 antisense oligonucleotide to the eye of the subject at least once on about day 7.

15. The method of claim 14, further comprising administering the dose of the connexin antisense oligonucleotide to the eye of the subject at least once on about day 14.

16. The method of claim 15, further comprising administering the dose of the connexin antisense oligonucleotide to the eye of the subject at least once on about day 21.

17. The method of claim 16, further comprising administering the dose of the connexin antisense oligonucleotide to the eye of the subject at least once on about day 28.

18. The method of claim 17, further comprising administering the dose of the connexin antisense oligonucleotide to the eye of the subject at least once on about day 35.

19. The method of claim 1, wherein the connexin 43 antisense oligonucleotide is lufepirsen (SEQ ID NO: 1).

20. The method of claim 19, wherein the composition comprises about 0.06 mg/mL lufepirsen, about 22.6% (w/v) poloxamer 407, sodium phosphate dibasic heptahydrate, and potassium phosphate, monobasic.

21. The method of claim 19, wherein the composition comprises about 0.6 mg/mL lufepirsen, about 22.6% (w/v) poloxamer 407, sodium phosphate dibasic heptahydrate, and potassium phosphate, monobasic.

22. The method of claim 19, further comprising administering the dose of lufepirsen to the eye of the subject at least once on about day 7, at least once on about day 14, and at least once on about day 21.

23. The method of claim 22, further comprising administering the dose of lufepirsen to the eye of the subject at least once on about day 28.

24. The method of claim 23, further comprising administering the dose of lufepirsen to the eye of the subject at least once on about day 35.

25. The method of claim 22, further comprising administering one or more salvage doses of lufepirsen to the eye of the subject.
